(19) United States
(12) Patent Application Publication
(10) Pub. No.: US 2019/0214145 A1
(43) Pub. Date: Jul. 11, 2019

(54) Method and systems for creating and screening patient metabolite profile to diagnose current medical condition, diagnose current treatment state and recommend new treatment regimen

(71) Applicants: Itzhak Kurek, San Francisco, CA (US); Robert McKee, Austin, TX (US); Mike Siani-Rose, San Francisco, CA (US)

(72) Inventors: Itzhak Kurek, San Francisco, CA (US); Robert McKee, Austin, TX (US); Mike Siani-Rose, San Francisco, CA (US)

(21) Appl. No.: 16/245,249

(22) Filed: Jan. 10, 2019

Related U.S. Application Data
(60) Provisional application No. 62/615,443, filed on Jan. 10, 2018.

(51) Int. Cl. G16H 50/30; G16H 50/70; G16H 50/50; G16H 50/20; G16B 50/30; G16B 50/20; G16B 40/20; A61K 31/366; A61P 29/02; G01N 33/50

(52) U.S. Cl. CPC G16H 50/30; G01N 2800/70; G16H 50/50; G16H 50/20; G16B 50/30; G16B 50/20; G16B 40/20; A61K 31/366; A61P 29/02; G01N 33/50; G01N 2800/52; G01N 2800/50; G01N 2800/56; G01N 2800/54; G16H 50/70

(57) ABSTRACT

Disclosed are methods and systems for building a database of metabolite profiles correlated with disease states and treatment regiments, then defining an individual patient's metabolite profile, and then screening the patient's profile against the database to recommend potential effective treatment regimens.

### CROSS-REFERENCE TO RELATED APPLICATIONS

This application claims priority to and the benefit of the filing of U.S. Provisional Patent Application Ser. No. 62/615,443 entitled “Method and systems for creating and screening patient metabolite profile to diagnose current medical condition, diagnose current treatment state and recommend new treatment regimen”, by Itzhak Kurek, Michael Siani-Rose, and Robert McKee, filed on Jan. 10, 2018, and the specification, figures, and claims thereof are incorporated herein by reference.

### BACKGROUND

Medical cannabis refers to using the whole, unprocessed cannabis plant or its basic extracts to treat medical conditions. The increasing usage of cannabis from different cultivated varieties, the lack of standards for plant contents and dosage, and the high number of active components significantly complicate the conventional medical approach: identification of a single active ingredient for effective treatment.

Metabolites are the end products of cellular processes and their levels can be regarded as the ultimate measurable response of biological systems to physiological changes. Thus metabolomics-based biomarkers reflect aspects of the physiological state and provide diagnostic tools for clinical routines (Zhang et al 2012).

The discovery process of metabolomics-based biomarkers includes a comprehensive analysis in which all the metabolites of a biological system (such as sugars, amino acids, lipids, steroids and triglycerides) are traced and quantified. The two major approaches for discovery are: 1) targeted metabolomics—the quantitative measurement of specifically defined chemically characterized and biochemically annotated molecules involved in known metabolic pathways; and 2) un-targeted metabolomics: quantitative measurement of the dynamic multi-parametric metabolic response of biological systems to physiological changes (Fien, 2002; Commisso et. al., 2013).

In order to avoid exclusion of any metabolite, the metabolomics process requires: 1) A well-conceived experimental design; 2) Sample preparation procedures; and 3) Analytical techniques.

Predictive biomarkers or biomarkers indicative of efficacy are defined for a specific treatment, such as medical cannabis for a well-characterized condition. The biomarker values determined using sensitive and reliable quantitative procedures are associated with the differential efficacy or adverse effect of that treatment.

The process of extraction and analysis of complex metabolomics profiling data performed by machine learning methods generates valuable information on the pattern and function of biomarkers that are often associated with the same or similar responses to biological changes at the population, sub-population and individual levels. This is similar to the stratified medicine approach of identifying subgroups of patients with distinct mechanisms of disease or particular responses to treatments (Malottki et. Al., 2014).

There is a need for a powerful new bioinformatics platform to make use of machine learning methods to combine untargeted and targeted metabolomics, cultivar genetic markers and user characteristics databases to identify user biomarkers derived from the consumption of cannabis. The platform may be used to recommend cultivar type and dosage based on individual factors such as medical conditions, age, gender, ethnicity and body mass index (BMI).

### SUMMARY

The method and system comprise a set of data bases or tables which capture the relationships between (1) disease states or medical indications and metabolites and treatments; (2) human patient metabolite tables which comprise metabolites detected pre-treatment, immediately post-treatment, then for different timestamps post-treatment; (3) cultivar or pharmaceutical source tables linked to known metabolites produced by the cultivar either in the plant or in the context of the treated patient. These individual databases or tables comprise the knowledge base.

The system is then built using an artificial intelligence, machine learning, neural network, or fuzzy logic algorithm, or other method, to build a model of the data in the database.

The method comprises the identification of metabolites in a particular patient, then screening them against the model using a classifier system. The system then produces matches against known entries in the knowledge base. These matches will provide a recommended course of action, such as a recommended cultivar type and dosage, which is based on previous patient data represented in the model, which is based on hard evidence in the databases. Alternatively, these matches will provide a diagnosis of a condition (e.g., inflammatory response caused to infection or auto-immune disease).

### BRIEF DESCRIPTION OF DRAWINGS

The accompanying drawings, which are incorporated herein and constitute part of this specification, illustrate exemplary embodiments of the claims, and together with the general description given above and the detailed description given below, serve to explain the features of the claims.

FIG. 1. illustrates a method or process for (1) screening a patient sample against a model (the model trained on a knowledge database that combines medical indications, metabolite tables, and cultivar source information) and (2) producing a recommended treatment or (3) producing a diagnosis.

FIG. 2a. illustrates features of a table including a plurality of records having a plurality of fields consistent with implementations of the current subject matter.

FIG. 2b. illustrates features of a table including a plurality of records having a plurality of fields consistent with implementations of the current subject matter.

FIG. 2c. illustrates features of a table including a plurality of records having a plurality of fields consistent with implementations of the current subject matter.

FIG. 2d. illustrates features of a table including a plurality of records having a plurality of fields consistent with implementations of the current subject matter.

FIG. 2e. illustrates features of a table including a plurality of records having a plurality of fields consistent with implementations of the current subject matter.

FIG. 2f. illustrates features of a table including a plurality of records having a plurality of fields consistent with implementations of the current subject matter.

FIG. 2g. illustrates features of a table including a plurality of records having a plurality of fields consistent with implementations of the current subject matter.

FIG. 3. illustrates a processing overview of the cultivar recommendation engine.

FIG. 4. Illustrates a processing overview of dosage recommendation engine.

FIG. 5 illustrates a heat map of biomarkers of efficacy values.

FIG. 6 illustrates a flow chart representing one method of an embodiment of the current subject matter.

FIG. 7. Illustrates a database schema of one embodiment of the current subject matter.

FIG. 8. illustrates a Venn Diagram of the number of biomarker candidates that significantly change 10 min post-consumption of cannabis.

FIG. 9. illustrates the levels of the biomarkers.

FIG. 10. illustrates the TCA metabolic pathway.

FIG. 11. illustrates a system environment diagram, in which various embodiments may be implemented.

### DETAILED DESCRIPTION

Biomarker: Metabolite that exhibits statistically significant values difference between iMPpr (pre-consumption) to one or more time points.

Ratio: Ratio of 2 or more metabolites that exhibits statistically significant differences between 2 or more time points.

Cultivar and intoxicant: may be used interchangeably to represent a plant, a drug, marijuana, or chemical intoxicants such as cocaine, crystal meth, or opiates.

Chemical profile (chemovar): Chemical analysis for chemotaxonomic mapping of plant or microorganism varieties with differences in secondary metabolite content and distribution.

Genetic profile: Unique DNA pattern based on a set of sequence variations that differentiates individuals of a species, such as cannabis cultivars.

Cultivar (cultivated variety): The basic classification of a plant that is uniform and stable in its characteristics, can be reproduced toward defined goals and is not subject to extinction. Cultivar and intoxicant may be used interchangeably to represent a plant, a drug, marijuana, or chemical intoxicants such as cocaine, crystal meth, or opiates.

User profile: Personal data associated with a specific individual who may be a consumer, candidate or participant in a research study.

Method of consumption: Cannabis intake includes 3 routes: 1) inhalation; 2) ingestion and; 3) skin absorption. 1) Inhalation consumption forms are: a) smoking of combusted, dried flowers of the cannabis plant; b) vaporizing of cannabis flower or extract or concentrate in a precise temperature that allows therapeutic ingredients to phase-change into a gas or vapor and be extracted without burning the plant; and c) dabbing, the flash vaporization method of concentrates (shatter, wax, BHO, oil, etc.) which are more potent than cannabis flowers. 2) Ingestion: consumption of food-based edibles in solid or liquid form that have been formulated with cannabis-infused butter, infused oil or other cannabis-infused edibles such as candies, cookies and brownies. 3) Skin absorption: the route by which cannabis-infused topicals in the forms of lotions, balms, oils, lubricants and transdermal patches.

Demographic data: Socioeconomic characteristics of the tested users expressed statistically and includes categories such as: sex, age, race, income, education and employment. The population can be specific to geographic location and associated with time.

Metabolite profile/metabolic profile/metabolomics profile: Profile of pre-defined metabolites belonging to a class of compounds such as polar lipids, isoprenoids, carbohydrates, or members of particular pathways.

Metabolome: A complete set of metabolites that consists of low-molecular-weigh intermediates and products of the metabolism process in a biological system.

Metabolomics/metabolomic profiling: A global profiling process that measures multiple metabolite concentrations and fluctuations reflecting the dynamic response of a biological cell, tissue, organ or organism in response to drugs, diet, lifestyle, environment, stimuli and genetic modulations.

Metabolomics Data Acquisition: The process of comprehensive identification and quantification of a metabolite set from a sample of a biological system using the analytical platforms nuclear magnetic resonance (NMR) spectroscopy and or mass spectrometry (MS). To reduce sample complexity and to minimize ionization suppression effects, MS requires a previous separation step, using a hyphenated separation technique, such as gas chromatography (GC), high-performance liquid chromatography (HPLC) or ultra-performance liquid chromatography (UPLC), and capillary electrophoresis (CE).

Metabolomics Data Processing: The step in which the acquired raw data are submitted to an analytical platform for conversion into a numerical format that can be used for downstream statistical analysis. NMR data processing includes phasing, baseline correction, alignment, and normalization by software and algorithms, such as PERCH (PERCH Solution Ltd.), Chenomx NMR Suite (Chenomx Inc.), MestReNova (MestreLab Research), MetaboLab, AutoFit, TopSpin (Bruker Corp.), and MATLAB (The MathWorks Inc.). Hyphenated MS techniques data processing includes spectral deconvolution, dataset creation, grouping, alignment, filling data gaps, normalization, and data transformation using softwares such as XCMS, Mass Profiler Professional (MPP, Agilent Technologies), MZmine, MetAlign, MassLynx (Waters Corp.).

Metabolomics Statistical Analysis: The process that reveals discriminant metabolites between control and test samples using chemometric tools for sample overview and classification include: 1) multivariate analyses unsupervised methods, such as principal component analysis (PCA), and 2) supervised methods, such as partial least square discriminant analysis (PLS-DA) and orthogonal projections to latent structure discriminant analysis (OPLS-DA). Univariate analysis based on Student's t-test, Mann-Whitney U test, etc. can be used to confirm multivariate results.

Metabolite identification: The process that identifies putative metabolites and reveals the identity based on matching features from sample spectra against a reference spectral database and libraries, such as HMDB, KEGG, PubChem, Metlin, MassBank, LIPID MAPS, ChEBI, MMD, BioMagResBank, MetaboID, and Chenomx NMR Suite (Chenomx Inc.). Highly efficiency metabolite identification can be achieved by: (1) context-specific spectral database for biologically and biochemically possible candidates, and (2) incorporation of prior knowledge based on spectral dependencies, biochemical connectivities and biological relationships.

Machine learning, or alternatively described as machine learning database, or deep learning (as described below): Machine learning methods may include supervised learning, un-supervised learning, reinforcement learning, decision tree learning, association rule learning, artificial neural networks, deep learning, inductive logic programming, support vector machines, clustering, Bayesian networks, reinforcement learning, similarity and metric learning, genetic algorithms, rule-based machine learning, learning classifiers, recurrent neural networks, and adversarial neural networks.

Deep learning: May include neural networks and deep learning architectures such as deep neural networks, convolutional deep neural networks, deep belief networks and recurrent neural networks. Deep learning algorithms may use a cascade of many layers of nonlinear processing units for feature extraction and transformation. Each successive layer may use the output from the previous layer as input. The algorithms may be supervised or unsupervised. Deep learning algorithms may be based on the unsupervised learning of multiple levels of features or representations of the data.

Higher level features may be derived from lower level features to form a hierarchical representation. Deep learning algorithms may learn multiple levels of representations that correspond to different levels of abstraction; the levels form a hierarchy of concepts. Deep learning algorithms may comprise an output layer and one or more hidden layers, and training the deep learning algorithms may include: training the output layer by minimizing a loss function given the optimal set of assignments; and training the hidden layers through backpropagation.

Feature extraction: feature extraction starts from an initial set of measured data and builds derived values (features) intended to be informative and non-redundant, facilitating the subsequent learning and generalization steps, and in some cases leading to better human interpretations.

Time of consumption: includes unknown time and known time of consumption of an intoxicant or other consumable matter.

Confidence score: This specification generally describes a system that may train a machine learning algorithm that is configured to receive genetic and chemical profile of a cultivar, method of consumption and demographic data of a consumer, metabolomics data acquired at a time prior to consumption of said cultivar, at a time of consumption of said cultivar, and at a time after the consumption of said cultivar and estimate unknown time of consumption of an unknown cultivar and, for each estimate, generate a confidence score that represents the likelihood that a consumer consumed marijuana or another intoxicant within a specific time frame.

Disease state database/Medical indication database: The disease state database may comprise a database of diseases, metabolic profiles that indicate the disease is present in a patient, chemicals, toxins and other compounds found in the saliva that indicate a disease or toxicity, and corresponding treatment options for the disease. The diseases may include Alzheimer disease; anxiety; depression; pain; Parkinson; arthritis; cancer; neurodegenerative disorders; gastrointestinal diseases; presence of toxins; toxicity; food borne contaminations; toxicity by consuming pesticides used on marijuana plants; indications of consumption of pesticides and plant pesticide contaminants; indications of consumption of Shiga toxin-producing Escherichia coli, Salmonella spp, Aspergillus fumigatus, Aspergillus flavus, Aspergillus niger, Aspergillus terreus, Botrytis (mold) and Powdery Mildew; cognitive impairment; impairment of motor skills; glaucoma; high blood pressure; cardiovascular diseases; and mental health disorders. Plant Pesticide Contaminants may include, but are not limited to: Abamectin, Acephate, Acequinocyl, Acetamiprid, Aldicarb, Azoxystrobin, Bifenazate, Bifenthrin, Boscalid, Captan, Carbaryl, Carbofuran, Chlorantraniliprole, Chlordane, Chlorfenapyr, Chlorpyrifos, Clofentezine, Coumaphos, Cyfluthrin, Cypermethrin, Dam inozide, DDVP (Dichlorvos), Diazinon, Dimethoate, Dimethomorph, Ethoprop(hos), Etofenprox, Etoxazole, Fenhexamid, Fenoxycarb, Fenpyroximate, Fipronil, Flonicamid, Fludioxonil, Hexythiazox, Imazalil, Imidacloprid, Kresoxim-methyl, Malathion, Metalaxyl, Methiocarb, Methomyl, Methyl parathion, Mevinphos, Myclobutanil, Naled, Oxamyl, Paclobutrazol, Pentachloronitrobenzene, Permethrin, Phosmet, Piperonyl butoxide, Prallethrin, Propiconazole, Propoxur, Pyrethrins, Pyridaben, Spinetoram, Spinosad, Spiromesifen, Spirotetramat, Spiroxamine, Tebuconazole, Thiacloprid, Thiamethoxam and Trifloxystrobi. It will be appreciated by one skilled in the art that samples from a patient may include blood samples, urine samples, hair samples, other bodily fluids, other body tissues, or the breath of a patient.

In one embodiment, the proposed method may consist of the following collecting data and building database tables. In some embodiments, the data and database tables may comprise (1) medical indications and known metabolite profile and relevance of treatment; (2) patient drug treatment timeline metabolite tables; and (3) cultivar or source tables mapping cultivar (or strain) to known metabolites.

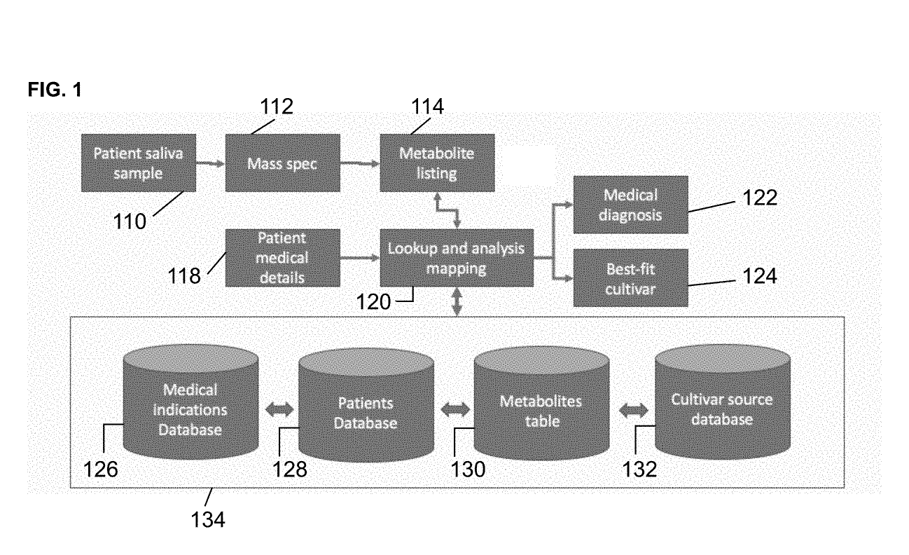

FIG. 1. illustrates a method or process for (1) screening a patient saliva sample 110 against a model (the model trained on a knowledge database 134 that combines medical indications 126, patient information in a patient database 128, metabolite tables 130, and cultivar source information 132) and (2) producing a recommended treatment or (3) producing a diagnosis. In one embodiment a patient saliva sample 110 is collected. In another embodiment, a mass spec 112 may be used for analysis of the patient saliva sample 110. A metabolite listing 114 may be produced to list the metabolites contained in the patient saliva sample 110. In other embodiments, the patient medical details 118 may be combined into the analysis 120 with the knowledge database to produce a medical diagnosis 122 and recommend a best-fit cultivar 124.

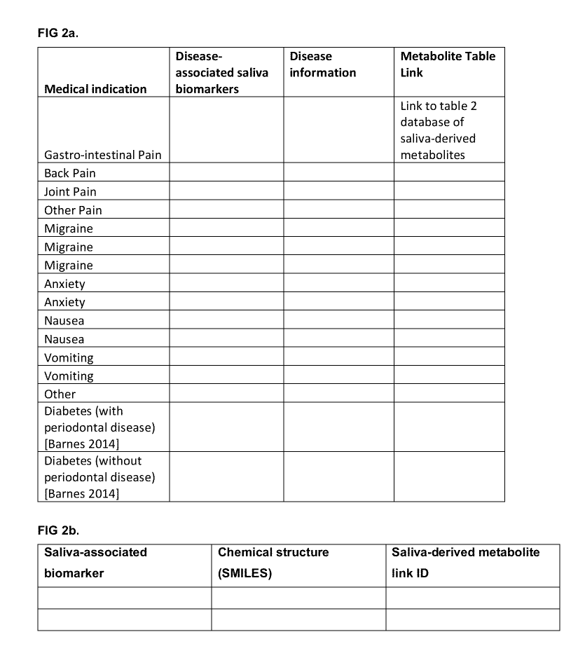

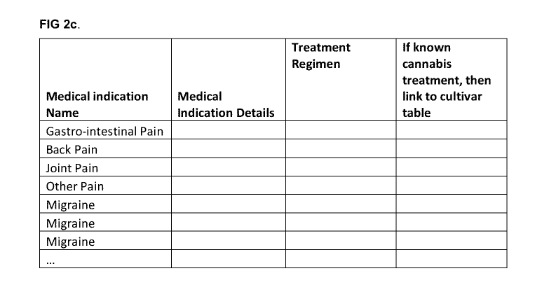

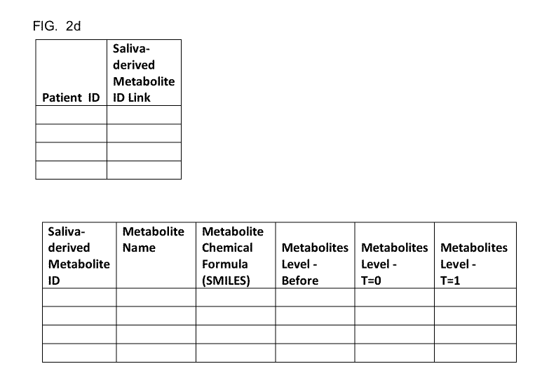

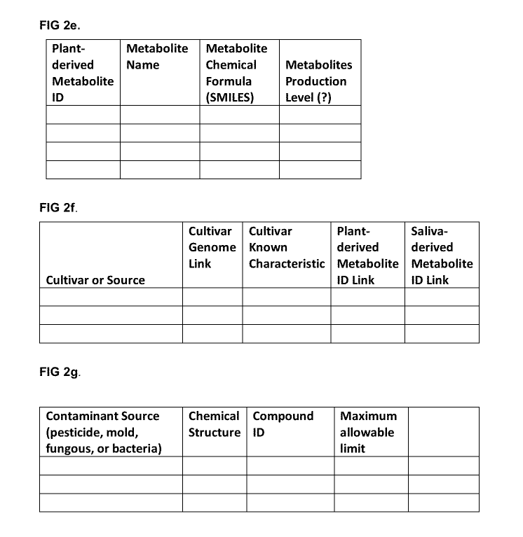

FIG. 2a shows a diagram illustrating features of a table including a plurality of records having a plurality of fields or columns that can be evaluated by a processing tool of the present subject matter. In some embodiments, the evaluation method may include an artificial intelligence method, a machine learning method, or a deep learning method. The processing tool may evaluate the table, such as one or more of the fields and assign a scoring measure to each of the evaluated fields. The scoring measure may identify the uniqueness in content contained within the associated field, correlation to other fields, tables, or databases.

FIG. 2b illustrates a diagram illustrating features of a table including a plurality of records having a plurality of fields or columns that can be evaluated by a processing tool of the present subject matter. In some embodiments, the evaluation method may include an artificial intelligence method, a machine learning method, or a deep learning method. The processing tool may evaluate the table, such as one or more of the fields and assign a scoring measure to each of the evaluated fields. The scoring measure may identify the uniqueness in content contained within the associated field, correlation to other fields, tables, or databases.

FIG. 2c illustrates a diagram illustrating features of a table including a plurality of records having a plurality of fields or columns that can be evaluated by a processing tool of the present subject matter. In some embodiments, the evaluation method may include an artificial intelligence method, a machine learning method, or a deep learning method. The processing tool may evaluate the table, such as one or more of the fields and assign a scoring measure to each of the evaluated fields. The scoring measure may identify the uniqueness in content contained within the associated field, correlation to other fields, tables, or databases.

FIG. 2d illustrates a diagram illustrating features of a table including a plurality of records having a plurality of fields or columns that can be evaluated by a processing tool of the present subject matter. In some embodiments, the evaluation method may include an artificial intelligence method, a machine learning method, or a deep learning method. The processing tool may evaluate the table, such as one or more of the fields and assign a scoring measure to each of the evaluated fields. The scoring measure may identify the uniqueness in content contained within the associated field, correlation to other fields, tables, or databases.

FIG. 2e illustrates a diagram illustrating features of a table including a plurality of records having a plurality of fields or columns that can be evaluated by a processing tool of the present subject matter. In some embodiments, the evaluation method may include an artificial intelligence method, a machine learning method, or a deep learning method. The processing tool may evaluate the table, such as one or more of the fields and assign a scoring measure to each of the evaluated fields. The scoring measure may identify the uniqueness in content contained within the associated field, correlation to other fields, tables, or databases.

FIG. 2f illustrates a diagram illustrating features of a table including a plurality of records having a plurality of fields or columns that can be evaluated by a processing tool of the present subject matter. In some embodiments, the evaluation method may include an artificial intelligence method, a machine learning method, or a deep learning method. The processing tool may evaluate the table, such as one or more of the fields and assign a scoring measure to each of the evaluated fields. The scoring measure may identify the uniqueness in content contained within the associated field, correlation to other fields, tables, or databases.

FIG. 2g illustrates a diagram illustrating features of a table including a plurality of records having a plurality of fields or columns that can be evaluated by a processing tool of the present subject matter. In some embodiments, the evaluation method may include an artificial intelligence method, a machine learning method, or a deep learning method. The processing tool may evaluate the table, such as one or more of the fields and assign a scoring measure to each of the evaluated fields. The scoring measure may identify the uniqueness in content contained within the associated field, correlation to other fields, tables, or databases.

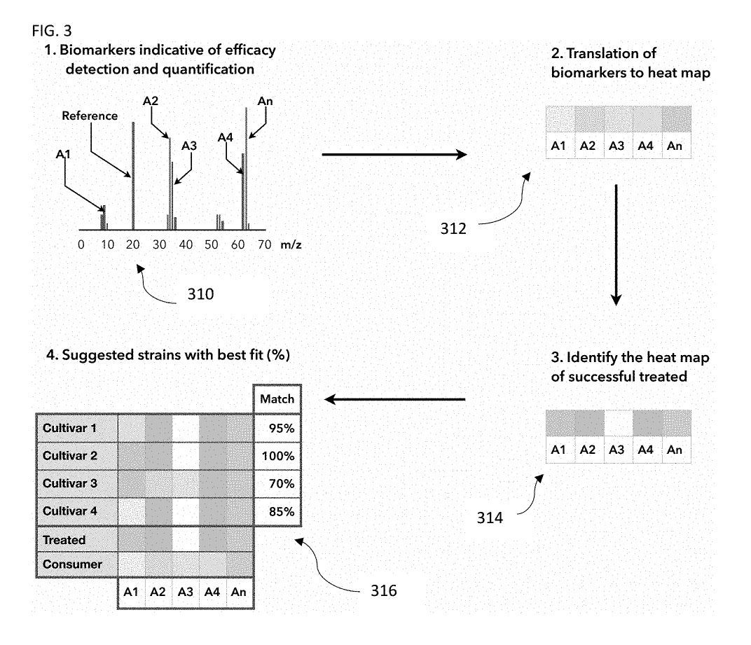

FIG. 3 illustrates a processing overview of the cultivar recommendation engine: (1) detection and quantification of biomarkers from a patient sample 310; (2) translation of the biomarkers panel to a heat map 312; (3) comparison with heat maps showing high efficacy treatment of patients with similar pharmacokinetics/demographic characteristics (age, gender and race) 314 and; (4) identification of high efficacy cultivars 316 based on the heat maps in (3).

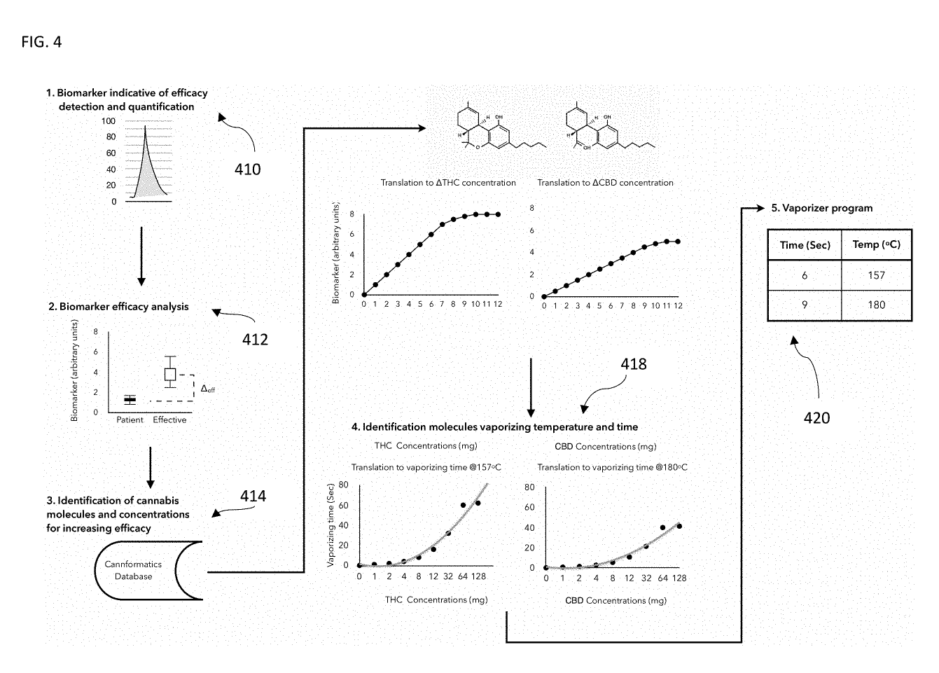

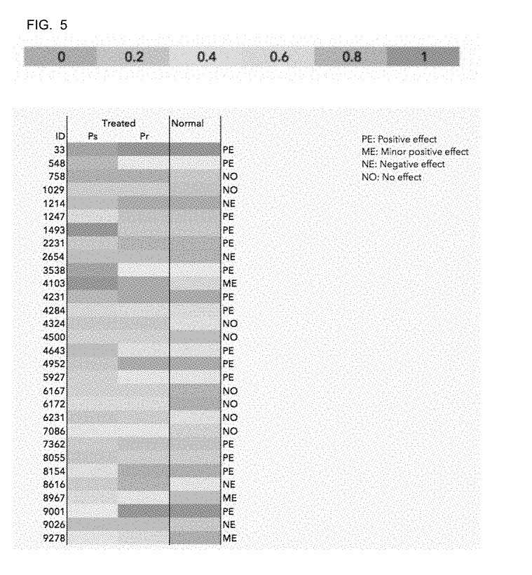

FIG. 4 Illustrates a processing overview of dosage recommendation engine: (1) detection and quantification (arbitrary units) of biomarkers from a patient sample 410; (2) comparison of the biomarker value with optimal value (high efficacy) of similar patients using the same cultivar 412; (3) identification of the relevant molecules and concentrations (mg) that contribute to the efficacy 414; (4) determination of the molecules vaporizing temperature (Table 13) and translation of the concentration (mg) of each molecule to vaporizing time 418 and; (5) adjustment of the vaporizer to the time and temperature accordingly 410.

FIG. 5 illustrates a heat map of biomarkers of efficacy values obtained from cannabis-treated patients for pain for pre (Pr) and Post (Ps) consumption, plus values for healthy individuals with similar consumer factors (e.g., age, gender, ethnicity, Body Mass Index).

In another embodiment, the method may generate fingerprints for patient cohorts in knowledge base tables. In other embodiments, artificial intelligence methods, machine learning methods, deep learning methods, or neural network methods may be used to generate fingerprints for patient cohorts in the knowledge base tables. In some embodiments, the method may include training a machine learning algorithm or neural net on three tables: (1) medical indications and known metabolite profile and relevance of treatment; (2) patient drug treatment timeline metabolite tables; and (3) cultivar or source tables mapping cultivar (or strain) to known metabolites; and (4) plant contaminants table listing potential chemical (e.g., insecticides, fungicides, metals) or biological contaminants (e.g., bacteria, fungi).

Some embodiments may include patient screening for determining appropriate cultivar and dosage including: collecting metabolite data from patient X (either one-time post dosing) or multiple hits before and post-dosing; generating patient metabolite fingerprint (optional); screening patient fingerprint or raw data against Knowledge Base using the machine learning or neural net model; deriving best matches for patient X metabolite pattern against current patient knowledge base; and recommending cultivar and dosage.

Other embodiments may include patient screening for discovering mitigation methods of a disease state (e.g., oxidative stress or increasing antioxidant status); this is desirable since diseases such as diabetes [Barnes 2014], multiple sclerosis [Karlik 2014], and systemic infections [Gutierrez 2013] result in increased oxidative stress and decreased antioxidant status. In some embodiments, mitigation methods of disease states may include: collecting metabolite data from patient X, either one-time post-dosing, or multiple hits before and post-dosing; generating a patient metabolite fingerprint (optional); screening a patient fingerprint or raw data against Knowledge Base using the machine learning or neural net model; deriving best matches for patient X metabolite pattern against the current patient knowledge base; and determining effect of dosing on disease state.

In alternative embodiments, rather than looking at mitigating the disease, one could use the above approach as a diagnostic without the treatment. It would be applicable to diseases including tuberculosis [Jacobs 2016], diabetes [Barnes 2014], multiple sclerosis [Karlik 2014], and systemic infections [Gutierrez 2013], which result in increased oxidative stress and decreased antioxidant status.

In alternative embodiments, rather than looking at mitigating the disease, one could use the above approach as a diagnostic for plant cultivar sources. It would be applicable to screening plant sources for contaminants such as (1) chemical additives (e.g., insecticides or fungicides) found during growth or processing, and (2) contaminants produced by bacteria or fungi or biological organisms themselves.

In some embodiments, data mining of the knowledge base may include a query for any patient/cultivar combinations that are very good or exceptional treatments; any data that strongly indicate a treatment has strong side effects; and any data that strongly indicate treatment is not effective.

In another embodiment, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for producing a recommended treatment, the method including receiving, by the one or more computing devices, a disease state database including the method also includes a disease state metabolite profile indicating a disease state. The method also includes a treatment regime for treating the disease state based on metabolite profile; receiving, by the one or more computing devices, a patient database including the method also includes a patient metabolite profile. The method also includes calculating, by the one or more computing devices, a correlation between the patient metabolite profile and the disease state metabolite profile. The method also includes generating, by the one or more computing devices, from the correlation between the patient metabolite profile and the disease state metabolite profile, a recommended treatment regime from the disease state database. Other embodiments of this aspect include corresponding computer systems, apparatus, and computer programs recorded on one or more computer storage devices, each configured to perform the actions of the methods.

Implementations may include one or more of the following features. The method for producing a recommended treatment where the disease state metabolite profile further includes metabolites available in saliva. The method for producing a recommended treatment where the patient metabolite profile further includes a pre-treatment patient metabolite profile. The method for producing a recommended treatment where the patient metabolite profile further includes a post treatment patient metabolite profile. The method for producing a recommended treatment where the patient metabolite profile further includes a plurality of post treatment patient metabolite profiles. The method for producing a recommended treatment further includes calculating a positive outcome score for the recommended treatment regime. The method for producing a recommended treatment where the calculating step includes an artificial intelligence method, a machine learning method or a deep learning method. The method for producing a recommended treatment further including obtaining a cultivar database including cultivar chemicals and metabolites. The method for producing a recommended treatment where the cultivar database further includes a metabolite response by a population of patients in response to consuming a cultivar. The method for producing a recommended treatment where the calculating step further includes calculating, by the one or more computing devices, a correlation between the patient metabolite profile, the cultivar database, and the disease state metabolite profile. The method for producing a recommended treatment further including predicting an alternative treatment regime. The method for producing a recommended treatment further including calculating a positive outcome score for the recommended treatment regime. Implementations of the described techniques may include hardware, a method or process, or computer software on a computer-accessible medium.

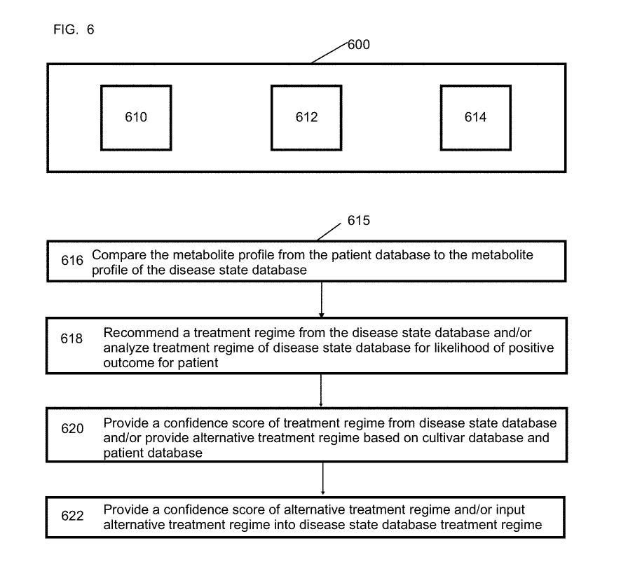

FIG. 6 illustrates a knowledge base 600 that may comprise a disease state database 610, a cultivar database 612, and a patient database 614. The disease state database 610 may comprise a metabolite profile and a treatment regime. The cultivar database 612 may comprise information about metabolites of chemicals from the cultivar, and metabolites produced in response to consuming a cultivar by a population of patients or by a single patient. The patient database 614 may comprise a metabolite profile. The metabolite profile may comprise data about metabolite profile of the patient pre-treatment, during treatment, and/or post treatment. In an alternative embodiment, the metabolite profile may comprise multiple post treatment metabolite profiles at multiple time points post treatment.

FIG. 6 further illustrates a flow chart 615 of a method which may be utilized in one or more embodiments. In one embodiment, the method may comprise comparing the metabolite profile from the patient database to the metabolite profile of the disease state database 616. In another embodiment the method may then comprise recommending a treatment regime from the disease state database and/or analyzing the treatment regime of the disease state database for a likelihood of positive outcome for the patient 618. In another embodiment, the method may comprise providing a confidence score of the treatment regime from the disease state database and/or providing an alternative treatment regime based on the cultivar database and the patient database 620. In another embodiment, the method may comprise providing a confidence score of the alternative treatment regime and/or inputting the alternative treatment regime into the disease state database under treatment regimes 622.

In one embodiment, the post treatment metabolite profiles may also be recorded into the cultivar database to provide data for metabolite response by a population of patients in response to consuming a cultivar or metabolite responses by individual patients in response to consuming a cultivar, and provide information about metabolites of chemicals produced by a cultivar.

In an alternative embodiment, the metabolite profile in the patient database may also be used as information in the cultivar database. In another embodiment, the metabolite profile from the patient database may be used to compare against the metabolite profile in the disease state database. This comparison will allow for a correlation to be examined or calculated to determine if there is a match between the metabolite profile of the disease state and the metabolite profile of the patient database. If there is a correlation, then the treatment regime that matches the disease state represented by the metabolite profile will be recommended as a treatment option for the patient. In an alternative embodiment, the comparison and correlation between the disease state database and the patient database will include information from the cultivar database. In one embodiment, if a match or correlation is made between the patient database and the disease state database, then the treatment regime from the disease state database that has been recommended will be analyzed for likelihood of positive outcome for the patient. This will utilize the cultivar database to determine the likelihood that a patient's metabolite profile will adjust to a non-disease state metabolite profile based on consuming the cultivar from the cultivar database.

In an alternative embodiment, the likelihood of positive outcome will utilize the cultivar database to determine if the recommended treatment will be able to alter the metabolite profile from the patient database to match a desired metabolite profile of a non-disease state or a desired metabolite profile.

In an alternative embodiment, if a match or correlation is made then an alternative treatment regime will be recommended based on the cultivar database and the likelihood of that cultivar having the desired reaction within the patient to result in a non-disease state. That will be determined by comparing the patient database metabolite profile with the disease state database metabolite profile and determining which cultivar inside the cultivar database will impact the metabolite profile of the patient database. This can be done by comparing the metabolites of chemicals from the cultivar or the metabolites produced in response to consuming the cultivar by a patient or population of patients and utilizing the difference between to give a recommend treatment option including a specific strain of cultivar, a specific dose, intervals of dosing, and methods of consumption.

In another embodiment, an alternative treatment regime will have a confidence score calculated and provided to the patient. In one embodiment, alternative treatment regimes calculated will be recorded back into the treatment regime of the disease state database.

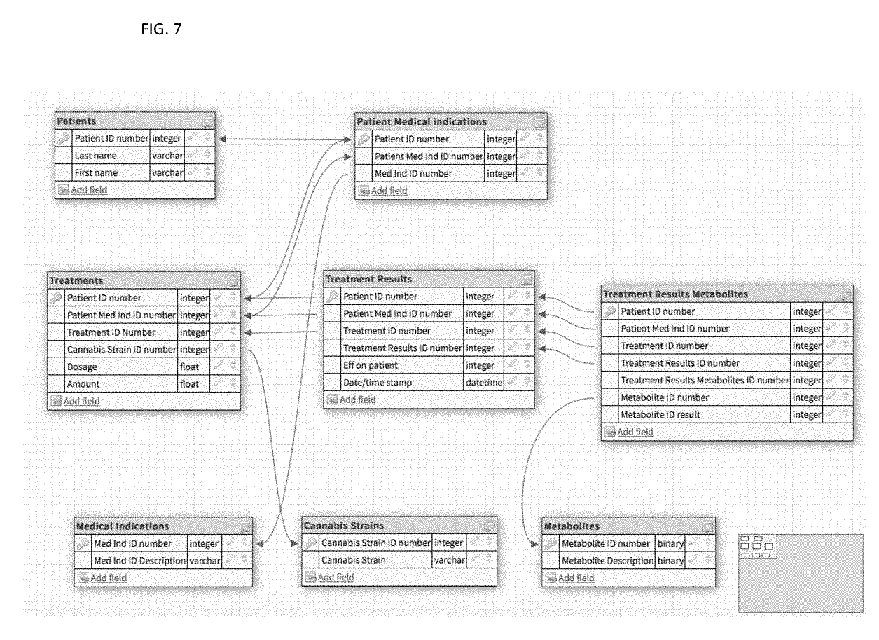

FIG. 7 capturing tables with links between them. This database schema may allow for the collection of data related to tracking patient medial indications, treatments and outcomes. These tables include the following: (1) Patients, which contains basic client information for each patient; (2) Patients Medical Indications, which identifies each treatment given to a patient; (3) Treatments, which records each treatment given to a patient; (4) Treatment Results, which records each result at various time points; (5) Treatment Results Metabolites, which identifies all of the metabolites and their relative amounts found in a sample. Other tables include Medical Indications, Cannabis Strains, and Metabolites.

In one embodiment, the patient's saliva is analyzed for biomarkers, chemicals, or metabolites that indicate the cultivar consumed by the patient was contaminated with pesticides and/or pesticide contaminants including but not limited to Abamectin, Acephate, Acequinocyl, Acetamiprid, Aldicarb, Azoxystrobin, Bifenazate, Bifenthrin, Boscalid, Captan, Carbaryl, Carbofuran, Chlorantraniliprole, Chlordane, Chlorfenapyr, Chlorpyrifos, Clofentezine, Coumaphos, Cyfluthrin, Cypermethrin, Dam inozide, DDVP (Dichlorvos), Diazinon, Dimethoate, Dimethomorph, Ethoprop(hos), Etofenprox, Etoxazole, Fenhexamid, Fenoxycarb, Fenpyroximate, Fipronil, Flonicamid, Fludioxonil, Hexythiazox, Imazalil, Imidacloprid, Kresoxim-methyl, Malathion, Metalaxyl, Methiocarb, Methomyl, Methyl parathion, Mevinphos, Myclobutanil, Naled, Oxamyl, Paclobutrazol, Pentachloronitrobenzene, Permethrin, Phosmet, Piperonyl butoxide, Prallethrin, Propiconazole, Propoxur, Pyrethrins, Pyridaben, Spinetoram, Spinosad, Spiromesifen, Spirotetramat, Spiroxamine, Tebuconazole, Thiacloprid, Thiamethoxam and/or Trifloxystrobi.

In other embodiments, the patient's saliva is analyzed for biomarkers, chemicals, or metabolites that indicate the cultivar consumed by the patient was contaminated with microorganisms including but not limited to Shiga toxin-producing Escherichia coli, Salmonella spp, Aspergillus fumigatus, Aspergillus flavus, Aspergillus niger, Aspergillus terreus, Botrytis (mold) and/or Powdery Mildew.

In one embodiment, the patient's saliva is analyzed for biomarkers, chemicals, or metabolites that indicate the patient consumed pesticides and/or pesticide contaminants including but not limited to Abamectin, Acephate, Acequinocyl, Acetamiprid, Aldicarb, Azoxystrobin, Bifenazate, Bifenthrin, Boscalid, Captan, Carbaryl, Carbofuran, Chlorantraniliprole, Chlordane, Chlorfenapyr, Chlorpyrifos, Clofentezine, Coumaphos, Cyfluthrin, Cypermethrin, Daminozide, DDVP (Dichlorvos), Diazinon, Dimethoate, Dimethomorph, Ethoprop(hos), Etofenprox, Etoxazole, Fenhexamid, Fenoxycarb, Fenpyroximate, Fipronil, Flonicamid, Fludioxonil, Hexythiazox, Imazalil, Imidacloprid, Kresoxim-methyl, Malathion, Metalaxyl, Methiocarb, Methomyl, Methyl parathion, Mevinphos, Myclobutanil, Naled, Oxamyl, Paclobutrazol, Pentachloronitrobenzene, Permethrin, Phosmet, Piperonyl butoxide, Prallethrin, Propiconazole, Propoxur, Pyrethrins, Pyridaben, Spinetoram, Spinosad, Spiromesifen, Spirotetramat, Spiroxamine, Tebuconazole, Thiacloprid, Thiamethoxam and/or Trifloxystrobi.

In other embodiments, the patient's saliva is analyzed for biomarkers, chemicals, or metabolites that indicate the patient consumed microorganisms including but not limited to Shiga toxin-producing Escherichia coli, Salmonella spp, Aspergillus fumigatus, Aspergillus flavus, Aspergillus niger, Aspergillus terreus, Botrytis (mold) and/or Powdery Mildew.

In some embodiments, the metabolites analyzed may include but are not limited to: (Compound Name The Human Metabolome Database (HMDB) Kyoto Encyclopedia of Genes and Genomes (KEGG) Formula) 15-HETrE (C20:3n6) HMDB10410 C20H30O5 Laurate (C12:0) DODECANOATE, RvE1 (C20:5n3) HMDB10410 C20H30O5 Linoleate (C18:2n6) LINOLEIC_ACID, Osbonate (C22:5n6) C22H34O2 Myristate (C14:0) CPD-7836, Nonadeca-10(Z)-enoate (C19:1 n9) HMDB13622 C19H36O2 Stearate (C18:0) 5-OXOHEXANOATE, 16(17)-EpDoPE (C22:6n3) C22H32O3 Arachidonate (C20:4n6) ARACHIDONIC_ACID, 15-HpETE (C20:4n6) HMDB04244 C05966 C20H32O4 PGD2 (C20:4n6) 5Z13E-15S-9-ALPHA15-DIHYDROXY-11-O, PGD2 (C20:4n6) HMDB01403 C00696 C20H32O5 20-hydroxy-LTB4 (C20:4n6) 20-OH-LTB4, LTB5 (C20:5n3) HMDB05073 C20H30O4 Timnodonate; EPA (C20:5n3) 5Z8Z11Z14Z17Z-EICOSAPENTAENOATE, Eicosenoate (C20:1 n9) HMDB02231 C16526 C20H38O2 Dihomo-_-linolenate (C20:3n6) CPD-8120, 12(13)-EpODE (C18:3n3) HMDB10200 C18H30O3 Bovinate (C18:2(9c,11t)-CLA) 9-CIS11-TRANS-OCTADECADIENOATE, 14,15-DiHETE (C20:5n3) HMDB10204 C20H32O4 13-HpODE (C18:2n6) 13-HYDROPEROXYOCTADECA-911-DIENOATE, OEA (C18:1 n9) HMDB02088 C20H39NO2 15-HETE (C20:4n6) CPD-1913, PGE3 (C20:5n3) HMDB02664 C06439 C20H30O5 AEA (C20:4n6) CPD-7598, 12(13)-EpOME (C18:2n6) HMDB04702 C18H32O3 12-HpETE (C20:4n6) 5Z8Z1 OE14Z-12S-12-HYDROPEROXYICOS, 12,13-DiHOME (C18:2n6) HMDB04705 C14829 C18H34O4 15-HpETE (C20:4n6) 5Z8Z11Z13E-15S-15-HYDROPEROXYICOS, 8(9)-EpETrE (C20:4n6) HMDB02232 C14769 C20H32O3 15-KETE (C20:4n6) 15-OXO-5811-CIS-13-TRANS-ICOSATETRAENO, 8,9-DiHETrE (C20:4n6) HMDB02311 C14773 C20H34O4 LEA (C18:2n6) CPD6666-4, 1-AG (C20:4n6) HMDB11578 C23H38O4 2-AG (C20:4n6) CPD-12600, α Linolenate (C18:3n3) HMDB01388 C18H30O2 25-(OH)D3 CALCIDIOL, 11(12)-EpETrE (C20:4n6) HMDB10409 C14770 C20H32O3 24R, 25-(OH)2D3 CPD-13032, 19,20-DiHDoPE (C22:6n3) HMDB10214 C22H34O4 LTB4 6Z8E10E14Z-512R-512-DIHYDROXYI, DEA (C22:4n6) HMDB13626 C13829 C24H41 NO2 PGE2 5Z13E-15S-1115-DIHYDROXY-9-OXOPROS, 5-HEPE (C20:5n3) HMDB05081 C20H30O3 PGD2 5Z13E-15S-9-ALPHA15-DIHYDROXY-11-O, 11-HETE (C20:4n6) HMDB04682 C20H32O3 20-OH-LTB4 20-OH-LTB4, DHEA (C22:6n3) C24H37NO2 15-HETE CPD-1913, 5,6-DiHETrE (C20:4n6) HMDB02343 C14772 C20H34O4 15-oxo-ETE 15-OXO-5811-CIS-13-TRANS-ICOSATETRAENO, PGE1 (C20:3n6) HMDB01442 C04741, D00180 C20H34O5 PGF2a 5Z13E-15S-9-ALPHA11-ALPHA15-TRIHY, 17(18)-EpETE (C20:5n3) HMDB10212 C20H30O3 Cortisol CORTISOL, aLEA (C18:3n3) HMDB13624 C20H35NO2 Aldosterone ALDOSTERONE, 15(16)-EpODE (C18:3n3) HMDB10206 C18H30O3 Pregnenolone PREGNENOLONE, 13-HODE (C18:2n6) HMDB04667 C14762 C18H32O3 Deoxycortisol 11-DEOXY-CORTISOL, Dihomo-γ-linolenate (C20:3n6) HMDB02925 C03242 C20H34O2 Corticosterone CORTICOSTERONE, NO-Gly (C18:1 n9) C20H37NO3 Progesterone PROGESTERONE, PEA (C16:0) HMDB02100 C16512 C18H37NO2 allopregnanolone 3-BETA-HYDROXY-5-ALPHA-PREGNANE-20-ONE, 2-LG (C18:2n6) HMDB11538 C21H38O4 2-methoxy-estradiol 2-METHOXY-ESTRADIOL-17B, Palmitate (C16:0) HMDB00220 C00249, D05341 C16H32O2 Estrone ESTRONE, Lipoxin A4 (C20:4n6) HMDB04385 C06314 C20H32O5 Estrone-Sulfate ESTRONE-SULFATE, Timnodonate; EPA (C20:5n3) HMDB01999 C06428 C20H30O2 Testosterone TESTOSTERONE, Bovinate (C18:2(9c,11t)-CLA) HMDB03797 C04056 C18H32O2 Estradiol CPD-352, 13-HpODE (C18:2n6) HMDB03871 C04717 C18H32O4 Androstenedione ANDROST4ENE, 9-HpODE (C18:2n6) HMDB06940 C18H32O4 2-hydroxy-estradiol 2-HYDROXY-ESTRADIOL, 13-KODE (C18:2n6) HMDB04668 C14765 C18H30O3 16_-hydroxy-estradiol ESTRIOL, Stearidonate (C18:4n3) HMDB06547 C18H28O2 2-oxobutyrate 2-OXOBUTANOATE, 15-HETE (C20:4n6) HMDB03876 C04742 C20H32O3 3-methyl-2-oxobutanoic acid (2-keto-isovalerate) 2-KETO-ISOVALERATE, 9(10)-EpODE (C18:3n3) HMDB10220 C18H30O3 glycolic acid GLYCOLLATE, 9-HETE (C20:4n6) HMDB10222 C20H32O3 estrone ESTRONE, Heptadecanoate (C17:0) HMDB02259 C17H34O2 1,3,5(10)-ESTRATRIEN-3,17_-DIOL (estradiol) CPD-352, PGD2 EA (PGD2) HMDB13629 C22H37NO5 estriol ESTRIOL, LTB4 (C20:4n6) HMDB02886 C05961 C20H34O6 oleic acid OLEATE-CPD, 6-keto-PGF1a (C20:4n6) HMDB02886 C05961 C20H34O6 palmitic acid PALMITATE, 5-HETE (C20:4n6) HMDB11134 C04805 C20H32O3 testosterone TESTOSTERONE, 13-HOTE (C18:3n3) HMDB10203 C18H30O3 pyruvic acid 2-DH-3-DO-D-ARABINONATE, LEA (C18:2n6) HMDB12252 C20H37NO2 succinic acid SUC, AEA (C20:4n6) HMDB04080 C11695 C22H37NO2 Epiestradiol CPD-351, 15,16-DiHODE (C18:3n3) HMDB10208 C18H32O4 adipic acid ADIPATE, 11,12-DiHETrE (C20:4n6) HMDB02314 C14774 C20H34O4 caprylic acid CPD-195, 9-KODE (C18:2n6) HMDB04669 C14766 C18H30O3 capric acid CPD-3617, Cervonate; DHA (C22:6n3) HMDB02183 C06429 C22H32O2 cholic acid CHOLATE, 12-HEPE (C20:5n3) HMDB10202 C20H30O3 lauric acid DODECANOATE, 9,12,13-TriHOME (C18:2n6) HMDB04708 C14833 C18H34O5 GLUTARIC ACID (Glutarate) GLUTARATE, 5-KETE (C20:4n6) HMDB10217 C14732 C20H30O3 linoleic acid LINOLEIC_ACID, 8-HETE (C20:4n6) HMDB04679 C14776 C20H32O3 malonic acid MALONATE, 20-HETE (C20:4n6) HMDB05998 C14748 C20H32O3 3-hydroxypropanoic acid 3-HYDROXY-PROPIONATE, 8,15-DiHETE (C20:4n6) HMDB10219 C20H32O4 4-hydroxybutyrate CPD-8575, 9-HODE (C18:2n6) HMDB10223 C18H32O3 azelaic acid CPD0-1265, 9,10,13-TriHOME (C18:2n6) HMDB04710 C14835 C18H34O5 myristic acid CPD-7836, RvD1 (C22:6n3) HMDB03733 C22H32O5 pentadecanoic acid CPD-8462, 19(20)-EpDoPE (C22:6n3) C22H32O3 stearic acid 5-OXOHEXANOATE, SEA (C18:0) HMDB13078; InChl=1/C20H41 NO2/c1-2-3-4-5-6-7-8-9-10-11-12-13-14-15-16-17-20(23)21-18-19-22/h22H, 2-19H2, 1H3,(H,21,23) C20H41 NO2 Pelargonic acid CPD-8505, LTE4 (C20:4n6) HMDB02200 C23H37NO5S_-Sitosterol CPD-4143, Linoleate (C18:2n6) HMDB00673 C01595 C18H32O2 pimelic acid CPD-205, Laurate (C12:0) HMDB00638 C02679 C12H24O2 (+)-4-cholesten-3-one CPD-323, PGB2 (C20:4n6) HMDB04236 C20H30O4 stigmasterol CPD-4162, 11,12,15 TriHETrE (C20:4n6) HMDB04684 C14782 C20H34O5 behenic acid DOCOSANOATE, 20-hydroxy-LTB4 (C20:4n6) HMDB01509 C04853 C20H32O5 arachidonic acid ARACHIDONIC_ACID, PGF2a (C20:4n6) HMDB01139 C20H34O5 octanal (Octyl Aldehyde) CPD-371, Stearate (C18:0) HMDB00827 C01530, D00119 C18H36O2 lanosterol LANOSTEROL, 9,10-DiHODE (C18:3n3) HMDB10221 C18H32O4 (R)-(+)-1,2-dithiolane-3-pentanoic acid (lipoic acid) LIPOIC-ACID, 20-HETE EA (20-HETE) HMDB13630 C22H37NO3 5-beta-cholestan-3-alpha-7-alpha-12-alpha-triol (3,7,12-Trihydroxycoprostane) 5-BETA-CHOLESTANE-3-ALPHA7-ALPHA12-ALP, 12(13)Ep-9-KODE (C18:2n6) HMDB13623; InChl=1/C18H30O4/c1-2-3-7-11-16-17(22-16)14-13-15(19)10-8-5-4-6-9-12-18(20)21/h13-14,16-17H, 2-12H2,1H3,(H,20,21)/b14-13+C18H30O4 L-norleucine L-2-AMINOHEXANOATE, 2-AG (C20:4n6) HMDB04666 C13856 C23H38O4 2-ketocaproic acid 2-OXOHEXANOATE, 12,13-DiHODE (C18:3n3) HMDB10201 C18H32O4 2-Ketovaleric acid CPD-3618, 14(15)-EpETE (C20:5n3) HMDB10205 C20H30O3 (−)-epicatechin CPD-7630, 9-HOTE (C18:3n3) C16326 C18H30O3 alpha tocopherol ALPHA-TOCOPHEROL, 1-OG (C18:1 n9) HMDB11567 C21H40O4 Lignoceric acid TETRACOSANOATE, Palmitoleate (C16:1 n7) HMDB03229 C16H30O2 arachidic acid ARACHIDIC_ACID, Palmitelaidate (C16:1 n7t) HMDB12328; HMDB03229 C16H30O2 oxalic acid OXALATE, Hepoxilin A3 (C20:4n6) HMDB04688 C20H32O4 naringenin NARINGENIN-CMPD, 17-HDoHE (C22:6n3) HMDB10213 C22H32O3 (+)-catechin CPD-1961, 20-carboxy-LTB4 (C20:4n6) HMDB06059 C05950 C20H30O6 cortisone CORTISONE, 14,15-DiHETrE (C20:4n6) HMDB02265 C14775 C20H34O4 5_-ANDROSTAN-17_-OL-3-ONE (dihydrotestosterone) 17-BETA-HYDROXY-5ALPHA-ANDROSTAN-3-O, Myristate (C14:0) HMDB00806 C06424 C14H28O2 phloretin PHLORETIN, δ 12-PGJ2 (C20:4n6) HMDB04238 C05958 C20H30O4 5-aminovaleric acid 5-AMINOPENTANOATE, Adrenate (C22:4n6) HMDB02226 C22H36O2 1-Hexadecanol CPD-348, DGLA EA (C20:3n6) HMDB13625 C13828 C22H39NO2 resveratrol CPD-83, Mead acid (C20:3n9) HMDB10378 C20H34O2 5beta-androstane-3,17-dione 5-BETA-ANDROSTANE-317-DIONE, 2-OG (C18:1n9) HMDB11537 C21H40O4 CHOLESTAN-3_,5_,6_-TRIOL (Cholestane-3,5,6-triol, (3.beta.,5.alpha.,6.beta.)-) CPD-8896, PGJ2 (C20:4n6) HMDB02710 C05957 C20H30O4 anandamide CPD-7598, Vaccenate (C18:1n7) HMDB03231 C08367 C18H34O2 phytosphingosine PHYTOSPINGOSINE, 5,15-DiHETE (C20:4n6) HMDB10216 C20H32O4 Zymosterol ZYMOSTEROL, 15-deoxy PGJ2 (C20:4n6) HMDB05079 C14717 C20H28O3 5-Dihydrocortisone [5_-PREGNAN-17,21-DIOL-3,11,20-TRIONE] CPD-287, 6-trans-LTB4 (C20:4n6) HMDB05087 C20H32O4 trans-trans-farnesol 2-TRANS6-TRANS-FARNESOL, γ-Linolenate (C18:3n6) HMDB03073 D07213,C06426 C18H30O2 dihydrolanosterol CPD-8606, 1-LG (C18:2n6) HMDB11568 C21H38O4 Dodecanol CPD-7867, 15-HEPE (C20:5n3) HMDB10209 C20H30O3 5-Cholesten-3-beta-ol=Cholesterol CHOLESTEROL, 14(15)-EpETrE (C20:4n6) HMDB04693 C14813 C20H32O4 androsterone ANDROSTERONE, Pentadecanoate (C15:0) HMDB00826 C16537 C15H30O2 squalene SQUALENE, PGF2a EA (PGF2a) HMDB13628 C22H39NO5 L-menthone CPD-1909, 9,10-DiHOME (C18:2n6) HMDB04704 C14828 C18H34O4 trans-dehydroandrosterone 3-BETA-HYDROXYANDROST-5-EN-17-ONE, 9(10)-EpOME (C18:2n6) HMDB04704 C14828 C18H34O4 lactic acid Lactate, PGE2 (C20:4n6) HMDB01220 C00584, D00079 C20H32O5 citral GERANIAL, TXB2 (C20:4n6) HMDB03252 C20H34O6 geraniol GERANIOL, 17,18-DiHETE (C20:5n3) HMDB10211 C20H32O4 ergosterol ERGOSTEROL, 15-KETE (C20:4n6) HMDB10210 C04577 C20H30O3 ALDOSTERONE ALDOSTERONE, NA-Gly (20:4n6) HMDB05096 C22H35NO3 norvaline L-2-AMINOPENTANOIC-ACID, Clupanodonate; DPA (C22:5n3) HMDB06528 C16513 C22H34O2 4-acetylbutyric acid HCN, Arachidonate (C20:4n6) HMDB01043 C00219 C20H32O2 alpha-santonin ALPHA-SANTONIN, 12-HpETE (C20:4n6) HMDB04243 C05965 C20H32O4 (_)-perillyl alcoholÃŠ CPD-261, 12-HETE (C20:4n6) HMDB06111 C20H32O3 10-Hydroxydecanoic acid 10-HYDROXYDECANOATE, Oleate (C18:1 n9) HMDB00207 C00712, D02315 C18H34O2 5-cholesten-3-beta-7-alpha-diol (7-alpha-Hydroxycholesterol)(7_-Hydroxycholesterol) CPD-266, PGD3 HMDB03034 C13802 C20H30O5 1,3,5(10)-estratrien-3,6-beta-17-beta-triol CPD-1077, Resolvin E1 C20H30O5 cholecalciferol VITAMIN_D3, allopregnanolone C21H34O2 2-hydroxybutanoic acid CPD-3564, 16(17)-EpDPE C22H32O3 abietic acidÃŠ CPD-8694, PGD2 HMDB01403 C00696 C20H32O5 6-hydroxy caproic acid CPD-102, LTB5 HMDB05073 C20H30O4 linolenic acid LINOLENIC_ACID, 14,15-DiHETE HMDB10204 C20H32O4 palmitoleic acid CPD-9245, PGE3 HMDB02664 C06439 C20H30O5 4,2′,4′-trihydroxychalcone CPD-3041, 12(13)-EpOME HMDB04702 C18H32O3 DEHYDROABIETIC ACID CPD-8725, PGD1 HMDB05102 C06438 C20H34O5 epigallocatechin CPD-10411, 12,13-DiHOME HMDB04705 C14829 C18H34O4 3-hydroxybutyric acid CPD-1843, 3alpha,5alpha-tetrahydrodeoxycorticosterone C21H34O3 heptadecanoic acid CPD-7830, 8(9)-EpETrE alt C20H32O3 1,3-diaminopropane CPD-313, 8(9)-EpETrE C20H32O3 2-Deoxyuridine DEOXYURIDINE, 13(14)-EpDPE C22H32O3 4-pyridoxic acid CPD-1112, 8,9-DiHETrE HMDB02311 C14773 C20H34O4 4-hydroxyphenylacetic acid 4-HYDROXYPHENYLACETATE, 11,12-DiHETE C20H32O4 3-ureidopropionate 3-UREIDO-PROPIONATE, 7,8-DiHDPE C22H34O4 biotin BIOTIN, 9(10)-EpOME HMDB04701 C18H32O3 adenine ADENINE, 24R, 25-(OH)2D3 C27H44O3 Adenosine 5′-monophosphate AMP, 19,20-DiHDPE HMDB10214 C22H34O4 melibiose MELIBIOSE, 5-HEPE HMDB05081 C20H30O3 adenosine ADENOSINE, 11-HETE HMDB04682 C20H32O3 cellobiose CELLOBIOSE, 5,6-DiHETrE HMDB02343 C14772 C20H34O4 Beta-alanine B-ALANINE, 1α,25-(OH)2D3 HMDB01903 C01673, D00129 C27H44O3 (−)-Adenosine 3′,5′-cyclic monophosphate CAMP, PGE1 HMDB01442 C04741, D00180 C20H34O5 (S)-Carnitine D-CARNITINE, 17(18)-EpETE C20H30O3 5,6-dihydrouracil DI—H-URACIL, 12-oxo-ETE C14807 C20H30O3 2′-deoxyguanosine DEOXYGUANOSINE, 15(16)-EpODE HMDB10206 C18H30O3 citric acid CIT, 13-HODE HMDB04667 C14762 C18H32O3 citric acid CIT, 15(S)-HETrE HMDB05045 C20H34O3 cytidine-5′-monophosphate CMP, LXA4 HMDB04385 C06314 C20H32O5 Homovanillic acid CPD-7651, 14(15)-EpETrE C20H32O3 glycine CPD-8569, 13-oxo-ODE HMDB04668 C14765 C18H30O3 D-(glycerol 1-phosphate) GLYCEROL-3P, 15-HETE HMDB03876 C04742 C20H32O3 glycocyamine (Guanidoacetic acid) GUANIDOACETIC_ACID, 9(10)-EpODE HMDB10220 C18H30O3 homogentisic acid HOMOGENTISATE, 25-(OH)D3 C01561 C27H44O2 glycerol GLYCEROL, Cortisol C00735, D00088 C21H30O5 guanosine GUANOSINE, 9-HETE HMDB10222 C20H32O3 fumaric acid FUM, 6-keto-PGF1a HMDB02886 C05961 C20H34O6 D-Glyceric acid GLYCERATE, 5-HETE HMDB11134 C04805 C20H32O3 L-Glutamic acid GLT, 25-(OH)D2 C28H44O2 Ethanolamine ETHANOL-AMINE, 13-HOTrE C18H30O3 D-(+)-Gluconic acid_-lactone GLC-D-LACTONE, 13,14-DiHDPE C22H34O4 gentisic acid CPD-633, 15,16-DiHODE HMDB10208 C18H32O4 L-Malic acid MAL, 11,12-DiHETrE HMDB02314 C14774 C20H34O4 hypoxanthine HYPOXANTHINE, 9-oxo-ODE HMDB04669 C14766 C18H30O3 L-tyrosine TYR, 12-HEPE HMDB10202 C20H30O3 L-proline PRO, 9,12,13-TriHOME HMDB04708 C14833 C18H34O5 L-threonine THR, 5-oxo-ETE HMDB10217 C14732 C20H30O3 L-Isoleucine ILE, 8-HETE C20H32O3 inosine 5′-monophosphate IMP, LTB3 C20H34O4 maleic acid MALEATE, 20-HETE HMDB05998 C14748 C20H32O3 L-histidine HIS, 8,15-DiHETE HMDB10219 C20H32O4 L-lysine LYS, 9-HODE HMDB10223 C18H32O3 lactose LACTOSE, 9,10,13-TriHOME C18H34O5 L-serine SER, 8,9-DiHETE C20H32O4 L-(+) lactic acid L-LACTATE, 10,11-DiHDPE C22H34O4 aspartic acid L-ASPARTATE, 7(8)-EpDPE C22H32O3 L-cystine CYSTINE, Corticosterone HMDB01547 C02140 C21H30O4 isocitric acid Isocitrate, Pregnenolone C01953, D00143 C21H32O2 inosine INOSINE, 19(20)-EpDPE C22H32O3 methylmalonic acid CPD-546, Ganaxolone C22H36O2 phenylpyruvate PHENYL-PYRUVATE, Aldosterone C01780 C21H28O5 N-epsilon-Acetyl-L-lysine CPD-567, PGB2 C05954 C20H30O4 alpha-ketoglutaric acid (2-Ketoglutaric acid) 2-KETOGLUTARATE, 20-OH-LTB4 HMDB01509 C04853 C20H32O5 PHENYLACETIC ACID PHENYLACETATE, PGF2a C20H34O5 L-ornithine L-ORNITHINE, 11(12)-EpETE C20H30O3 oxaloacetic acid OXALACETIC_ACID, 9,10-DiHODE HMDB10221 C18H32O4 O-phosphocolamine (O-phosphorylethanolamine) PHOSPHORYL-ETHANOLAMINE, EKODE C18H30O4 2-ketoadipate 2K-ADIPATE, 12,13-DiHODE HMDB10201 C18H32O4 orotic acid OROTATE, 14(15)-EpETE C20H30O3 quinolinic acid (Pyridine-2,3-dicarboxylic acid) QUINOLINATE, 9-HOTrE C16326 C18H30O3 pyridoxine PYRIDOXINE, Progesterone HMDB01830 C00410, D00066 C21H30O2 D-sorbitol SORBITOL, 17-HDoHE HMDB10213 C22H32O3 taurine TAURINE, 20-COOH-LTB4 HMDB06059 C05950 C20H30O6 sucrose SUCROSE, 14,15-DiHETrE HMDB02265 C14775 C20H34O4 thymine THYMINE, PGJ2 HMDB02710 C05957 C20H30O4 L-pyroglutamic acid (oxoproline) 5-OXOPROLINE, 5,15-DiHETE HMDB10216 C20H32O4 sarcosine SARCOSINE, 5(6)-EpETrE C20H32O3 O-phospho-L-serine (O-Phosphoserine) 3-P-SERINE, 15-deoxy-PGJ2 HMDB05079 C14717 C20H28O3 thymidine THYMIDINE, LTB4 HMDB01085 C02165 C20H32O4 L-Saccharopine SACCHAROPINE, d4-LTB4 C20H32O4 uric acid URATE, 6-trans-LTB4 HMDB05087 C20H32O4 urea UREA, THF diol C4H10O2 uridine URIDINE, Deoxycortisol C05488, D03595 C21H30O4 uracil URACIL, 15-HEPE C20H30O3 urocanic acid UROCANATE, 8-HEPE C20H30O3 tyramine TYRAMINE, 9,10-DiHOME HMDB04704 C14828 C18H34O4 DL-3,4-dihydroxyphenyl glycolÃŠÃŠ CPD-11878, PGE2 HMDB01220 C00584, D00079 C20H32O5 3-(3-hydroxyphenyl)propionic acid 3-HYDROXYPHENYL-PROPIONATE, d4-PGE2 C20H32O5 2,3-dihydroxybenzoic acid 2-3-DIHYDROXYBENZOATE, TXB2 HMDB03252 C20H34O6 3-hydroxyphenylacetic acid 3-HYDROXYPHENYLACETATE, d4-TXB2 C20H34O6 (S)-3-Hydroxybutyric acid CPD-1843, 17,18-DiHETE HMDB10211 C20H32O4 5-hydroxytryptophan 5-HYDROXY-TRYPTOPHAN, 15-oxo-ETE HMDB10210 C04577 C20H30O3 vanillic acid (4-hydroxy-3-methoxybenzoic acid) VANILLATE, 8(9)-EpETE C20H30O3 4-hydroxybenzoic acid 4-hydroxybenzoate, 16,17-DiHDPE C22H34O4 N-acetyl-L-phenylalanine CPD-439, 10(11)-EpDPE C22H32O3 creatinine CREATININE, 1α, 25-(OH)2D2 C28H44O3 cinnamic acid CPD-674, 12-HETE HMDB06111 C20H32O3 L-Cysteine CYS, 11,12-, 15-TriHETrE 2-FUROIC ACID 2-FUROATE, 5,6-DiHETE (chemspider 4444132) Glutaconic acid GLUTACONATE, 4,5-DiHDPE Cytosine CYTOSINE, 12(13)-EpODE citraconic acid 2-METHYLMALEATE, 11(12)-EpETrE L-glutamine GLN, d4-6-keto-PGF1a 3-indolelactic acid (Indolelactate) INDOLE_LACTATE, d11-14,15-DiHETrE L-kynurenine L-KYNURENINE, d6-20-HETE L-leucine LEU, d4-9-HODE 2-ketoisocaproic acid (alpha-ketoisocaproate) 2K-4CH3-PENTANOATE, d8-12-HETE L-methionine MET, d8-5-HETE mandelic acid CPD-122, d11-11(12)-EpETrE isoxanthopterin 2-AMINO-47-DIHYDROXYPTERIDINE, d4-9(10)-EpOME hippuric acid CPD-425, d8-AA 4-hydroxyquinoline-2-carboxylic acid (kynurenic acid) KYNURENATE, Androstenedione C19H26O2 pipecolic acid L-PIPECOLATE, 4-hydroxy-estradiol-2-glutatione L-homoserine HOMO-SER, 4-methoxy-estradiol C19H26O3 trans-4-hydroxy-L-proline 4-HYDROXY-L-PROLINE, 2-hydroxy-estrone-1-N-acetylcysteine 2-methylfumarate (mesaconate) MESACONATE, 2-methoxy-estradiol HMDB00405 C05302 C19H26O3 DL-p-hydroxyphenyllactic acidÃŠ 4-HYDROXYPHENYLLACTATE, 2-hydroxy-estrone-1-glutatione 5-hydroxyindole-3-acetic acidÃŠ 5-HYDROXYINDOLE_ACETATE, 2-hydroxy-estradiol C18H24O3 hydrocinnamic acid 3-PHENYLPROPIONATE, Estrone C18H22O2 D-mannitol MANNITOL, 4-hydroxy-estradiol-1-N-7-guanine phytanic acidÃŠ PHYTANATE, 2-hydroxy-estrone-6-N-3-adenine N-Acetyl-D-glucosamine N-ACETYL-D-GLUCOSAMINE, 2-hydroxy-estradiol-4-N-acetylcysteine N-acetyl-L-aspartic acid CPD-420, Estrone-Sulfate C18H22O5S phosphoglycolic acid CPD-67, 4-hydroxy-estradiol-2-N-acetylcysteine phenaceturic acid CPD-11715, 4-hydroxy-estrone-2-cysteine DL-4-hydroxymandelic acid 4-HYDROXYMANDELATE, 4-hydroxy-estrone-1-N-7-guanine C23H25N5O4 5alpha-cholestan-3-one CPD-1081, 4-hydroxy-estrone-1-N-3-adenine 4-methylcatechol 4-METHYLCATECHOL, 4-hydroxy-estradiol-1-N-3-adenine L-valine VAL, 2-OH-3-methoxy-estradiol L-citrulline CPD-7988, Testosterone C19H28O2 L-tryptophan TRP, 16α-hydroxy-estradiol C18H24O3 ferulic acid FERULIC-ACID, 4-methoxy-estrone C8H8.C7H12O2.C3H3N.C3H4O2 tartaric acid TARTRATE, 4-hydroxy-estradiol C18H24O3 catechol CATECHOL, 2-hydroxy-estradiol-1-glutatione C28H39N3O9S trans-aconitic acid CPD-225, 2-hydroxy-estradiol-6-N-3-adenine D-(+) trehalose (_{—,—}-Trehalose) TREHALOSE, 2-hydroxy-estrone C18H22O3 2′-Deoxycytidine 5′-triphosphate DCTP, 4-hydroxy-estradiol-2-cysteine Fructose 2,6-biphosphate CPD-535, 4-hydroxy-estrone-2-glutatione 2-amino-1-phenylethanolÃŠ PHENYLETHANOLAMINE, 2-hydroxy-estradiol-1+4-cysteine p-anisic acid CPD-1076, 2-hydroxy-estrone-1-cysteine trehalose-6-phosphate TREHALOSE-6P, 2-hydroxy-estradiol-4-glutatione C28H39N3O9S Aminomalonic acid AMINOMALONATE, Estradiol C18H24O2 4-aminophenol CPD-259, 2-methoxy-estrone C19H24O3 5_-deoxy-5_-(methylthio)adenosine (5′-methylthioadenosine) 5-METHYLTHIOADENOSINE, 4-hydroxy-estrone-2-N-acetylcysteine Allantoic acid ALLANTOATE, 16α-hydroxy-estrone HMDB00335 C18H22O3 thymidine 5′-monophosphate TMP, 2-hydroxy-estrone-4-cysteine 4-nitrophenol P-NITROPHENOL, 4-hydroxy-estrone C18H22O3 N-acetyl-5-hydroxytryptamine N-ACETYL-SEROTONIN, 2-OH-3-methoxy-estrone C19H24O3 flavin adenine dinucleotide FAD, 2-hydroxy-estrone-4-N-acetylcysteine acetanilide N-ACETYLARYLAMINE, 2-hydroxy-estradiol-1-N-acetylcysteine spermine SPERMINE, 2-hydroxy-estrone-4-glutatione succinate semialdehyde SUCC-S-ALD, quinic acid C7H12O6 4-nitrophenyl phosphate CPD-194, benzyl thiocyanate C02660 C8H7NS Guanosine 3′,5′-cyclic monophosphate CGMP, 5-Dihydrocortisol HMDB03259 C05471 C21H32O5 6-phosphogluconic acid CPD-2961, α-D-glucosamine phosphate C6H14NO8P 3,4-dihydroxyphenylacetic acidÃŠ CPD-782, 4-androsten-3,17-dione HMDB00053 C00280, D00051 C19H26O2 beta-hydroxypyruvate (3-hydroxypyruvate) OH-PYR, glycolic acid HMDB00115 C00160,C03547 C2H4O3 melatonin N-ACETYL-5-METHOXY-TRYPTAMINE, phytosphingosine HMDB04610 C12144 C18H39NO3 guanosine-5′-monophosphate GMP, N-methylaniline C02299 C7H9N guaiacol CPD-400, 2-hydroxybutyric acid HMDB00008 C4H8O3 nicotinamide (Nicotinacid-amide) NIACINAMIDE, 2-hydroxybutanoic acid C05984 C4H8O3 3beta-Hydroxy-5beta-pregnane-20-one CPD-5961, acenaphthenequinone C02807 C12H6O2 3-hydroxyanthranilic acid 3-HYDROXY-ANTHRANILATE, (R)-(+)-1,2-dithiolane-3-pentanoic acid (lipoic acid) HMDB01451 C16241 C8H14O2S2 nicotinic acid NIACINE, L-Isoleucine HMDB00172 C00407, D00065 C6H13NO2 pyridoxal 5_-phosphate PYRIDOXAL_PHOSPHATE, 3-aminopropionitrile HMDB04101 C05670 C3H6N2 N,N-dimethylarginine CPD-596, L-ornithine HMDB00214 C00077 C5H12N2O2 CORTICOSTERONE CORTICOSTERONE, hesperetin C16H14O6 benzoylformic acid PHENYLGLYOXYLATE, 3-hydroxypropanoic acid HMDB00700 C01013 C3H6O3 3-hydroxycinnamic acid (m-coumaric acid) CPD-10797, 4-aminobenzoic acid (p-Aminobenzoic acid) HMDB01392 C00568, D02456 C7H7NO2 progesterone PROGESTERONE, loganin C17H26O10 3,4-dihydroxybenzoic acid 3-4-DIHYDROXYBENZOATE, 2-METHYLGLUTARIC ACID HMDB00422 C6H10O4 p-cresol 24-DICHLOROPHENOL, neohesperidin C09806 C28H34O15 3,4-dihydroxymandelic acid CPD-11879, Tricetin C10192 C15H10O7 benzoic acid BENZOATE, 2-Deoxyerythritol C4H10O3 caffeic acid CPD-8098, phosphoglycolic acid HMDB00816 C00988 C2H5O6P 4-hydroxycinnamic acid (D-erythro-sphingosine) (p-Coumaric acid) COUMARATE, Acylcarnitine C18:1 HMDB05065 C25H47NO4 m-cresol CPD-112, LysoPC (16:0) HMDB10382 C24H50NO7P o-cresol CPD-109, beta-Hydroxymyristic acid C14H28O3 itaconic acid ITACONATE, 3beta-Hydroxy-5beta-pregnane-20-one HMDB01471 C11825 C21H34O2 phthalic acid PHTHALATE, 5p-PREGNAN-3β-OL-20-ONE C21H34O2 3-(4-hydroxyphenyl)propionic acid PHLORETATE, hydroxylamine HMDB03338 C00192 H3NO picolinic acid PICOLINATE, D-(glycerol 1-phosphate) HMDB00126 C00093 C3H9O6P 5,7-dihydroxy-3-(4-methoxyphenyl)chromen-4-one (Biochanin A=5,7-Dihydroxy-4′-Methoxyisoflavone) BIOCHANIN-A, glycerol 1-phosphate C03189 C3H9O6P Octadecanol CPD-7873, L-alpha-Glycerophophate C00623 C3H9O6P terephthalic acid TEREPHTHALATE, L-threonine HMDB00167 C00188, D00041 C4H9NO3 hydroquinone HYDROQUINONE, L-allothreonine HMDB04041 C05519 C4H9NO3 3-hydroxybenzoic acid 3-HYDROXYBENZOATE, threonine C4H9NO3_-glycerolphosphate (beta-Glycerophosphoric acid) CPD-536, 4-hydroxybenzyl cyanide C03766 C8H7NO 2-hydroxycinnamic acidÃŠ (o-coumaric acid) 2-COUMARATE, Gentiobiose C12H22O11 6-hydroxynicotinic acid 6-HYDROXY-NICOTINATE, p-benzoquinone HMDB03364 C00472 C6H4O2 1,5-Anhydroglucitol 15-ANHYDRO-D-GLUCITOL, β-glutamic acid C05574 C5H9NO4 4-nitrocatechol CPD-158, 2-Monopalmitin HMDB11533 C19H38O4 isomaltose CPD-1243, 3-methyloxyindole (3-Methyl-2-oxindole) C002366 C9H9NO 2-aminoethanethiol CPD-239, azelaic acid HMDB00784 C08261, D03034 C9H16O4 aniline ANILINE, 5-methoxypsoralen D07521,C01557 C12H8O4 shikimic acid SHIKIMATE, phenylethylamine C05332 C8H11N flavone CPD-8485, cholic acid HMDB00619 C00695 C24H40O5 benzyl alcohol BENZYL-ALCOHOL, 5-Hydroxyindole-2-carboxylic acid C9H7NO3 5-Aminoimidazole-4-carboxamide CPD-3762, 6-phosphogluconic acid HMDB01316 C00345 C6H13O10P daidzein DAIDZEIN, triacontanoic acid methyl ester C31H62O2 hydroxylamine HYDROXYLAMINE, cis-11-Eicosenoic acid HMDB02231 C16526 C20H38O2 N-acetyl-ornithine N-ALPHA-ACETYLORNITHINE, 3-hydroxy-L-proline C05147 C5H9NO3 p-benzoquinone P-BENZOQUINONE, L-Malic acid HMDB00156 C00149 C4H6O5 Tryptophol CPD-341, D-malic acid C00497 C4H6O5 L-gulonic acid_-lactone L-GULONO-1-4-LACTONE, sorbose C6H12O6 adenosine-3′-monophosphate (3′-adenylic acid) CPD-3706, D-tagatose HMDB03418 C6H12O6 butyraldehyde BUTANAL, L-sorbose (D-Psicose) C01452 C6H12O6 1-Methylhydantoin N-METHYLHYDANTOIN, fructose C10906, D00114 C6H12O6 4-acetamidobutyric acid CPD-35, dioctyl phthalate C03690 C24H38O4 5,6-dimethylbenzimidazole DIMETHYLBENZIMIDAZOLE, arbutin C06186 C12H16O7 2-mercaptoethanesulfonic acid CoM, 2-ketoisocaproic acid (alpha-ketoisocaproate) HMDB00695 C00233 C6H10O3 5-alpha-pregnan-3,20-dione (5-alpha-Dihydroprogesterone; 5_-PREGNAN-3,20-DIONE) CPD-293, 4-methyl-5-thiazoleethanol C04294 C6H9NOS DL-3-aminoisobutyric acid 3-AMINO-ISOBUTYRATE, 6-hydroxynicotinic acid HMDB02658 C01020 C6H5NO3 L-allothreonine L-ALLO-THREONINE, phytol (cis-Phytol) C20H40O4-vinylphenol CPD-1075, isopropyl β-D-1-thiogalactopyranoside C9H18O5S 5-METHOXYTRYPTAMINE (methoxytryptamine) CPD-12018, glycocyamine (Guanidoacetic acid) HMDB00128 C00581 C3H7N3O2 5-methoxy-3-indoleacetic acid CPD-12020, aspirin (O-acetylsalicylic acid) HMDB01879 C01405, D00109 C9H8O4 3-aminopropionitrile BETA-AMINOPROPIONITRILE, 4-aminobutyric acid (L-A-Amino-N-butyric acid) HMDB00112 C00334, D00058 C4H9NO2 D-threitol CPD-12825, 4-nitrophenol HMDB01232 C00870 C6H5NO3 pyrrole-2-carboxylic acid PYRROLE-2-CARBOXYLATE, 1-HYDROXYANTHRAQUINONE C02980 C14H8O3 N-gamma-acetyl-N-2-formyl-5-methoxykynurenamine (N-Acetyl-N-formyl-5-methoxykynurenamine) CPD-12022, phenylpyruvate HMDB00205 C00166 C9H8O3 2-(4-hydroxyphenyl)ethanolÃŠ CPD30-4151, TES C19H30O2 DL-Anabasine L-FUCOSE, 3-Cyanoalanine C4H6N2O2 kyotorphin CPD-210, β-cyano-L-alanine C02512 C4H6N2O2 gallic acid CPD-183, 1-Hexadecanol HMDB03424 C00823, D00099 C16H34O formononetin FORMONONETIN, N,N-dimethyl-1,4-phenylenediamine C04203 C8H12N2 phenylacetaldehyde PHENYLACETALDEHYDE, O-phospho-L-serine (O-Phosphoserine) HMDB00272 C01005 C3H8NO6P 3-indoleacetonitrile INDOLEYL-CPD, METHYL PHOSPHATE CH5O4P acetol ACETOL, lanosterol HMDB01251 C01724 C30H50O2-phenylacetamide CPD-238, Androstanediol C19H32O2 O-phospho-L-threonine L-THREONINE-O-3-PHOSPHATE, N-acetyl-L-phenylalanine HMDB00512 C03519 C11H13NO3 p-toluenesulfonic acid 4-TOLUENESULFONATE, 3-Hydroxypalmitic acid C16H32O3 4-hydroxybenzaldehyde 4-HYDROXYBENZALDEHYDE, methyl cinnamate C06358 C10OH10O2 1-Kestose 1-KESTOTRIOSE, allo-inositol (myo-inositol)(muco-Inositol) C6H12O6 melezitose (D-(+)-melezitose) CPD-13409, 2-aminophenol C01987 C6H7NO ciliatine CPD-1106, trans-3,5-dimethoxy-4-hydroxycinnamaldehyde C05610 C11H12O4 N-acetyl-L-leucine CPD-433, piceatannol HMDB04215 C05901 C14H12O4 4-methylumbelliferone CPD-182, 3,4-dihydroxyphenylacetic acid HMDB01336 C01161 C8H8O4 vanillin VANILLIN, DL-4-hydroxy-3-methoxymandelic acid (Vanillylmandelic acid) C05584 C9H10O5 Monoolein (1-Oleoyl-rac-glycerol) CPD-11690, Zymosterol HMDB06271 C05437 C27H44O1-Monopalmitin CPD-8508, 3-(2-hydroxyphenyl)propanoic acid C01198 C9H10O3 3-Hydroxypalmitic acid CPD-9781, N-benzyloxycarbonylglycine (Carbobenzyloxyglycine) C03710 C10H11NO4 beta-Hydroxymyristic acid KDO, hexachlorobenzene C11042 C6Cl6 pyrophosphate PPI, aspartic acid HMDB00191 C00049, D00013 C4H7NO4 sulfuric acid SULFATE, D-Aspartic acid C16433 C4H7NO4 Glucose-1-phosphate GLC-1-P, 4-isopropylbenzoic acid (CUMIC ACID) C06578 C10H12O2 galactose D-Galactose, 2,5-dihydroxybenzaldehyde HMDB04062 C05585 C7H6O3 ribulose-5-phosphate RIBULOSE-5P, phenylphosphoric acid C02734 C6H7O4P L-dithiothreitol DITHIOTHREITOL, PE (22:6(4Z,7Z,10Z,13Z,16Z,19Z)/16:0) HMDB09682 C43H74NO8P L-carnitine CARNITINE, 5-methoxy-3-indoleacetic acid HMDB04096 C05660 C11H11NO3 3-Indolepyruvic acid INDOLE_PYRUVATE, phenylalanine C02057 C9H11NO2 xanthine XANTHINE, phlorobenzophenone (2,4,6-Trihydroxybenzophenone) C06356 C13H10O4 alpha-Ecdysone ECDYSONE, PC(P-16:0/18:1 (9Z)) HMDB11210 C42H82NO7P 3,5-dimethoxy-4-hydroxycinnamic acid (sinapinic acid) SINAPATE, farnesol C15H26O D-malic acid CPD-660, trans-trans-farnesol HMDB06835,HMDB04305 C01126,C06081 C15H26O L-cysteic acid L-CYSTEATE, D-(+)-Ribonic acid gamma-lactone HMDB01900 C5H8O5 mandelonitrile MANDELONITRILE, Creatine HMDB00064 C00300 C4H9N3O2 cyclohexylamine (cyclohexanamine) CPD-303, chlorogenic acid C16H18O9 coniferyl alcoholÃŠ CONIFERYL-ALCOHOL, taxifolin C01617 C15H12O7 L-alpha-Glycerophophate SN-GLYCEROL-1-PHOSPHATE, (+/−)-Taxifolin C15H12O7 N-acetyl-L-glutamic acid ACETYL-GLU, N-methyltryptophan C12H14N2O2 4-hydroxymandelonitrile CPD-12889, N-2-fluorenylacetamide C02778 C15H13NO serine SER, sucrose HMDB00258 C00089, D00025, D06528, D06529, D06530, D06531, D06533 C12H22O11 orcinol (5-METHYLRESORCINOL) ORCINOL-CPD, hexadecane C16H34 L-cysteine CYS, L-asparagine C16438 C4H8N2O3 flavanone FLAVANONES, 1,4-Cyclohexanedione C08063 C6H8O2 gluconic acid 11-DEOXYCORTICOSTERONE, creatinine HMDB00562 C00791, D03600 C4H7N3O tryptophan TRP, beta-Mannosylglycerate C11544 C9H16O9 naphthalene NAPHTHALENE, Ala-Ala C6H12N2O3 pantothenic acid PANTOTHENATE, D-Ala-D-Ala C6H12N2O3 D,L-Tartaric acid TARTRATE, nicotinamide (Nicotinacid-amide) HMDB01406 C00153, D00036 C6H6N2O 1,4-dithioerythritol DITHIOERYTHRITOL, Acylcarnitine C18:3 C25H43NO4 3-indoleacetic acid INDOLE_ACETATE_AUXIN, β-glycerolphosphate (beta-Glycerophosphoric acid) HMDB02520 C02979 C3H9O6P N-Methyl-L-glutamic acid CPD-404, glycine-d5 deuterated C2H5NO2 1,3-Cyclohexanedione CYCLOHEXANE-13-DIONE, glycine HMDB00123 C00037, D00011 C2H5NO2 N-Acetyl-beta-alanine CPD-580, mono(2-ethylhexyl)phthalate C03343 C16H22O4 pyrogallol PYROGALLOL, 4-hydroxy-3-methoxycinnamaldehyde C02666 C10H10O3 O-succinylhomoserine O-SUCCINYL-L-HOMOSERINE, 2-Butyne-1,4-diol C02497 C4H6O2 N-Acetyl-D-galactosamine CPD-3604, prunin C09099 C21H22O10 3-(1-Pyrazolyl)-L-alanine CPD-670, melibiose HMDB00048 C05402 C12H22O11 4-hydroxyphenylpyruvic acid P-HYDROXY-PHENYLPYRUVATE, isomaltose HMDB02923 C00252 C12H22O11 3-(2-hydroxyphenyl)propanoic acid MELILOTATE, Melibiose C12H22O11 1-Aminocyclopropanecarboxylic acid CPD-68, 3-indoleacetonitrile HMDB06524 C02938 C10H8N2 benzoin Benzoin, ergosterol C01694 C28H44O oxamic acid OXAMATE, estrone HMDB00145 C00468, D00067 C18H22O2 L-sorbose (D-Psicose) SORBOSE, PC(20:4(5Z,8Z,11Z,14Z)/18:0) HMDB08431 C46H84NO8P alizarin ALIZARIN, PE(18:2(9Z,12Z)/18:1(9Z)) HMDB09092 C41H76NO8P 3-hydroxyflavone CPD-3261, melatonin HMDB01389 C01598 C₁₃H₁₆N₂O₂ 1-Indanone 1-INDANONE, uridine HMDB00296 C00299 C9H12N2O6 maleamic acid MALEAMATE, D-saccharic acid HMDB00663 C6H10O8 taxifolinÃŠ CPD-474, N-epsilon-Acetyl-L-lysine HMDB00206 C02727 C8H16N2O3 aldohexose (generic) ALTROSE, linolenic acid C06427 C18H30O2 1-INDANOL 1-INDANOL, phenylacetaldehyde HMDB06236 C0601 C8H8O7,8-dimethylalloxazine CPD-605, PE(20:4(5Z,8Z,11Z,14Z)/16:0) HMDB09385 C41H74NO8P methionine MET, 5-hydroxyindole-3-acetic acid HMDB00763 C05635 C10H9NO3 palatinose CPD-230, 5-Aminoimidazole-4-carboxamide HMDB03192 C04051 C4H6N4O resorcinol CPD-623, PE(18:1(9Z)/16:0) HMDB09055 C39H76NO8P scopoletin SCOPOLETIN, Leucrose C12H22O11 albendazole ALBENDAZOLE, 4,2′,4′-trihydroxychalcone C08650 C15H12O4 L-homocystine HOMOCYSTINE, cycloserine C06682 C3H6N2O2 xanthotoxin CPD-13042, 2-Carboxybenzaldehyde C03057 C8H6O3 maltose MALTOSE, 2-Deoxytetronic acid HMDB00337 C4H8O4 2-aminophenol 2-AMINOPHENOL, tyramine HMDB00306 C00483 C8H11NO phenylalanine 2-AMINO-3-3-OXOPROP-2-ENYL-BUT-2-ENEDI, 9-phenanthrenol C011430 C14H10O acetylisatin (N-Acetylisatin)N-ACETYLISATIN, N-acetyl-D-tryptophan C03137 C13H14N2O3 phloroglucinol CPD-16, benzoylformic acid HMDB01587 C02137 C8H6O3 3,4-dimethoxybenzaldehyde VERATRALDEHYDE, D-sorbitol HMDB00247 D00096 C6H14O6 Digalacturonic acid CPD-12435, D-mannitol HMDB00765 D00062 C6H14O6 dihydrocoumarin DIHYDROCOUMARIN, Pelargonic acid HMDB00847 C01601 C9H18O2 tartronic acid HYDROXYMALONATE, lipoamide (Thioctamide) HMDB00962 C00248, D00048 C8H15NOS2 N-methylaniline N-METHYLANILINE, hypoxanthine HMDB00157 C00262 C5H4N4O sinapyl alcohol SINAPYL-ALCOHOL, tartaric acid HMDB00956 D00103 C4H6O6 3-methyloxyindole (3-Methyl-2-oxindole) 3-METHYLOXINDOLE, D,L-Tartaric acid C00898 C4H6O6 2-Butyne-1,4-diol 2-BUTYNE-14-DIOL, L-methionine HMDB00696 C00073, D00019 C5H11NO2S 2-Hydroxybiphenyl CPD-946, methionine C01733, D04983 C5H11NO2S 2-hydroxypyridine 2-HYDROXYPYRIDINE, 2-ketoadipate HMDB00225 C00322 C6H8O5 _-cyano-L-alanine CPD-603, PE(P-18:0/22:6(4Z,7Z,10Z,13Z,16Z,19Z)) HMDB11394 C45H78NO7P 2,3-dihydroxybiphenyl (3-phenylcatechol) BIPHENYL-23-DIOL, 4-hydroxybenzoic acid HMDB00500 C00156 C7H6O3 sucrose-6-phosphate SUCROSE-6P, PE(20:4(5Z,8Z,11Z,14Z)/18:0) HMDB09387 C43H78NO8P citramalic acid CPD-31, hexadecanoic acid methyl ester (methyl hexadecanoate) C16995 C17H34O2 benzyl thiocyanate SELENATE, Dehydroepiandrosterone C19H28O2 4-hydroxy-3-methoxycinnamaldehyde CONIFERYL-ALDEHYDE, trans-dehydroandrosterone C01227 C19H28O2 indole-3-acetamide CPD-237, Acylcarnitine C18:0 C25H49NO4 phenylphosphoric acid PHENOL-PHOSPHATE, ribulose-5-phosphate C00199 C5H11O8P N-2-fluorenylacetamide 2-ACETAMIDOFLUORENE, L-canavanine C5H12N4O3 acenaphthenequinone ACENAPHTHENEQUINONE, Maleamate C4H5NO3 benzene-1,2,4-triol CPD-8130, maleamic acid C01596 C4H5NO3 cyclohexylsulfamic acid CPD-1124, sarcosine HMDB00271 C0213 C3H7NO2 3-methylcatechol CPD-111, naringenin HMDB02670 C00509 C15H12O5 3,4-Dihydroxypyridine 3-HYDROXY-4H-PYRID-4-ONE, 4-vinylphenol HMDB04072 C05627 C8H8O (+)-6-aminopenicillanic acid 6-AMINOPENICILLANATE, Acylcarnitine C12:0 HMDB02250 C19H37NO4 1-HYDROXYANTHRAQUINONE CPD-3301, liquiritigenin (4′,7-dihyroxyflavanone) C15H12O4 L-methionine sulfoxide L-Methionine-sulfoxides, 3-hydroxyphenylacetic acid HMDB00440 C05593 C8H8O3 N-methylanthranilic acid CPD-402, PE(22:6(4Z,7Z,10Z,13Z,16Z,19Z)/18:1(9Z)) HMDB09686 C45H76NO8P 2,4-dihydroxypyrimidine-5-carboxylic acid (uracil-5-carboxylic acid) CPD-629, O-methylthreonine C5H11NO3 2-Carboxybenzaldehyde CPD-1131, maltotriose C18H32O16 3-hydroxybenzaldehyde 3-OH-BENZALDEHYDE, acetanilide HMDB01250 C07565 C8H9NO phenyl-beta-glucopyranoside (Phenyl beta-D-glucopyranoside) CPD0-1667, S-carboxymethylcysteine C03727, D00175 C5H9NO4S N-acetyl-D-tryptophan N-ACETYL-D-TRYPTOPHAN, 4-hydroxypyridine C5H5NO N-formyl-L-methionine N-FORMYLMETHIONINE, N-Methyl-DL-alanine C4H9NO2 glycerol 1-phosphate GLYCEROL-3P, Methyl jasmonate (methyljasmonate) C13H20O3 1-Hydroxy-2-naphthoic acid HYDROXYNAPHTHOATE, adenine HMDB00034 C00147, D00034 C5H5N5 lumazine 24-DIHYDROXYPTERIDINE, 1-Methyladenosine C11H15N5O4 D-glucose-6-phosphate D-glucose-6-phosphate, benzene-1,2,4-triol C02814 C6H6O3 mono(2-ethylhexyl)phthalate 2-ETHYLHEXYL-PHTHALATE, 2,3-DIHYDROXYPYRIDINE C5H5NO2 3-hydroxybenzyl alcohol 3-OH-BENZYL-ALCOHOL, resorcinol C01751, D00133 C6H6O2 3-Methylthiopropylamine (3-(Methylthio)-propylamine) CPD-631, pantothenic acid C00864 C9H17NO5 tyrosine methyl ester L-TYROSINE-METHYL-ESTER, capric acid HMDB00511 C01571 C10H20O2 farnesal 2-TRANS6-TRANS-FARNESAL, quinolinic acid (Pyridine-2,3-dicarboxylic acid) HMDB00232 C03722 C7H5NO4 4-nitroquinoline N-oxide 4-NITROQUINOLINE-N-OXIDE, Octadecanol HMDB02350 D01924 C18H38O1,4-Dihydroxy-2-naphthoic acid DIHYDROXYNAPHTHOATE, 2,3-dihydroxybenzoic acid HMDB00397 C00196 C7H6O4 dioctyl phthalate BIS2-ETHYLHEXYLPHTHALATE, isoxanthopterin HMDB00704 C03975 C6H5N5O2 N-benzyloxycarbonylglycine (Carbobenzyloxyglycine) CPD-214, geraniol C01500 C10H18O trans-cyclohexane-1,2-diol TRANS-CYCLOHEXANE-12-DIOL, 2,6-Diaminopimelic acid C7H14N2O4 4-hydroxybenzyl cyanide CPD-1074, 0-succinylhomoserine C01118 C8H13NO6 piperine N-EE-PIPEROYL-PIPERIDINE, 1,2-Didecanoylglycerol C23H44O5 diethyl oxalpropionate (2,8-Dihydroxyquinoline) DIETHYL-2-METHYL-3-OXOSUCCINATE, 2,2-DIMETHYLSUCCINIC ACID HMDB02074 C6H10O4 N,N-dimethyl-1,4-phenylenediamine CPD0-1148, Diglycerol C6H14O5 N, N-dimethyl-L-histidine (N(alpha), N(alpha)-dimethyl-L-histidine) NALPHANALPHA-DIMETHYL-L-HISTIDINE, PC(16:1 (9Z)/16:1 (9Z)) HMDB08002 C40H76NO8P 4-methyl-5-thiazoleethanolÃŠTHZ, 3-HYDROXYPYRIDINE C5H5NO cis-1,2-dihydro-1,2-naphthalenediol ((1R,2S)-cis-1,2-dihydro-1,2-naphthalenediol) CIS-12-DIHYDRONAPHTHALENE-12-DIOL, inosine 5′-monophosphate HMDB00175 C00130 C10H13N4O8P N-carbobenzyloxy-L-leucine CPD-199, D-Fructose 6-phosphate HMDB00124 C6H13O9P 3-isopropylmalate 2-D-THREO-HYDROXY-3-CARBOXY-ISOCAPROATE, benzylsuccinic acid (DL-Benzylsuccinic acid) C11H12O4 3-hydroxy-L-proline CPD-664, trans-aconitic acid HMDB00958 C02341 C6H6O6 Nicotianamine CPD-463, cellobiose HMDB00055 C06422 C12H22O11 phenylethylamine PHENYLETHYLAMINE, maltose C01971 C12H22O11 DL-4-hydroxy-3-methoxymandelic acid (Vanillylmandelic acid) CPD-926, lactose HMDB00186 C00243, D00046 C12H22O11 trans-3,5-dimethoxy-4-hydroxycinnamaldehyde SINAPALDEHYDE, alpha tocopherol HMDB01893 C02477 C29H50O2 cis-o-coumarinic acid CPD-7418, adipamide C6H12N2O2 1,2-CYCLOHEXANEDIONE CPD0-1349, thymidine 5′-monophosphate HMDB01227 C00364 C10H15N2O8P arbutin HYDROQUINONE-O-BETA-D-GLUCOPYRANOSIDE, D (+)altrose C6H12O6 salicylaldehyde SALICYLALDEHYDE, D-allose (allose) C6H12O6 4-hydroxy-3-methoxybenzyl alcohol VANILLYL-ALCOHOL, D-(+)-Glucose HMDB00122 C6H12O6 aniline-o-sulfonic acid 2-AMINOBENZENESULFONATE, aldohexose (generic) C01662 C6H12O6 2-Hydroxyquinoline QUINOLIN-2-OL, N-ethylmaleamic acid C6H9NO3 4-hydroxyquinoline QUINOLIN-4-OL, cyclohexylsulfamic acid C02824, D02442 C6H13NO3S phlorobenzophenone (2,4,6-Trihydroxybenzophenone) CPD-6881, cis-4-hydroxycyclohexanecarboxylic acid HMDB01988 C7H12O3 methyl cinnamate CPD-6421, stigmasterol HMDB00937 C05442 C29H48O4-quinolinecarboxylic acid QUINOLINE-4-CARBOXYLATE, 4-hydroxyquinoline-2-carboxylic acid (kynurenic acid) HMDB00715 C01717 C10H7NO3 D-Tyrosine D-TYROSINE, D-(+) trehalose (α,α-Trehalose) HMDB00975 C01083 C12H22O11 atrazin-2-hydroxy HYDROXYATRAZINE, xylitol HMDB02917 C5H12O5 biuret CPD-809, D-arabitol C5H12O5 4-isopropylbenzyl alcohol (cuminic alcohol) CPD-1002, ribitol (Adonitol) HMDB00568 C00379,C00474,C01904,D00061 C5H12O5 biphenyl BIPHENYL, beta-hydroxypyruvate (3-hydroxypyruvate) HMDB01352 C00168 C3H4O4_-caprolactam CPD-883, 1,3-Cyclohexanedione C01066 C6H8O2 paraoxon ethyl PARAOXON, formononetin HMDB05808 C00858 C16H12O4 cycloserine CPD-2483, carbamoyl-aspartate C5H8N2O5 9-fluorenone 9FLUORENONE, L-histidine HMDB00177 C00135, D00032 C6H9N3O2 4-Methylbenzyl alcohol 4-METHYLBENZYL-ALCOHOL, citraconic acid HMDB00634 C02226 C5H6O4 hydroxyurea HYDROXY-UREA, 2-methylfumarate (mesaconate) HMDB00749 C01732 C5H6O4 lactulose CPD-3561, 4-hydroxymandelonitrile C00650 C8H7NO2 ACETOPHENONE PHENYL, 3,5-dihydroxyphenylglycine (1α-2S-Dihydroxy Phenylglycine) C12026 C8H9NO4 3-METHYLBENZYL ALCOHOL 3-METHYLBENZYL-ALCOHOL, Acylcarnitine C24:1 MALEIMIDE MALEIMIDE, 4-methylumbelliferone HMDB12163 C03081, D00170 C10H8O3 fluorene FLUORENE, Sedoheptulose C7H14O7 2-INDANONE 2INDANONE, methyl heptadecanoate C18H36O2 3-isochromanone ISOCHROMANONE, 3-hydroxyflavone C01495 C15H10O3 dibenzofuran CPD-926, 5-Cholesten-3-beta-ol=Cholesterol C00187, D00040 C27H46O carbazole CPD-12475, cholesterol d6 C27H46O6-deoxy-D-glucose 6-DEOXY-D-GLUCOSE, cholesterol C27H46O prunin NARINGENIN-7-O-BETA-D-GLUCOSIDE, 4-pyridoxic acid HMDB00017 C0847 C8H9NO4 neohesperidin CPD-7074, Pipecolinic acid HMDB00070 C6H11NO2 chrysin CPD-8184, pipecolic acid HMDB00716 C00408 C6H11NO2 Tricetin CPD-12570, albendazole C01779, D00134 C12H15N3O2S prunetin (4′,5-dihydroxy-7-methoxyisoflavone) CPD-3521, α-D-Glucose 1-phosphate HMDB01586 C6H13O9P fructose Fructose, Glucose-1-phosphate C00103 C6H13O9P phenanthrene CPD-13485, Ethanolamine HMDB00149 C00189, D05074 C2H7NO N-cyclohexylformamide N-CYCLOHEXYLFORMAMIDE, D-galacturonic acid HMDB02545 C6H10O7 beta-Mannosylglycerate 2-O-ALPHA-MANNOSYL-D-GLYCERATE, 3-hydroxybenzaldehyde C03067 C7H6O2 N-ethylglycine CPD-10490, cytidine-5′-monophosphate HMDB00095 C00055 C9H14N3O8P glutaraldehyde CPD-9052, homogentisic acid HMDB00130 C00544 C8H8O4 benzylamine BENZYLAMINE, Digalacturonic acid C02273 C12H18O13 D-Aspartic acid CPD-302, LysoPC(18:0) HMDB10384 C26H54NO7P valine VAL, 3-hydroxybenzoic acid HMDB02466 C0587 C7H6O3 L-asparagine ASN, nonanoic acid methyl ester (Methyl pelargonate) C10H20O2 leucine LEU, 3-isochromanone C07728 C9H8O2 5-hydroxy-L-tryptophan 5-HYDROXY-TRYPTOPHAN, N, N-dimethyl-L-histidine (N(alpha), N(alpha)-dimethyl-L-histidine) C04259 C8H13N3O2 5-methoxypsoralen 5-METHOXYFURANOCOUMARIN, PC(P-16:0/20:4(5Z,8Z,11 Z,14Z)) HMDB11220 C44H8O NO7P D-Fructose 6-phosphate FRUCTOSE-6P, palmitic acid HMDB00220 C00249, D05341 C16H32O2 D-saccharic acid D-GLUCARATE, PC(16:1(9Z)/16:0) HMDB08001 C40H78NO8P Homocystine HOMOCYSTINE, thymidine HMDB00273 C00214 C10H14N2O5 gly-pro (N-Glycyl-L-Proline) CPD-10814, uracil HMDB00300 C00106, D00027 C4H4N2O2 Threonic acid L-THREONATE, benzoin C01408 C14H12O2 erythrose 4-phosphate ERYTHROSE-4P, (−)-Adenosine 3′,5′-cyclic monophosphate HMDB00058 C00575 C10H12N5O6P ribose-5-phosphate RIBOSE-5P, p-cresol HMDB01858 C01468 C7H8O_-D-Glucose 1-phosphate GLC-1-P, 6-hydroxy caproic acid C06103 C6H12O3 D-(+)-Ribonic acid gamma-lactone CPD-13413, 2-hydroxy-2-phenylacetic acid (Mandelic acid) C8H8O3 methionine sulfoxide CPD0-1959, mandelic acid HMDB00703 C01984 C8H8O3 D-galacturonic acid D-GALACTURONATE, methyl palmitoleate C17H32O2 xylitol XYLITOL, tropic acid C9H10O3 D-tagatose TAGATOSE, E-caprolactam C06593 C6H11NO galactinol CPD-458, lactulose C07064, D00352 C12H22O11 2-Monopalmitin CPD66-43, methyl yellow C14H15N3 turanose CPD-13399, GLUTARIC ACID (Glutarate) HMDB00661 C00489 C5H8O4 iminodiacetic acid CPD-10189, methyl octanoate (octanoic acid methyl ester) C9H18O2 Dehydroepiandrosterone 3-BETA-HYDROXYANDROST-5-EN-1 7-ONE, Aminomalonic acid HMDB01147 C0872 C3H5NO4 Prostaglandin E2 CPD-10034, 3-METHYLBENZYL ALCOHOL C07216 C8H10O threonine THR, 5-aminovaleric acid HMDB03355 C00431 C5H11NO2 adenosine-5-monophosphate AMP, coniferyl alcohol C00590 C10H12O3 ascorbate ASCORBATE, p-toluenesulfonic acid HMDB11635 C06677 C7H8O3S L-canavanine CANAVANINE, Threonic acid HMDB00943 C4H8O5 carbamoyl-aspartate CARBAMYUL-L-ASPARTATE, 2-Hydroxyvaleric acid HMDB01863 C5H10O3 Aminooxyacetic acid CARBOXYMETHOXYLAMINE, N-acetyl-ornithine HMDB03357 C00437 C7H14N2O3 Carnitine CARNITINE, 3-ureidopropionate HMDB00026 C02642 C4H8N2O3 cyclic GMP CGMP, 5-METHOXYTRYPTAMINE (methoxytryptamine) HMDB04095 C05659 C11H14N2O cholesterol CHOLESTEROL, DL-p-hydroxyphenyllactic acid HMDB00755 C03672 C9H10O4 4-Cholesten-3-one CPD-323, L-(+) lactic acid HMDB00190 C00186 C3H6O3 (+/−)-Taxifolin Dihydroquercetins, lactic acid C01432, D00111 C3H6O3 cytidine CYTIDINE, Digitoxose C6H12O4 D-Ala-D-Ala D-ALA-D-ALA, kyotorphin HMDB05768 C02993 C15H23N5O4 2_-deoxyadenosine 5_-monophosphate DAMP, shikimic acid HMDB03070 C00493 C7H10O5 allo-inositolÃŠ(myo-inositol)(muco-Inositol) MYO-INOSITOL, hydrocortisone C00735, D00088 C21H30O5 N-methyltryptophan N-METHYLTRYPTOPHAN, lactobionic acid C12H22O12 noradrenaline NOREPINEPHRINE, Mevalonic acid lactone C6H10O3 saccharopine SACCHAROPINE, N-gamma-acetyl-N-2-formyl-5-methoxykynurenamine (N-Acetyl-N-formyl-5-methoxykynurenamine) HMDB04259 C05642 C13H16N2O4 xanthosine XANTHOSINE, N-(2-hydroxyethyl)iminodiacetic acid C6H11NO5 conduritol beta-epoxide CPD-12924, Ethyl cinnamate C06359 C11H12O2 hesperetin CPD-7072, 2-Ketovaleric acid HMDB01865 C06255 C5H8O3 salicin CPD-1142, L-lysine HMDB00182 C00047, D02304 C6H14N2O2 L-ascorbic acid ASCORBATE, succinic acid HMDB00254 C00042 C4H6O4 methyl yellow 4-DIMETHYLAMINOPHENYLAZOBENZENE, heptadecanoic acid HMDB02259 C17H34O2 quinic acid QUINATE, oxaloacetic acid HMDB00223 C00036 C4H4O5 sorbose SORBOSE, 2′-deoxyadenosine 5′-monophosphate C10H14N5O6P (R)-(−)-carvone CPD-1089, putrescine (1,4-Diaminobutane) HMDB01414 C00134,C02896 C4H12N2 3-HYDROXYPYRIDINE CPD-12318, PC(16:0/16:0) HMDB00564 D03585 C40H8O NO8P hexadecane CPD-8509, 4-hydroxyphenylpyruvic acid C01179 C9H8O4 Maleamate MALEAMATE, terephthalic acid HMDB02428 C06337 C8H6O4 Melibiose MELIBIOSE, 3-hydroxycinnamic acid (m-coumaric acid) HMDB01713 C12621 C9H8O3 tetracosane CPD-9764, 3-Methylthiopropylamine (3-(Methylthio)-propylamine) C03354 C4H11NS METHYL PHOSPHATE CPD-10815, 2,3-Dimethylsuccinic acid C6H10O4 3-Cyanoalanine CPD-603, 4-Methylbenzyl alcohol C06757 C8H10O cyclohexane-1,2-diol TRANS-CYCLOHEXANE-12-DIOL, alpha-ketoglutaric acid (2-Ketoglutaric acid) HMDB00208 C00026 C5H6O5 nonanoic acid methyl ester (Methyl pelargonate) CPD-2043, prunetin (4′,5-dihydroxy-7-methoxyisoflavone) C10521 C16H12O5 Dithioerythritol DITHIOERYTHRITOL, Nicotianamine C05324 C12H21N3O6 2,3-DIHYDROXYPYRIDINE CPD-9037, citric acid HMDB00094 C00158, D00037 C6H8O7 (−)-Dihydrocarveol CPD-10027, ferulic acid HMDB00954 C01494 C10H10O4 cysteinylglycine CYS-GLY, Betaine HMDB00043 D07523,C00719 C5H11NO2 D-lyxose LYXOSE, glucoheptonic acid C7H14O8 coprostan-3-one CPD-1082, ACETOPHENONE C07113 C8H8O lactitol CPD0-2460, benzamide HMDB04461 C09815 C7H7NO sophorose CPD-13242, SM(d18:1/14:0) C37H75N2O6P Gentiobiose CPD-3605, β-Sitosterol HMDB00852 C01753 C29H50O D-arabitol CPD-355, gly-pro (N-Glycyl-L-Proline) HMDB00721 C7H12N2O3 Erythrose ERYTHROSE, L-valine HMDB00883 C00183, D00039 C5H11NO2 lactamide CPD-13407, valine C16436 C5H11NO2 D (+)altrose ALTROSE, trehalose-6-phosphate HMDB01124 C00689 C12H23O14P guanidinosuccinic acid CPD-599, DL-2-amino-3-phosphonopropionic acid C3H8NO5P_-D-glucosamine phosphate D-GLUCOSAMINE-6-P, N-carbamyl-L-glutamic acid C6H10N2O5 farnesol CPD-9101, pyruvic acid HMDB00243 C00022 C3H4O3 ribose RIBOSE, 5-hydroxytryptophan HMDB00472 C01017 C11H12N2O3 Sedoheptulose SEDOHEPTULOSE, 5-hydroxy-L-tryptophan D07339, C00643 C11H12N2O3 chlorogenic acid CAFFEOYLQUINATE, uric acid HMDB00289 C00366 C5H4N4O3 N-acetyl-D-mannosamine N-ACETYL-D-MANNOSAMINE, L-cystine HMDB00192 C00491, D03636 C6H12N2O4S2 L-(_)-fucose L-FUCOSE, LysoPC(16:1(9Z)) HMDB10383 C24H48NO7P 1-Methyladenosine 1-METHYLADENOSINE, 4-androsten-7-alpha-ol-3,17-dione (7-Hydroxy-4-androstene-3,17-dione) HMDB06771 C05296 C19H26O3 maltitol CPD-3609, anandamide HMDB04080 C11695 C22H37NO2 loganin LOGANIN, 3-Hydroxynorvaline (DL-ß-Hydroxynorvaline) C5H11NO3 maltotriose MALTOTRIOSE, guaiacol HMDB01398 C01502, D00117,C15572 C7H8O2 Sphingosine SPHINGOSINE, 2-Hydroxyquinoline C06338,C06415 C9H7NO LysoPC(16:0) Lysophosphatidylcholines-16-0, L-4-Hydroxyphenylglycine C12323, D05292 C8H9NO3 LysoPC(18:0) Lysophosphatidylcholines-18-0, N-Acetyl-beta-alanine C01073 C5H9NO3 Betaine BETAINE, 5,6-dimethylbenzimidazole HMDB03701 C03114 C9H10N2 Creatine CREATINE, PE(P-16:0/18:2(9Z,12Z)) HMDB11343 C39H74NO7P Carnitine CARNITINE, 2-Hydroxybiphenyl C02499 C12H10O Acylcarnitine C16:0 CPD-419, adenosine-3′-monophosphate (3′-adenylic acid) HMDB03540 C01367 C10H14N5O7P Acetylcarnitine O-ACETYLCARNITINE, gallic acid HMDB05807 C01424 C7H6O5, urocanic acid HMDB00301 C00785 C6H6N2O2, 15-Keto-prostaglandin F2alpha HMDB04240 C05960 C20H32O5, acetylisatin (N-Acetylisatin) C02172 C10H7NO3, Dodecanol HMDB11626 C02277 C12H26O, xanthine C00385 C5H4N4O2, L-norleucine HMDB01645 C01933 C6H13NO2, 2-phenylacetamide HMDB10715 C02505 C8H9NO, 6-methylprevitamin D C28H46O, resveratrol HMDB03747 C03582 C14H12O3, itaconic acid HMDB02092 C00490 C5H6O4, Bis(2-hydroxypropyl)amine C6H15NO2, pyridoxine HMDB00239 C00314 C8H11NO3, sinapyl alcohol C02325 C11H14O4, Tryptophol HMDB03447 C00955 C1H11NO, dihydrolanosterol HMDB06839 C05109 C30H52O, 20alpha-Hydroxycholesterol (5-CHOLESTEN-3β,20α-DIOL) C05500 C27H46O2, cortisone HMDB02802 D07749, C00762 C21H28O5, thymol HMDB01878 C09908, D01039 C10H14O, 4-acetylbutyric acid C02129 C6H10O3, 21-hydroxypregnenolone C21H32O3, 1,5-Anhydroglucitol HMDB02712 C07326 C6H12O5, 2-Amino-2-norbornanecarboxylic acid C8H13NO2, purine riboside (Purine-9-D-ribofuranoside) C01736,C15586 C10H12N4O4, L-serine HMDB00187 C00065, D00016 C3H7NO3, serine C00716 C3H7NO3, DL-3,4-dihydroxyphenyl glycol HMDB00318 C05576 C8H10O4, DL-Anabasine HMDB04350 C06180 C10H14N2, oxalic acid HMDB02329 C00209 C2H2O4, testosterone HMDB00234 C00535, D00075 C19H28O2, N-acetyl-5-hydroxytryptamine HMDB01238 C00978 C12H14N2O2, vanillin HMDB12308 C00755, D00091 C8H8O3, PE(18:1(9Z)/18:1(9Z)) HMDB09059 C41H78NO8P, tyrosine methyl ester C03404 C10H13NO3, 2-Deoxyuridine HMDB00012 C00526 C9H12N2O5, piperine C03882 C17H19NO3, prostaglandin A2 C05953 C20H30O4, nornicotine ((+/−)-Nornicotine) HMDB01126 C06524 C9H12N2, SM(d18:1/18:1(9Z)) C41H81N2O6P, phosphoric acid (ortho-Phosphoric acid) HMDB02142 C00009, D05467 H3O4P, iminodiacetic acid HMDB11753 C4H7NO4, PC(22:6(4Z,7Z,1 OZ, 13Z,16Z,19Z)/18:1(9Z)) HMDB08729 C48H82NO8P, (−)-perillyl alcohol C02452 C10H16O, phenyl-beta-glucopyranoside (Phenyl beta-D-glucopyranoside) C03097,C11611 C12H16O6, L-menthone C00843 C10H18O, atrazin-2-hydroxy C06552 C8H15N5O, DEHYDROABIETIC ACID C12078 C20H28O2, salicin C13H18O7, erythrose 4-phosphate HMDB01321 C4H9O7P, (+)-6-aminopenicillanic acid C02954 C8H12N2O3S, 4-hydroxycinnamic acid (D-erythro-sphingosine) (p-Coumaric acid) HMDB02035 C00811 C9H8O3, pyridoxal 5′-phosphate HMDB01491 C00018 C8H10NO6P, fluorene C07715 C13H10, cycloleucine C03969 C6H11NO2, 3-(4-hydroxyphenyl)propionic acid HMDB02199 C01744 C9H10O3, mandelonitrile C00561 C8H7NO, 3-hydroxy-3-methylglutaric acid (dicrotalic acid) C03761, D04897 C6H10O5, Aminooxyacetic acid C2H5NO3, PE(P-16:0/20:2(11Z,14Z)) HMDB11349 C41H78NO7P, Allantoic acid HMDB01209 C00499 C4H8N4O4, octanal (OctylAldehyde) HMDB01140 C01545 C8H16O, 5β-ANDROSTAN-17β-OL-3-ONE (dihydrotestosterone) HMDB02961 D07456,C03917 C19H30O2, 2-Amino-3-methyl-1-butanol (Levoglucosan) C5H13NO, guanosine HMDB00133 C0387 C10H13N5O5, (+)-4-cholesten-3-one HMDB00921 C00599 C27H44O, 4-Cholesten-3-one C27H44O, isocitric acid HMDB00193 C00311 C6H8O7, L-pyroglutamic acid (oxoproline) HMDB00267 C01879 C5H7NO3, arginine (L-Arginine) HMDB00517 C00062, D02982 C6H14N4O2, malonic acid HMDB00691 C00383,C02028,C04025 C3H4O4, 2,4-diaminobutyric acid HMDB02362 C4H10N2O2, biuret C06555 C2H5N3O2, 4-isopropylbenzyl alcohol (cuminic alcohol) C06576 C10H14O, 1,2-CYCLOHEXANEDIONE C06105 C6H8O2, orcinol (5-METHYLRESORCINOL) C0727 C7H8O2, adenosine HMDB00050 C00212, D00045 C10H13N5O4, 5,6-dihydrouracil HMDB00076 C00429 C4H6N2O2, 3-hydroxybenzyl alcohol C03351 C7H8O2, CORTICOSTERONE HMDB01547 C02140 C21H30O4, L-proline HMDB00162 C00148, D00035 C5H9NO2, o-Hydroxyhippuric acid HMDB00840 C07588 C9H9NO4, 4-hydroxy-6-methyl-2-pyrone C6H6O3, Cytosine HMDB00630 C00380 C4H5N3O, diethyl oxalpropionate (2,8-Dihydroxyquinoline) C04067 C9H14O5, N-acetyl-L-aspartic acid HMDB00812 C01042 C6H9NO5, DL-dihydrosphingosine C18H39NO2, D-Tyrosine C06420 C9H11NO3, L-tyrosine HMDB00158 C00082, D00022 C9H11NO3, 2-amino-3-(4-hydroxyphenyl)propanoic acid (DL-Tyrosine) C01536 C9H11NO3, N-Acetyl-D-glucosamine HMDB00803 C03878 C8H15NO6, N-acetyl-D-mannosamine C8H15NO6, N-Acetyl-D-galactosamine C01132 C8H15NO6, linoleic acid HMDB00673 C01595 C18H32O2, alpha-Aminoadipic acid HMDB00510 C6H11NO4, 5-cholesten-3-beta-7-alpha-diol (7-alpha-Hydroxycholesterol)(7p-Hydroxycholesterol) C03594 C27H46O2, cyclohexylamine (cyclohexanamine) C00571 C6H13N, 1-Aminocyclopropanecarboxylic acid C01234 C4H7NO2, aniline HMDB03012 C00292 C6H7N, cis-sinapinic acid C11H12O5, 3,5-dimethoxy-4-hydroxycinnamic acid (sinapinic acid) C00482 C11H12O5, glycerol HMDB00131 C00116, D00028 C3H8O3, MALEIMIDE C07272 C4H3NO2, coprostan-3-one C27H46O, 5alpha-cholestan-3-one HMDB00871 C03238 C27H46O, spermine HMDB01256 C00750 C10H26N4, (+)-catechin HMDB02780 C06562 C15H14O6, (−)-epicatechin HMDB01871 C09727 C15H14O6, trans-cyclohexane-1,2-diol C03739 C6H12O2, cyclohexane-1,2-diol C6H12O2, 3-methylcatechol C02923 C7H8O2, (S)-Carnitine HMDB00062 C15025 C7H15NO3, Carnitine C7H15NO3, L-carnitine C00318, D02176 C7H15NO3, Carnitine C00318, D02176 C7H15NO3, D-(+)-Gluconic acid δ-lactone HMDB00150 C00198, D04332 C6H10O6, 3-(1-Pyrazolyl)-L-alanine C01162 C6H9N3O2, PC(20:4(5Z,8Z,11Z,14Z)/18:1 (9Z)) HMDB08433 C46H82NO8P, sucrose-6-phosphate C02591 C12H23O14P, 4-aminophenol HMDB01169 C02372 C6H7NO, trans-4-hydroxy-L-proline HMDB00725 C01157 C5H9NO3, 2-hydroxycinnamic acid (o-coumaric acid) HMDB02641 C01772 C9H8O3, cis-o-coumarinic acid C05838 C9H8O3, 4-hydroxyquinoline C06343 C9H7NO, D-(+)-Fucose C6H12O5, L-(−)-fucose C6H12O5, tetracosane C24H50, lauric acid HMDB00638 C02679 C12H24O2, ribose-5-phosphate HMDB01548 C5H11O8P, ALDOSTERONE C01780 C21H28O5, estriol HMDB00153 C05141, D00185 C18H24O3, PC(20:4(5Z,8Z,11Z,14Z)/16:0) HMDB08429 C44H8O NO8P, nicotinic acid HMDB01488 C00253, D00049 C6H5NO2, palatinose C01742 C12H22O11, 11-beta-prostaglandin-F-2-alpha HMDB10199 C05959 C20H34O5, orotic acid HMDB00226 C00295, D00055 C5H4N2O4, ribose C5H10O5, D-lyxose C5H10O5, D-(+)-Xylose C5H10O5, N-formyl-L-methionine C03145 C6H11NO3S, LysoPE(18:1n9) HMDB11506 C23H46NO7P, sophorose C12H22O11, 3,4-dihydroxycinnamic acid HMDB03501 C01197 C9H8O4, caffeic acid HMDB01964 C01481 C9H8O4, sulfuric acid C00059, D05963 H2O4S, phloroglucinol C02183, D00152 C6H6O3, DL-3-aminoisobutyric acid HMDB03911 C05145 C4H9NO2, methionine sulfoxide HMDB02005 C5H11NO3S, L-methionine sulfoxide C02989 C5H11NO3S, icasonic acid methyl ester (methyl icosanoate) C21H42O2, androsterone C00523 C19H30O2, 3-methyl-2-oxobutanoic acid (2-keto-isovalerate) HMDB00019 C00141 C5H8O3, PE(16:1(9Z)/16:0) HMDB08956 C37H72NO8P, 1-Monopalmitin C19H38O4, hippuric acid HMDB00714 C01586 C9H9NO3, hydroquinone HMDB02434 C00530, D00073 C6H6O2, stearic acid HMDB00827 C01530, D00119 C18H36O2, tryptophan C00806 C11H12N2O2, L-tryptophan HMDB00929 C00078, D00020 C11H12N2O2, PC(18:1(9Z)/16:1(9Z)) HMDB08101 C42H8O NO8P, PC(20:1(11Z)/18:1(9Z)) HMDB08302 C46H88NO8P, 4-Hydroxyquinazoline C8H6N2O, alanine (DL-Alanine) C01401 C3H7NO2, 1-Indanone C01504 C9H8O, cis-1,2-dihydro-1,2-naphthalenediol ((1R,2S)-cis-1,2-dihydro-1,2-naphthalenediol) C04314 C10H10O2, N-Oleoyldopamine C12272 C26H43NO3, SM(d16:1/18:1) C39H77N2O6P, ciliatine HMDB11747 C03557 C2H8NO3P, Homovanillic acid HMDB00118 C05582 C9H10O4, docosanoic acid, methyl ester C23H46O2, 3-(3-hydroxyphenyl)propionic acid HMDB00375 C011457 C9H10O3, melezitose (D-(+)-melezitose) HMDB11730 C08243 C18H32O16, o-cresol HMDB02055 C01542 C7H8O, Butyryl carnitine C11H21NO4, xanthotoxin C01864, D00139 C12H8O4, Cholestan-3beta-ol HMDB00908 C27H48O, cholecalciferol C05443 C27H44O, Lignoceric acid HMDB02003 C08320 C24H48O2, 1,3,5(10)-estratrien-3,6-beta-17-beta-triol C03935 C18H24O3, 5beta-androstane-3,17-dione HMDB03769 C03772 C19H28O2, 5β-ANDROSTAN-3,17-DIONE HMDB00899 C19H28O2, D-Glyceric acid HMDB00139 C00258 C3H6O4, Acetylcarnitine C9H17NO4, N-acetyl-L-glutamic acid C00624 C7H11NO5, alizarin C01474 C14H8O4, 4-hydroxybenzaldehyde HMDB11718 C00633 C7H6O2, 3,4-dihydroxymandelic acid HMDB01866 C05580 C8H8O5, Galactonic acid HMDB00565 C6H12O7, gluconic acid C00770,C00800, C000880,C00257, C000514,C15930 C6H12O7, 2′-Deoxycytidine 5′-triphosphate HMDB00998 C00458 C9H16N3O13P3, L-citrulline HMDB00904 D07706,C00327 C6H13N3O3, tetrahydrocorticosterone C05476 C21H34O4, 1-Methylhydantoin HMDB03646 C02565 C4H6N2O2, 5-beta-cholestan-3-alpha-7-alpha-12-alpha-triol (3,7,12-Trihydroxycoprostane) HMDB01457 C05454 C27H48O3, progesterone HMDB01830 C00410, D00066 C21H30O2, lyxonic acid, 1,4-lactone, 3TMS C14H32O5Si3, hyoscyamine (atropine) C17H23NO3, phytanic acid HMDB00801 C01607 C20H40O2, m-cresol HMDB02048 C01467, D04951 C7H8O, 3-isopropylmalate C04411 C7H12O5, 2-isopropylmalate C7H12O5, tartronic acid C02287 C3H4O5, scopoletin C01752 C10H8O4, leucine C16439 C6H13NO2, L-leucine HMDB00687 C00123, D00030 C6H13NO2, guanosine-5′-monophosphate HMDB01397 C00144 C10H14N5O8P, PC(18:1(9Z)/16:0) HMDB08100 C42H82NO8P, 3-Indolepyruvic acid C00331 C11H9NO3, abietic acid C06087 C20H30O2, PC(18:2(9Z,12Z)/18:1(9Z)) HMDB08137 C44H82NO8P, chrysin C10028 C15H10O4, turanose HMDB11740 C12H22O11, SM(d18:1/16:0) C39H79N2O6P, thymine HMDB00262 C00178 C5H6N2O2, L-glutathione HMDB00125 C00051,C02471, D00014 C10H17N3O6S, Monoolein (1-Oleoyl-rac-glycerol) C21H40O4, adrenosterone HMDB06772 C05285 C19H24O3, palmitoleic acid C08362 C16H30O2, 3-indoleacetic acid C00954 C10H9NO2, palatinitol C12H24O11, norepinephrine (noradrenaline) HMDB00216 C00547, D00076 C8H11NO3, noradrenaline C8H11NO3, D-Erythronic acid gamma-Lactone HMDB00349 C4H6O4, 6-deoxy-D-glucose C08352 C6H12O5, picolinic acid HMDB02243 C10164 C6H5NO2, 1-Hydroxy-2-naphthoic acid C03203 C11H8O3, 4-hydroxybutyrate HMDB00710 C00989,C01991 C4H8O3, dehydroshikimic acid C7H8O5, 2-FUROIC ACID HMDB00617 C01546 C5H4O3, salicylaldehyde C06202 C7H6O2, norvaline C01826 C5H11NO2, PC(18:1(9Z)/18:1(9Z)) C44H84NO8P, D-panthenol C05944, D00193 C9H19NO4, oxamic acid C01444 C2H3NO3, Hydantoin, 5-(4-hydroxybutyl) C7H12N2O3, O-phosphocolamine (O-phosphorylethanolamine) HMDB00224 C00346 C2H8NO4P, Acylcarnitine C20:2, N-cyclohexylformamide C11519 C7H13NO, 2-HYDROXYESTRONE HMDB00343 C05298 C18H22O3, 1,3,5(10)-ESTRATRIEN-2,3-DIOL-17-ONE C18H22O3, 4-Hydroxy-4-methyl-2-pentanone C6H12O2, 2-Amino-3-methoxybenzoic acid C05831 C8H9NO3, lactamide C3H7NO2, glutaraldehyde C12518, D01120 C5H8O2, L-gulonic acid γ-lactone HMDB03466 C01040 C6H10O6, myristic acid HMDB00806 C06424 C14H28O2, Acylcarnitine C12:1, dibenzofuran C07729 C12H8O, trans,trans-muconic acid HMDB02349 C6H6O4, 2-oxobutyrate HMDB00005 C00109 C4H6O3, L-ascorbic acid C6H8O6, ascorbate C6H8O6, xanthosine C10H12N4O6, 2-hydroxypyridine C02502 C5H5NO, Beta-alanine HMDB00056 D07561,C00099 C3H7NO2, adrenaline ((−)-Epinephrine) HMDB00068 C00788, D00095 C9H13NO3, Adenosine 5′-monophosphate HMDB00045 C00020, D02769 C10H14N5O7P, adenosine-5-monophosphate C10H14N5O7P, 2-aminoethanethiol HMDB02991 C01678, D03634 C2H7NS, naphthalene C00829 C10H8, Glucosaminic acid C6H13NO6, inosine HMDB00195 C00294, D00054 C10H12N4O5, cytidine C9H13N3O5, PE(18:1(9Z)/16:1(9Z)) HMDB09056 C39H74NO8P, 6-methylmercaptopurine C16614 C6H6N4S, N-(3-aminopropyl)-morpholine C7H16N2O, succinate semialdehyde HMDB01259 C00232 C4H6O3, PC(16:0/14:0) HMDB07965 C38H76NO8P, carbazole C08060 C12H9N, L-homoserine HMDB00719 C00263 C4H9NO3, behenic acid HMDB00944 C08281 C22H44O2, (R)-(−)-carvone C10H14O, 2-amino-1-phenylethanol HMDB01065 C02735 C8H11NO, 2-INDANONE C07727 C9H8O, D-threitol HMDB04136 C16884 C4H10O4, alpha-Ecdysone C00477 C27H44O6, 2-Monoolein (2-oleoylglycerol) HMDB11537 C211-14004, dodecanoic acid methyl ester (methyl dodecanoate) C13H26O2, 8-aminocaprylic acid C8H17NO2, PE(P-18:0/20:4) HMDB05779 C43H78NO7P, N-carbobenzyloxy-L-leucine C04335 C14H19NO4, O-phospho-L-threonine HMDB11185 C12147 C4H10NO6P, O-phosphonothreonine C4H10NO6P, PC(20:3(8Z,11Z,14Z)/18:0) HMDB08399 C46H86NO8P, phenaceturic acid HMDB00821 C05598 C10H11NO3, PE(P-16:0/20:4(5Z,8Z,11Z,14Z)) HMDB11352 C41H74NO7P, 2-amino-2-methyl-1,3-propanediol C11260 C4H11NO2, lumazine C03212 C6H4N4O2, 4-acetamidobutyric acid HMDB03681 C02946 C6H11NO3, 1-Kestose HMDB11729 C03661 C18H32O16, Monostearin (1-Stearoyl-rac-glycerol) D01947 C21H42O4, 2-Keto-L-gulonic acid C6H10O7, galactinol HMDB05826 C12H22O11, D-glucose-6-phosphate C03251 C6H13O9P, PE(20:4(5Z,8Z,11Z,14Z)/18:1(9Z)) HMDB09389 C43H76NO8P, phloretin HMDB03306 C00774 C15H14O5, flavone HMDB03075 C10043,C15608 C15H10O2, L-dithiothreitol C00265 C4H10O2S2, Dithioerythritol C4H10O2S2, 1,4-dithioerythritol C00950 C4H10O2S2, hexacosanoic acid methyl ester (METHYL HEXACOSANOATE) C27H54O2, arachidic acid HMDB02212 C06425 C20H40O2, dihydrocoumarin C02274 C9H8O2, 1,4-Dihydroxy-2-naphthoic acid C03657 C11H8O4, 3-phenyllactic acid HMDB00779,HMDB00563 C9H10O3, Epiestradiol HMDB00429 D07121,C02537 C18H24O2, 1,3,5(10)-ESTRATRIEN-3,17a-DIOL (estradiol) HMDB00151 C00951, D00105 C18H24O2, maltitol C12H24O11, lactitol C12H24O11, Cellobiotol C12H24O11, 4-quinolinecarboxylic acid C06414 C10H7NO2, 2-deoxy-D-glucose (2-Deoxy-D-galactose) C6H12O5, hydroxyurea C07044, D00341 CH4N2O2, guanidinosuccinic acid C5H9N3O4, flavin adenine dinucleotide HMDB01248 C00016, D00005 C27H33N9O15P2, LysoPC(14:0) HMDB1 0379 C22H46NO7P, fumaric acid HMDB00134 C00122, D02308 C4H4O4, maleic acid HMDB00176 C01384 C4H4O4, acetyl-L-serine (O-acetylserine) C5H9NO4, cinnamic acid HMDB00567 C00423,C10438 C9H8O2, benzylamine C15562 C7H9N, Cortexolone (Reichstein's Substances) C21H30O4, 3-hydroxybutyric acid HMDB00357 C4H8O3, (S)-3-Hydroxybutyric acid HMDB00442 C03197 C4H8O3, 2-METHOXYESTRONE [1,3,5(10)-ESTRATRIEN-2,3-DIOL-17-ONE2-METHYLETHER] HMDB00010 C05299 C19H24O3, glutamic acid C00302, D04341 C5H9NO4, L-Glutamic acid HMDB00148 C00025, D00007 C5H9NO4, Methoxamedrine C07513 C11H17NO3, 3,4-dimethoxybenzaldehyde C02201 C9H10O3, 3-hydroxyanthranilic acid HMDB01476 C00632 C7H7NO3, vanillic acid (4-hydroxy-3-methoxybenzoic acid) HMDB00484 C06672 C8H8O4, pimelic acid HMDB00857 C02656 C7H12O4, PHENYLACETIC ACID HMDB00209 C07086 C8H8O2, adipic acid HMDB00448 C06104 C6H10O4, 3-Methylamino-1,2-propanediol C4H11NO2, benzoic acid HMDB01870 C00180,C00539, D00038 C7H6O2, pentadecanoic acid HMDB00826 C16537 C15H30O2, pyrogallol C01108 C6H6O3, galactose C00124 C6H12O6, pyrrole-2-carboxylic acid HMDB04230 C05942 C5H5NO2, malonamide C3H6N2O2, (−)-Dihydrocarveol C10H18O, 4-androsten-11-beta-ol-3,17-dione HMDB06773 C05284 C19H26O3, 3-(3,4-Dihydroxyphenyl)-L-alanine [L-DOPA] HMDB00181 C00355, D00059 C9H11NO4, L-3,4-Dihydroxyphenylalanine HMDB00609 C9H11NO4, citral C01499 C10H16O, Linoleic acid methyl ester C19H34O2, 5,7-dihydroxy-3-(4-methoxyphenyl)chromen-4-one (Biochanin A=5,7-Dihydroxy-4′-Methoxyisoflavone) HMDB02338 C00814 C16H12O5, 5′-deoxy-5′-(methylthio)adenosine (5′-methylthioadenosine) HMDB01173 C00170 C11H15N5O3S, benzyl alcohol HMDB03119 C00556,C03485, D00077 C7H8O, PE(P-16:0/22:6) HMDB05780 C43H74NO7P, N-methylanthranilic acid C03005 C8H9NO2, Sphingosine C00319 C18H37NO2, caprylic acid HMDB00482 C06423, D05220 C8H16O2, N-acetyl-L-leucine HMDB11756 C02710 C8H15NO3, gentisic acid HMDB00152 C0628 C7H6O4, paraoxon ethyl C06606 C10H14NO6P, 3,6-Anhydro-D-galactose C06474 C6H10O5, Prostaglandin E2 C20H32O5, 1,3-diaminopropane HMDB00002 C00986 C3H10N2, citramalic acid C02612 C5H8O5, 3-indolelactic acid (Indolelactate) HMDB00671 C02043 C11H11N3, 4-Androsten-19-ol-3,17-dione HMDB03955 C05290 C19H26O3, maltotriitol C18H34O16, alpha-santonin C02206, D00154 C15H18O3, 3-METHYLGLUTARIC ACID HMDB00752 C6H10O4, 4-nitrocatechol HMDB02916 C02235 C6H5NO4, Nw-acetylhistamine (N-omega-Acetylhistamine) C05135 C7H11N3O, N-Methyl-L-glutamic acid C01046 C6H11NO4, acetol HMDB06961 C05235 C3H6O2, Cerotinic acid HMDB02356 C26H52O2, hydrocinnamic acid HMDB00764 C05629 C9H10O2, epigallocatechin C12136 C15H14O7, 5-alpha-pregnan-3,20-dione (5-alpha-Dihydroprogesterone; 5a-PREGNAN-3,20-DIONE) HMDB03759 C03681 C21H32O2, phthalic acid HMDB02107 C01606 C8H6O4, 2-ketocaproic acid HMDB01864 C00902 C6H10O3, taurine HMDB00251 C00245, D00047 C2H7NO3S, Acylcarnitine C16:0 C23H45NO4, pyrophosphate C00013 H4O7P2, D-Fructose 1,6-bisphosphate C6H14O12P2, 4-hydroxyphenylacetic acid HMDB00020 C00642 C8H8O3, urea HMDB00294 C00086, D00023 CH4N2O, tetracosanoic acid methyl ester (Methyl tetracosanoate) C25H50O2, L-Cysteine HMDB00574 C00097, D00026 C3H7NO2S, L-cysteine C00736 C3H7NO2S, 2-piperidone (5-aminovaleric acid lactam) HMDB11749 C5H9NO, Glutaconic acid HMDB00620 C02214 C5H6O4, L-cysteic acid C00506 C3H7NO5S, 4-nitrophenyl phosphate HMDB01300 C03360 C6H6NO6P, PC(22:6(4Z,7Z,10Z,13Z,16Z,19Z)/16:0) HMDB08725 C46H8O NO8P, LysoPC(18:1(9Z)) HMDB02815 C26H52NO7P, biotin HMDB00030 C00120, D00029 C10H16N2O3S, 2-(4-hydroxyphenyl)ethanol HMDB04284 C06044 C8H10O2, catechol HMDB00957 C00090,C01785,C15571 C6H6O2, 5-Dihydrocortisone [S5a-PREGNAN-17,21-DIOL-3,11,20-TRIONE] HMDB06758 C05469 C21H30O5, N,N-dimethylarginine HMDB01539 C03626 C8H18N4O2, L-kynurenine HMDB00684 C00328 C10H12N2O3, salicylic acid (o-Hydroxybenzoic acid) HMDB01895 C00805, D00097 C7H6O3, 3-Hexenedioic acid HMDB00393 C6H8O4, 4-nitroquinoline N-oxide C03474 C9H6N2O3, farnesal C03461 C15H24O, DL-4-hydroxymandelic acid HMDB00822 C11527 C8H8O4, 1-INDANOL C01710 C9H10O, oxamide C2H4N2O2, 10-Hydroxydecanoic acid C02774 C10H20O3, 2′-deoxyguanosine HMDB00085 C00330 C10H13N5O4, 2,3-dihydroxybiphenyl (3-phenylcatechol) C02526 C12H10O2, 9-fluorenone C06712 C13H8O, phosphomycin C3H7O4P, CHOLESTAN-3β,5α,6β-TRIOL (Cholestane-3,5,6-triol, (3.beta.,5.alpha.,6.beta.)-) HMDB03990 C05425 C27H48O3, phenanthrene C11422 C14H10, N-ethylglycine C11735 C4H9NO2, d-Glucoheptose C7H14O7, 3,4-dihydroxybenzoic acid HMDB01856 C00230 C7H6O4, (+/−)-Synephrine C9H13NO2, synephrine HMDB04826 D07148,C04548 C9H13NO2, decanoic acid methyl ester (methyl decanoate) C11H22O2, Erythrose C4H8O4, citric acid HMDB00094 C00158, D00037 C6H8O7, Fructose 2,6-biphosphate HMDB01047 C00665 C6H14O12P2, squalene C00751 C30H50, arachidonic acid HMDB01043 C00219 C20H32O2, tocopherol acetate D01735 C31H52O3, tetradecanoic acid methyl ester (Methyl myristate) C15H30O2, 4-methylcatechol HMDB00873 C06730 C7H8O2, nicotinoylglycine HMDB03269 C05380 C8H8N2O3, halostachine C9H13NO, 3,4-Dihydroxypyridine C02932,C03927 C5H5NO2, saccharopine C11H20N2O6, L-Saccharopine HMDB00279 C00449 C11H20N2O6, L-glutamine HMDB00641 C00064, D00015 C5H10N2O3, Allylmalonic acid C6H8O4, 4-hydroxy-3-methoxybenzyl alcohol 06317 C8H10O3, p-anisic acid HMDB01101 C02519 C8H8O3, conduritol beta-epoxide C6H10O5, methylmalonic acid HMDB00202 C02170 C4H6O4, 22-Ketocholesterol C27H44O2, 7,8-dimethylalloxazine C01727 C12H10N4O2, 4-Hydroxymethyl-3-methoxyphenoxyacetic acid C10H12O5, octacosanoic acid methyl ester C29H58O2, aniline-o-sulfonic acid C06333 C6H7NO3S, 2-mercaptoethanesulfonic acid HMDB03745 C03576 C2H6O3S2, indole-3-acetamide C02693 C10H10N2O, flavanone C00766 C15H12O2, cyclic GMP C10H12N5O7P, Guanosine 3′,5′-cyclic monophosphate HMDB01314 C00942 C10H12N5O7P, oleic acid HMDB00207 C00712, D02315 C18H34O2, Elaidic acid HMDB00573 C01712 C18H34O2, daidzein HMDB03312 C10208 C15H10O4, butyraldehyde HMDB03543 C01412 C4H8O, L-homocystine C01817 C8H16N2O4S2, Homocystine HMDB00676 C8H16N2O4S2, cysteinylglycine C5H10N2O3S, biphenyl C06588 C12H10, PE(P-16:0/18:1 (9Z)) HMDB11342 C39H76NO7P, 2,4-dihydroxypyrimidine-5-carboxylic acid (uracil-5-carboxylic acid) C03030 C5H4N2O4.

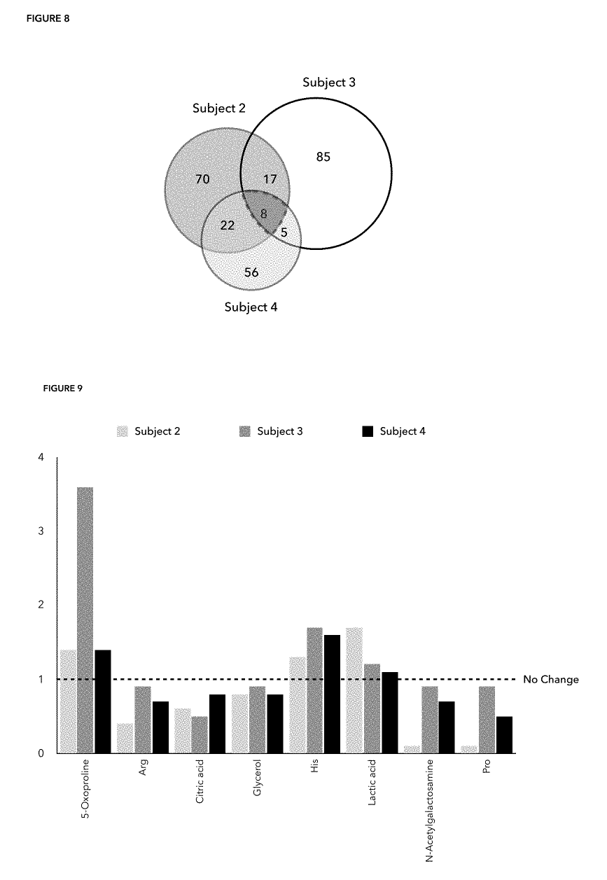

FIG. 8 illustrates a Venn Diagram of the number of biomarker candidates that significantly change 10 min post-consumption of cannabis in subjects 2, 3 and 4.

FIG. 9 illustrates the levels of the biomarker candidates exhibiting similar patterns 10 min post-consumption of cannabis in subjects 2, 3 and 4.

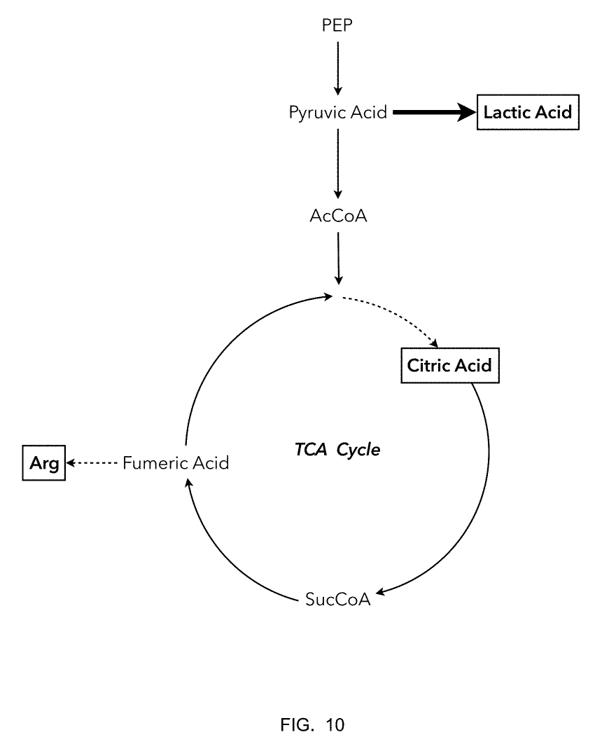

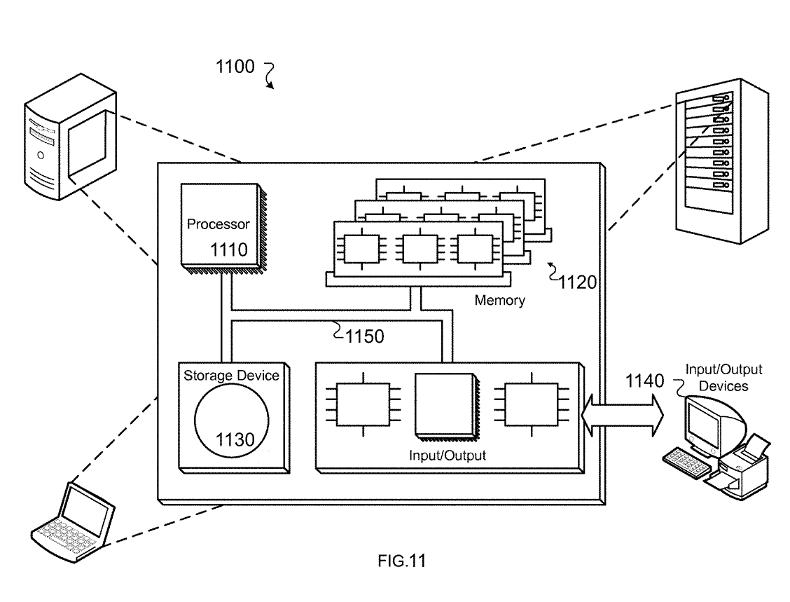

FIG. 10 illustrates the TCA metabolic pathway. Framed metabolites represent biomarker candidates identified in FIG. 9. Wide arrow indicates increased levels of biomarker and dashed arrow indicates decreased levels of biomarker.

FIG. 11 illustrates a system environment diagram, in which various embodiments may be implemented. A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, may be based on general or special purpose microprocessors or both, or any other kind of central processing unit including graphics processing units. Generally, a central processing unit will receive instructions and data from a read only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, embodiments of the subject matter described in this specification may be implemented on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input. In addition, a computer may interact with a user by sending documents to and receiving documents from a device that is used by the user; for example, by sending web pages to a web browser on a user's client device in response to requests received from the web browser.

Embodiments of the subject matter described in this specification may be implemented in a computing system that includes a back end component, e.g., as a data server, or that includes a middleware component, e.g., an application server, or that includes a front end component, e.g., a client computer having a graphical user interface or a Web browser through which a user may interact with an implementation of the subject matter described in this specification, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

An example of one such type of computer is shown in FIG. 11, which shows a schematic diagram of a generic computer system 1100. The system 1100 can be used for the operations described in association with any of the computer-implement methods described previously, according to one implementation. The system 1100 includes a processor 1110, a memory 1120, a storage device 1130, and an input/output device 1140. Each of the components 1110, 1120, 1130, and 1140 are interconnected using a system bus 1150. The processor 1110 is capable of processing instructions for execution within the system 1100. In one implementation, the processor 1110 is a single-threaded processor. In another implementation, the processor 1110 is a multi-threaded processor. The processor 1110 is capable of processing instructions stored in the memory 1120 or on the storage device 1130 to display graphical information for a user interface on the input/output device 1140.

The memory 1120 stores information within the system 1100. In one implementation, the memory 1120 is a computer-readable medium. In one implementation, the memory 1120 is a volatile memory unit. In another implementation, the memory 1120 is a non-volatile memory unit.

The storage device 1130 is capable of providing mass storage for the system 1100. In one implementation, the storage device 1130 is a computer-readable medium. In various different implementations, the storage device 1130 may be a floppy disk device, a hard disk device, an optical disk device, or a tape device.

The input/output device 1140 provides input/output operations for the system 1100. In one implementation, the input/output device 1140 includes a keyboard and/or pointing device. In another implementation, the input/output device 1140 includes a display unit for displaying graphical user interfaces.

### EXAMPLES

The invention will be further explained by the following illustrative examples that are intended to be non-limiting.

### Example 1: Screening and Identification of Cannabis Consumption-Associated Metabolic Biomarker Candidates in Different Subjects

This example describes the determination of metabolites that vary in level pre- and post-cannabis consumption. Saliva samples collected from subjects are described in Table 1. Subject 1 is a non-cannabis consumer that serves as control.

TABLE 1
Composition of subjects by demographics and and medical cannabis treatment

| | | Subject and treatment | | | |
|---|---|---|---|---|---|
| | | Subject 1 | Subject 2 | Subject 3 | Subject 4 |
| Gender | | Male | Male | Male | Female |
| Racial and Ethnic Categories | | White | White | White | White |
| Age Group | | 51-65 | 36-50 | 36-50 | 51-65 |
| Primary Use of | | Control, | Pain | Anxiety | Pain, Insomnia |
| Medical Cannabis | | Not using cannabis | | | |
| Method of Consumption | | NA | Vape | Vape | Edible |
| Product | Type | NA | Flower | Flower | Dark chocolate |
| | THC (%) | NA | 23.5 | 9.8 | 3 |
| | CBD (%) | NA | 0.06 | 7.3 | 3 |
| Previous consumption (Hrs) | | 0 | 4 | 24 | >72 |
| Sampling time | Pre (min) | NA | NA | 2 | 60 |
| | Post (min) | NA | 10 | 10 | 10 |
| | Post (min) | NA | NA | 60 | 60 |
| | Post (min) | NA | NA | 120 | 500 |

Pre-consumption refers to a time point prior to cannabis consumption by the cannabis consumption by the method described in Table 1.

To measure cannabis consumption accurately subjects described in Table 1 avoided a major meal 60 min pre-consumption, rinsed mouth immediately after consumption with water and waited 10 min prior to saliva collection. Saliva samples (0.5 ml-1 ml) were collected using the Saliva Passive Drool Collection Kit (Salimetrics, LLC, Carlsbad, Calif.) and immediately frozen on dry ice.

Prior to metabolome testing, frozen saliva was thawed and dissolved at room temperature. Prior to the metabolome analyses, each saliva sample (50 μL) was mixed with 20 μL of Milli-Q (Merck Millipore, Billerica, Mass., USA) containing internal standards and 20 mM each of methionine sulfone, D-camphor-10-sulfonic acid (Wako Pure Chemical Industries, Ltd. Osaka, Japan), 2-(n-morpholino) ethanesulfonic acid (Dojindo Molecular Technologies, Inc., Kumamoto, Japan), 3-aminopyrrolidine (Sigma-Aldrich Japan K.K., Tokyo, Japan), and trimesate (Wako Pure Chemical Industries, Ltd.) and 30 μL of Milli-Q water. The mixture was then filtered through 5-kDa cut-off filter (ULTRAFREE-MC-PLHCC, Human Metabolome Technologies, Yamagata, Japan) to remove macromolecules.

The compounds were measured in the Cation and Anion modes of CE-TOFMS (Agilent Technologies, Santa Clara, Calif.) based metabolome as described by Soga et al., (2003).

Peaks detected in CE-TOFMS analysis were extracted using automatic integration software (MasterHands ver. 2.17.1.11 developed at Keio University) in order to obtain peak information including m/z, migration time (MT), and peak area. The peak area was then converted to relative peak area by the following equation described below. The peak detection limit was determined based on signal-noise ratio; S/N=3.

$$Relative Peak Area = \frac{Metabolite Peak Area}{Internal Standard Peak Area \times Sample Amount}$$

Putative metabolites were then assigned from HMT (Human Metabolome Technologies Inc Tokyo, Japan) standard library and Known-Unknown peak library on the basis of m/z and MT. The tolerance was ±0.5 min in MT and ±10 ppm* in m/z. If several peaks were assigned the same candidate, the candidate was given the branch number.

$${ }^{*} Mass error ( ppm ) = \frac{Measured Value - Theoretical Value}{Measured Value} \times {10}^{6}$$

Hierarchical cluster analysis (HCA) and principal component analysis (PCA) were performed by statistical analysis software (developed at HMT).

The profile of peaks with putative metabolites are represented on metabolic pathway maps using VANTED (Visualization and Analysis of Networks containing Experimental Data) software. The pathway map was prepared based on the metabolic pathways that are known to exist in human cells according to the information in KEGG database (http://www.genome.jp/kegg/).

TABLE 2
Summary of sample analysis

| | | Number of | Compounds that | | | |
|---|---|---|---|---|---|---|
| | | Scanned | significantly change* | | | |
| | Analysis | molecules/ | Primary | Basic | Proline | |
| | Type | sample | Metabolite | peptides | peptides | Total |
| Subject 2 | Basic | 900 | 70 | N.A | N.A | 70 |
| | CE/MS | | | | | |
| Subject 3 | High | 4000 | 85 | 20 | 19 | 124 |
| | Resolution | | | | | |
| | CE/MS | | | | | |
| Subject 4 | Basic | 900 | 56 | N.A | N.A | 56 |
| | CE/MS | | | | | |

*At least 50% increase or decrease in metabolite ratio of Post-consumption/Pre-consumption

Listed in Table 2 is the summary of scanned metabolites exhibiting significant changes (above 50% increase or decrease) post-consumption.

$$Fold change = \frac{Relative Peak Area at the indicated time post \text{-} consumption}{Relative Peak Area Pre \text{-} Consumption}$$

The Basic CE/MS scan covered metabolites related to central carbon metabolism, protein and DNA turnover and other primary metabolism that were detected in the saliva from the subjects described in Table 1. The High-Resolution CE/MS included the targets described above, peptides and unknown metabolites. To determine changes in subject 2, the values of Subject 3 pre-consumption were set as baseline for subject 2.

Table 3 describes 70 known and unknown metabolites detected in subject 2 10 min post-consumption. A “>10” indicates appearance of a metabolite that was not detected pre-consumption and a “<10” indicates a metabolite that disappears post-consumption. Pathway Index describes the categories on the basis of metabolic pathways (KEGG) and biological functions (HMDB) of candidate compounds.

TABLE 3
Compounds that significantly change post-consumption in Subject 2

| | | Fold change | |
|---|---|---|---|
| # | Candidates | after 10 min | Pathway Index |
| 1 | 2-Hydroxy-4-methylvaleric acid | >10 | Organic Acids |
| 2 | 2-Hydroxybutyric acid | >10 | Lipid and amino acid metabolism |
| 3 | 2-Hydroxyvaleric acid | >10 | Organic Acids |
| 4 | 4-Methyl-2-oxovaleric acid | >10 | BCAA & aromatic amino acids |
| 5 | Betaine | >10 | Lipid and amino acid metabolism |
| 6 | GABA | >10 | Urea cycle relating metaboloism |
| 7 | Hydroxyproline | >10 | Urea cycle relating metaboloism |
| 8 | Nicotine | >10 | Metabolism of coenzymes |
| 9 | Octanoic acid | >10 | Lipid and amino acid metabolism |
| 10 | Paraxanthine | >10 | Lipid and amino acid metabolism |
| 11 | Ribulose 5-phosphate | >10 | Central carbon metabolism |
| 12 | Theobromine | >10 | Alkaloid |
| 13 | Xanthine | >10 | Nucleotide metabolism |
| 14 | XC0016 | >10 | Umkown |
| 15 | XC0061 | >10 | Umkown |
| 16 | Histamine | 7.6 | Urea cycle relating metaboloism |
| 17 | Caffeine | 6.7 | Stimulants |
| 18 | Hypoxanthine | 6.3 | Nucleotide metabolism |
| 19 | Spermine | 6.0 | Urea cycle relating metaboloism |
| 20 | Nicotinic acid | 5.8 | Metabolism of coenzymes |
| 21 | XC0001 | 5.8 | Umkown |
| 22 | Choline | 5.1 | Lipid and amino acid metabolism |
| 23 | Succinic acid | 4.1 | Central carbon metabolism/Urea cycle relating metaboloism |
| 24 | Creatinine | 4.1 | Urea cycle relating metaboloism |
| 25 | Inosine | 3.5 | Nucleotide metabolism |
| 26 | Uridine | 3.3 | Nucleotide metabolism |
| 27 | Taurine | 3.1 | Lipid and amino acid metabolism |
| 28 | Guanosine | 3.0 | Nucleotide metabolism |
| 29 | Cytidine | 3.0 | Nucleotide metabolism |
| 30 | O-Acetylcarnitine | 2.9 | Lipid and amino acid metabolism |
| 31 | Ethanolamine phosphate | 2.5 | Sphingolipids metabolism |
| 32 | Guanine | 2.2 | Nucleotide metabolism |
| 33 | Glycerol 3-phosphate | 2.2 | Central carbon metabolism/Lipid and amino acid metabolism |
| 34 | N⁸-Acetylspermidine | 2.1 | Organic compounds |
| 35 | Adenine | 2.1 | Nucleotide metabolism |
| 36 | Ethanolamine | 2.0 | Depression bomarker |
| 37 | Cadaverine | 1.9 | Poly amine, Lysine degradation |
| 38 | Dyphylline | 1.7 | Caffeine metabolism |
| 39 | Uric acid | 1.7 | Nucleotide metabolism |
| 40 | Lactic acid | 1.7 | Central carbon metabolism/Urea cycle relating metaboloism |
| 41 | Phe | 1.6 | BCAA & aromatic amino acids |
| 42 | 3-Hydroxybutyric acid | 1.6 | Central carbon metabolism/Lipid and amino acid metabolism |
| 43 | Urea | 1.6 | Urea cycle relating metaboloism |
| 44 | Carnitine | 1.5 | Lipid and amino acid metabolism |
| 45 | Sarcosine | 1.5 | Lipid and amino acid metabolism |
| 46 | Spermidine | 0.5 | Urea cycle relating metaboloism |
| 47 | Trimethylamine | 0.5 | Methane metabolism |
| 48 | 2-Hydroxyglutaric acid | 0.5 | Butanoate metabolism |
| 49 | Ala | 0.5 | Central carbon metabolism/Urea cycle relating metaboloism/BCAA & aromatic |
| | | | amino acids |
| 50 | 1-Methyl-4-imidazoleacetic acid | 0.5 | Urea cycle relating metaboloism |
| 51 | γ-Butyrobetaine | 0.5 | Lipid and amino acid metabolism |
| 52 | N-Acetylneuraminic acid | 0.5 | Urea cycle relating metaboloism |
| S3 | Ser | 0.4 | Lipid and amino acid metabolism |
| 54 | Gln | 0.4 | Urea cycle relating metabolism |
| 55 | Arg | 0.4 | Central carbon metabolism/Urea cycle relating metaboloism |
| 56 | Trp | 0.4 | BCAA & aromatic amino acids |
| 57 | Urocanic acid | 0.4 | Urea cycle relating metaboloism |
| 58 | Propionic acid | 0.4 | Lipid and amino acid metabolism/BCAA & aromatic amino acids |
| 59 | N-Acetylputrescine | 0.3 | Urea cycle relating metaboloism |
| 60 | Lys | 0.3 | Lipid and amino acid metabolism |
| 61 | Putrescine | 0.3 | Urea cycle relating metaboloism |
| 62 | Hexanoic acid | 0.2 | Methane metabolism |
| 63 | Citrulline | 0.2 | Organic Acids |
| 64 | Gly | 0.2 | Urea cycle relating metaboloism/Lipid and amino acid metabolism |
| 65 | Ornithine | 0.1 | Urea cycle relating metaboloism |
| 66 | 5-Aminovaleric acid | 0.1 | Bacterial Transformation |
| 67 | N-Acetylgalactosamine | 0.1 | Galactose metabolis |
| 68 | Pro | 0.1 | Urea cycle relating metaboloism |
| 69 | Isobutyric acid | 0.1 | Degradation of aromatic compounds |
| | Butyric acid | | |
| 70 | 3-(4-Hydroxyphenyl)propionic acid | 0.1 | Lipid and amino acid metabolism/BCAA & aromatic amino acids |

Table 4 contains a list of the biochemical pathways that show differences post-consumption (verses pre-consumption) in subject 2. The number of biomarker candidates per biochemical pathway is indicated.

TABLE 4
Number of biomarker candidates showing differences post consumption
per biochemical pathway in subject 2

| | Number |
|---|---|
| Pathway Index | Candidates |
| Urea cycle relating metabolism | 15 |
| Lipid and amino acid metabolism | 12 |
| Nucleotide metabolism | 9 |
| Central carbon metabolism/Lipid and amino acid | 5 |
| metabolism | |
| BCAA & aromatic amino acids | 3 |
| Organic Acids | 3 |
| Umkown | 3 |
| Lipid and amino acid metabolism/BCAA & | 2 |
| aromatic amino acids | |
| Metabolism of coenzymes | 2 |
| Methane metabolism | 2 |
| Alkaloid | 1 |
| Bacterial Transformation | 1 |
| Butanoate metabolism | 1 |
| Caffeine metabolism | 1 |
| Central carbon metabolism | 1 |
| Central carbon metabolism/Urea cycle relating | 1 |
| metaboloism/BCAA & aromatic amino acids | |
| Degradation of aromatic compounds | 1 |
| Depression bomarker | 1 |
| Galactose metabolis | 1 |
| Organic compounds | 1 |
| Poly amine, Lysine degradation | 1 |
| Sphingolipids metabolism | 1 |
| Stimulants | 1 |
| Urea cycle relating metaboloism/Lipid and amino | 1 |
| acid metabolism | |
| Total | 70 |

Table 5 describes 85 known and unknown metabolites detected in subject 3 10 min and 60 min post-consumption. A “>10” indicates appearance of metabolite that was not detected pre-consumption and a “<10” indicates metabolite that disappears post-consumption. A “>70” indicates an increase of 7-fold over the value of the

TABLE 5
Compounds that significantly change post-consumption in subject 3

| | | Fold of change | Fold of change | |
|---|---|---|---|---|
| # | Compound | after 10 min | after 60 min | Pathway Index |
| 1 | C26H38O3 (steroidal) | >10 | >70 | Glucose Metabolism |
| 2 | 2-Hydroxy-4-methylvaleric acid | 0.0 | >10 | Organic Acids |
| 3 | C6H12O6 | 0.0 | >10 | Polyols |
| 4 | Caffeine | <0.1 | <0.1 | Stimulants |
| 5 | Succinic acid | 1.0 | 6.4 | Central carbon metabolism/Urea cycle relating metaboloism |
| 6 | Histamine | 2.1 | 6.0 | Urea cycle relating metaboloism |
| 7 | Dihydroxymethylvalerolactone | 1.1 | 4.9 | Organic Acids |
| 8 | Butyric acid | 1.0 | 4.6 | Organic Acids |
| 9 | 6-Aminooctahydroindolizin-1-yl acetate | 1.3 | 4.2 | exocrine function, especially salivation |
| 10 | Spermine | 0.0 | 4.1 | Urea cycle relating metaboloism |
| 11 | Leu | 1.4 | 3.7 | BCAA & aromatic amino acids |
| 12 | Val | 1.7 | 3.2 | BCAA & aromatic amino acids |
| 13 | Ethanolamine phosphate | 0.4 | 3.2 | Sphingolipids metabolism |
| 14 | 5-Aminovaleric acid | 1.4 | 3.1 | Bacterial Transformation |
| 15 | Dimethylglycine | 1.2 | 3.1 | Lipid and amino acid metabolism |
| 16 | Ile | 1.6 | 2.9 | BCAA & aromatic amino acids |
| 17 | Phe | 1.5 | 2.9 | BCAA & aromatic amino acids |
| 18 | Propionic acid | 1.3 | 2.9 | Lipid and amino acid metabolism/BCAA & aromatic amino acids |
| 19 | 2-Aminoisobutyric acid | 1.2 | 2.9 | BCAA & aromatic amino acids/Nucleotide metabolism |
| 20 | Cadaverine | 1.2 | 2.9 | Poly amine, Lysine degradation |
| 21 | Lactic acid | 1.1 | 2.9 | Central carbon metabolism/Urea cycle relating metaboloism |
| 22 | 2,3-Dihydroxy-isovalerate | 1.4 | 2.9 | Organic Acids |
| 23 | N-Acetylputrescine | 1.5 | 2.8 | Urea cycle relating metaboloism |
| 24 | 3-(4-Hydroxyphenyl)propionic acid | 1.1 | 2.8 | Lipid and amino acid metabolism/BCAA & aromatic amino acids |
| 25 | Isopropanolamine | 0.9 | 2.8 | Polyols |
| 26 | Indole-3-acetic acid | 1.2 | 2.7 | Plant hormone-of the auxin class |
| 27 | Pro | 0.9 | 2.7 | Urea cycle relating metaboloism |
| 28 | 5-Valerolactam | 1.2 | 2.6 | 5-AVA degradation |
| 29 | Citronellol glucoside | 0.9 | 2.6 | Terpenes |
| 30 | 3-Indolebutyric acid | 1.3 | 2.5 | Plant hormone in the auxin family |
| 31 | Putroscine | 1.3 | 2.5 | Urea cycle relating metaboloism |
| 32 | 1H-Imidazole-4-propionic acid; | 1.9 | 2.4 | salivary stimutant |
| 33 | γ-Butyrobetaine | 1.7 | 2.4 | Lipid and amino acid metabolism |
| 34 | 3-Phenylpropionic acid | 1.2 | 2.4 | Phenyl acids |
| 35 | Valeric acid | 1.1 | 2.4 | BCAA & aromatic amino acids |
| 36 | 3-(4-Methyl-3-pentenyl)thiophene | 1.5 | 2.3 | Terpenos |
| 37 | Sarcosine | 1.5 | 2.3 | Lipid and amino acid metabolism |
| 38 | Methylbenzoic acid | 1.2 | 2.3 | Phenyl acids |
| 39 | Phosphoric acid | 1.2 | 2.3 | Organic Acids |
| 40 | Toluic acid | 1.3 | 2.2 | Organic Acids |
| 41 | Glycerol 3-phosphate | 0.0 | 2.2 | Central carbon metabolism/Lipid and amino acid metabolism |
| 42 | Dopamine | 1.2 | 2.1 | Neurotransmitters |
| 43 | XC0001 | 2.1 | 2.1 | Unknown |
| 44 | Tyr | 1.5 | 2.1 | BCAA & aromatic amino acids |
| 45 | p-Hydroxyphenylacetic acid | 1.1 | 2.1 | Phenyl acids |
| 46 | Hexanoic acid | 1.0 | 2.1 | Organic Acids |
| 47 | 5-Oxoproline | 3.5 | 2.0 | Urea cycle relating metaboloism |
| 48 | Taurine | 1.4 | 2.0 | Lipid and amino acid metabolism |
| 49 | O-Acetylhomoserine | 1.3 | 2.0 | Cysteine and methionine metabolism |
| 50 | N-Acetylneuraminic acid (NANA) | 1.4 | 2.0 | Amino sugar and nucleotide sugar metabolism |
| 51 | 3-Aminobutyric acid | 1.6 | 1.9 | Neurotransmitters |
| 52 | Trimethylamine | 1.3 | 1.9 | Methane metabolism |
| 53 | Gly | 1.0 | 1.9 | Urea cycle relating metaboloism/Lipid and amino acid metabolism |
| 54 | Uric acid | 1.0 | 1.9 | Nucleotide metabolism |
| 55 | Trp | 1.6 | 1.8 | BCAA & aromatic amino acids |
| 56 | Creatine | 1.2 | 1.8 | Urea cycle relating metaboloism |
| 57 | Phosphorylcholine | 1.1 | 1.8 | Lipid and amino acid metaboloism |
| 58 | Homovanillic acid | 1.4 | 1.7 | BCAA & aromatic amino acids |
| 59 | Crotonic acid | 1.2 | 1.7 | Organic compounds |
| 60 | Isocitric acid | 0.0 | 1.7 | Central carbon metabolism |
| 60 | Isocitric acid | 0.0 | 1.7 | Central carbon metabolism |
| 61 | Ala | 1.3 | 1.6 | Central carbon metabolism/Urea cycle relating metaboloism/BCAA |
| | | | | & aromatic amino acids |
| 62 | β-Ala | 1.0 | 1.6 | Urea cycle relating metaboloism/Nucleotide metabolism/ |
| | | | | Metabolism of coenzymes |
| 63 | N-Acetylaspartic acid | 0.0 | 1.6 | Urea cycle relating metaboloism |
| 64 | 3-Phenyllactic acid | 1.3 | 1.5 | Phenyl acids |
| 65 | XA0033 | 1.1 | 1.5 | Unknown |
| 64 | Urea | 0.8 | 1.5 | Urea cycle relating metaboloism |
| 67 | O-Acetylcarnitine | 0.7 | 1.5 | Lipid and amino acid metabolism |
| 68 | Glu | 1.8 | 1.4 | Central carbon metabolism/Urea cycle relating metaboloism |
| 69 | Gln | 1.5 | 1.4 | Urea cycle relating metaboloism |
| 70 | Citric acid | 0.6 | 1.4 | Central carbon metabolism |
| 71 | His | 1.7 | 1.3 | Urea cycle relating metaboloism |
| 72 | Spermidine | 2.0 | 1.2 | Urea cycle relating metaboloism |
| 73 | Diethanolamine | 1.9 | 1.2 | Glycerophospholipid metabolism |
| 74 | Creatinine | 1.2 | 1.2 | Urea cycle relating metaboloism |
| 75 | Urocanic acid | 6.4 | 1.1 | Urea cycle relating metaboloism |
| 76 | Asp | 2.0 | 1.1 | Central carbon metabolism/Urea cycle relating metaboloism/ |
| | | | | Nucleotide metabolism |
| 77 | O-Acetylserina | 2.0 | 1.1 | Cysteine and methionine metabolism |
| 78 | Thr | 2.1 | 1.0 | Lipid and amino acid metabolism |
| 79 | Ser | 1.7 | 0.9 | Lipid and amino acid metabolism |
| 80 | Adenine | 2.1 | 0.8 | Nucleotide metabolism |
| 81 | Nicotinic acid | 3.2 | 0.0 | Metabolism of coenzymes |
| 82 | Hypoxanthine | 2.2 | 0.0 | Nucleotide metabolism |
| 83 | Met | 1.8 | 0.0 | Lipid and amino acid metabolism |
| 84 | Pyruvic acid | 1.8 | 0.0 | Central carbon metabolism/Urea cycle relating metaboloism/Lipid |
| | | | | and amino acid metabolism |
| 85 | 1,3-Diaminopropane | 1.6 | 0.0 | Urea cycle relating metaboloism |

metabolite designated as “>10”. Pathway Index describes the categories on the basis of metabolic pathway (KEGG) and biological functions (HMDB) of candidate compounds.

Table 6 contains a list of 20 short peptides (2-4 amino acids in length) that show differences post-consumption (verses pre-consumption) in subject 3 and Table 7 contains a list of 19 short proline peptides (2-7 amino acids in length) that show differences pre- and post-consumption in subject 3.

Human saliva contains about 2,400 different proteins of which 200-300 are of gland secretion origin that belong to the following major families: α-amylases, carbonic anhydrase, histatins, mucins, proline-rich proteins (PRPs), statherin, P-B peptide, and salivary-type (S-type) cystatins (Ekstrom et al., 2017). PRPs functions include lubrication, mineralization, tissue coating, binding of tannins and antiviral lubrication.

The changes in saliva flow-rate and content were demonstrated by Kopach et al. (2012). Saliva from ducts of the submandibular gland in rats treated with the cannabinoids binding receptor (CBR) agonist WIN55212-2T, and the specific CB1

TABLE 6
Basic peptides that significantly change post-consumption in subject 3

| | | | | Fold of |
|---|---|---|---|---|
| | | Fold of | Fold of | change |
| | | change | change | after |
| # | Peptides | after 10 min | after 60 min | 120 min |
| 1 | Arg Phe Ile Trp | <10 | <34 | <15 |
| 2 | Phe Ser | 0.7 | 4.8 | 1.3 |
| 3 | 5-Amino-1- | 1.2 | 4.0 | 1.4 |
| | ribofuranosylimidazole- | | | |
| | 4-carboxyamide | | | |
| 4 | His Thr Ile Ala | 1.2 | 3.9 | 0.6 |
| 5 | Lys Ser His Thr | 1.0 | 3.2 | 1.3 |
| 6 | Lys Ser Asp Gly | 0.9 | 2.7 | 0.0 |
| 7 | Phe Phe Thr Ile | 1.1 | 2.6 | 0.9 |
| 8 | Lys Phe Gln | 1.5 | 2.5 | 2.1 |
| 9 | Trp Ser Leu Phe | 1.6 | 2.4 | 1.1 |
| 10 | Lys Ser Asp Gly | 1.0 | 2.4 | 1.4 |
| 11 | Arg His Ala | 1.4 | 2.4 | 1.0 |
| 12 | His Ile Ile Met | 1.5 | 2.4 | 1.4 |
| 13 | Arg Tyr Asn Met | 1.6 | 2.4 | 1.5 |
| 14 | His Ala Cys | 0.3 | 2.3 | 0.8 |
| 15 | Trp Ser Leu Asp | 1.0 | 2.2 | 1.4 |
| 16 | Arg Gln Gln | 1.2 | 2.1 | 0.7 |
| 17 | Lys Ser Thr | 1.3 | 2.1 | 0.9 |
| 18 | Arg Phe Tyr Asp | 1.2 | 2.1 | 1.0 |
| 19 | Arg Thr Glu Arg | 0.9 | 1.8 | 1.1 |
| 20 | Arg Tyr Tyr Val | 0.9 | 1.8 | 1.3 |

receptor agonist (R)-(+)-methanandamide and the CB2 receptor agonist JWH015 caused a significant decrease in saliva flow rate accompanied by an increase in total protein content and Ca 2+ concentration with no changes in other electrolytes. Similarly, saliva collected from subject 3 exhibits increases in basic peptide and proline peptides, see Tables 6 and 7 respectively.

A search of the literature for examples of small peptides as biomarkers is documented for: 1) β-amyloid 1-42 and tau protein in the cerebrospinal fluid for Alzheimer's disease; 2) Combinations of peptides in the urine for diabetic nephropathy, chronic kidney disease, acute kidney injury, acute renal allograft rejection, prostate cancer, and coronary artery disease (Dallas et al. 2015).

TABLE 7
Proline peptides that significantly change post-consumption in subject 3
Patient 3 Pro Peptides

| | | Fold of | Fold of | Fold of |
|---|---|---|---|---|
| | | change after | change after | change after |
| # | Peptides | 10 min | after 60 min | 120 min |
| 1 | Pro Pro Gly | 2.6 | 7.3 | 2.6 |
| 2 | Pro Pro Lys Gly | 1.2 | 4.9 | 1.7 |
| 3 | Pro Pro | 1.2 | 4.8 | 2.2 |
| 4 | HydroxyPro Val | 0.8 | 4.0 | 2.0 |
| 5 | Pro Pro Pro | 1.1 | 4.0 | 2.0 |
| 6 | Pro Lys | 1.4 | 3.8 | 1.7 |
| 7 | Pro Ser Asn Leu | 1.4 | 3.4 | 1.7 |
| 8 | Pro Asn Thr | 1.2 | 3.1 | 1.4 |
| 9 | Pro Lys Gly | 1.2 | 2.8 | 1.9 |
| 10 | Pro His Trp Ser | 1.8 | 2.6 | 1.8 |
| 11 | Pro Arg Gln Gln Asn | 1.5 | 2.6 | 2.0 |
| 12 | Pro Arg Gln Gln | 1.3 | 2.6 | 1.0 |
| 13 | Pro Gly Asn Gln | 1.0 | 2.5 | 1.1 |
| 14 | Pro Tyr Lys Gly | 1.0 | 2.1 | 1.6 |
| 15 | Pro Trp Trp Leu | 0.9 | 2.0 | 1.1 |
| 16 | Trp Trp Leu, Pro Arg | 0.7 | 1.9 | 1.2 |
| | Thr Met | | | |
| 17 | Pro Trp Leu Gln | 1.1 | 1.9 | 1.5 |
| 18 | Pro Phe Gly Arg | 1.0 | 1.9 | 1.6 |
| 19 | Pro Asn Arg Ser | 0.9 | 1.6 | 1.2 |

Table 8 contains a list of the biochemical pathways that show differences post-consumption (verses pre-consumption) in subject 3. The number of biomarker candidates per biochemical pathway is indicated.

TABLE 8
Number of biomarker candidates showing differences post-consumption
per biochemical pathway in subject 3

| | Number |
|---|---|
| Pathway Index | Candidates |
| Polyamines, amino acids, | 20 |
| Basic peptides | 20 |
| Proline peptide | 19 |
| Organic acids | 9 |
| Neurotransmitters, Stimulants | 5 |
| Osmolytes | 5 |
| Phenyl acids | 4 |
| Plant hormones | 4 |
| Phenyl acids | 4 |
| Polyamines | 4 |
| Branched-chain amino acids (BCAAs) | 3 |
| TCA cycle | 3 |
| BCAA | 3 |
| Polyols | 2 |
| Terpennes | 2 |
| Biofilms | 2 |
| Osmolytes | 2 |
| Saliva stimulants | 2 |
| Steroids | 2 |
| Thiols | 2 |
| Unknown | 2 |
| Glucose Metabolism | 1 |
| Peptidase activity | 1 |
| Depression Biomarker | 1 |
| Energy metabolism | 1 |
| Choline | 1 |
| FA transport | 1 |
| Total | 125 |

Table 9 describes 56 known and unknown metabolites detected in subject 4 10 min and 60 min post-consumption. Pathway Index describes the categories on the basis of metabolic pathway (KEGG) and biological functions (HMDB) of candidate compounds.

TABLE 9
Compounds that significantly change post-consumption in subject 4

| | | Fold of change | Fold of change | |
|---|---|---|---|---|
| # | Compound | after 10 min | after 60 min | Pathway Index |
| 1 | Spermine | 6.8 | 0.5 | Urea cycle relating metabolism |
| 2 | Trimethylamine | 5.7 | 0.0 | Methane metabolism |
| 3 | 3-Phenylpropionic acid | 4.2 | 0.0 | Phenyl acids |
| 4 | XA0033 | 3.0 | 1.2 | UnKnown |
| 5 | Phosphorylcholine | 3.0 | 1.0 | Lipid and amino acid metabolism |
| 6 | Glycerol 3-phosphate | 2.4 | 0.9 | Central carbon metabolism/Lipid and amino acid metabolism |
| 7 | Lys | 2.3 | 1.2 | Lipid and amino acid metabolism |
| 8 | Sarcosine | 2.3 | 1.2 | Lipid and amino acid metabolism |
| 9 | Cadaverine | 2.3 | 1.1 | Polyamine, Lysine degradation |
| 10 | Hydroxyproline | 2.3 | 0.7 | Urea cycle relating metaboloism |
| 11 | γ-Butyrobetaine | 2.2 | 1.2 | Lipid and amino acid metabolism |
| 12 | Taurine | 2.1 | 0.8 | Lipid and amino acid metabolism |
| 13 | GABA | 2.0 | 1.6 | Urea cycle relating metaboloism |
| 14 | Gly | 2.0 | 1.4 | Urea cycle relating metaboloism/Lipid and amino acid metabolism |
| 15 | Hypoxanthine | 2.0 | 0.4 | Nucleotide metabolism |
| 16 | Ile | 1.9 | 2.1 | BCAA & aromatic amino acids |
| 17 | Butyric acid | 1.9 | 1.5 | Organic Acids |
| 18 | Valeric acid | 1.9 | 1.4 | BCAA & aromatic amino acids |
| 19 | O-Acetylcarnitine | 1.9 | 1.0 | Lipid and amino acid metabolism |
| 20 | 3-Hydroxybutyric acid | 1.9 | 0.8 | Central carbon metabolism/Lipid and amino acid metabolism |
| 21 | p-Hydroxyphenylacetic acid | 1.8 | 2.2 | Phenyl acids |
| 22 | Pro | 1.8 | 2.1 | Urea cycle relating metaboloism |
| 23 | Gln | 1.8 | 0.8 | Urea cycle relating metaboloism |
| 24 | Toluic acid | 1.7 | 1.8 | Xylene degradation |
| 25 | Putrescine | 1.7 | 1.6 | Urea cycle relating metaboloism |
| 26 | Dihydroxyacetone phosphate | 1.7 | 1.1 | Central carbon metabolism/Lipid and amino acid metabolism |
| 27 | Uric acid | 1.7 | 1.1 | Nucleotide metabolism |
| 28 | 3-Aminobutyric acid | 1.7 | 0.9 | Neurotransmitters |
| 29 | Choline | 1.7 | 0.6 | Lipid and amino acid metabolism |
| 30 | Ornithine | 1.6 | 2.1 | Urea cycle relating metaboloism |
| 31 | Propionic acid | 1.6 | 2.1 | Lipid and amino acid metabolism/BCAA & aromatic amino acids |
| 32 | 5-Aminovaleric acid | 1.6 | 1.3 | Bacterial Transformation |
| 33 | Glu-Glu | 1.6 | 1.2 | Central carbon metabolism/Urea cycle relating metaboloism |
| 34 | Stachydrine | 1.6 | 1.2 | Phytochemical compounds |
| 35 | Creatine | 1.6 | 1.0 | Urea cycle relating metaboloism |
| 36 | 2-Aminoadipic acid | 1.6 | 1.0 | Lipid and amino acid metabolism |
| 37 | N-Acetylgalactosamine | 1.5 | 1.7 | Galactose metabolis |
| 38 | Arg | 1.5 | 1.2 | Central carbon metabolism/Urea cycle relating metaboloism |
| 39 | β-Ala | 1.5 | 0.8 | Urea cycle relating metaboloism/Nucleotide metabolism/Metabolism of |
| | | | | coenzymes |
| 40 | Citrulline | 1.5 | 0.7 | Urea cycle relating metaboloism |
| 41 | Glutathione (GSSG)_divalent | 1.5 | 0.3 | Urea cycle relating metaboloism |
| 42 | 1-Methyl-4-imidazoleacetic acid | 1.4 | 3.5 | Urea cycle relating metaboloism |
| 43 | Leu | 1.4 | 2.4 | BCAA & aromatic amino acids |
| 44 | 3-(4-Hydroxyphenyl)propionic | 1.4 | 1.8 | Lipid and amino acid metabolism/BCAA & aromatic amino acids |
| | acid | | | |
| 45 | Succinic acid | 1.3 | 2.5 | Central carbon metabolism/Urea cycle relating metaboloism |
| 46 | Glu | 1.3 | 1.6 | Central carbon metabolism/Urea cycle relating metaboloism |
| 47 | Phe | 1.2 | 1.7 | BCAA & aromatic amino acids |
| 48 | N-Acetylneuraminic acid (NANA) | 1.1 | 2.2 | Central carbon metabolism |
| 49 | Putrescine | 1.1 | 2.1 | Urea cycle relating metaboloism |
| 50 | Tyr | 1.1 | 2.1 | BCAA & aromatic amino acids |
| 51 | N¹,N⁸-Diacetylspermidine | 1.0 | 2.2 | Organic compounds |
| 52 | Ser | 0.9 | 1.7 | Lipid and amino acid metabolism |
| 53 | 2-Hydroxyglutaric acid | 0.9 | 1.6 | Butanoate metabolism |
| 54 | His | 0.7 | 2.7 | Urea cycle relating metaboloism |
| 55 | 5-Oxoproline | 0.7 | 2.6 | Urea cycle relating metaboloism |
| 56 | Anserine_divalent | 0.1 | 1.6 | Urea cycle relating metaboloism |

Table 10 contains a list of the biochemical pathways that showed differences post-consumption (verses pre-consumption) in subject 4. The number of biomarker candidates per biochemical pathway is indicated.

Metabolomic analysis was applied to saliva samples from 4 subjects and 151 compounds had significant differences in levels (over 50%) post vs. pre cannabis consumption. The major metabolic pathways affected by cannabis consumption in all subjects were: 1) Urea cycle relating metabolism; 2) Lipid and amino acid metabolism; 3) Nucleotide metabolism; 4) Branched-chain amino acid (BCAA) & aromatic amino acids; and 5) Central carbon metabolism/Lipid and amino acid metabolism.

### Example 2: Identification of General Metabolic Biomarker Candidates Indicative of Cannabis Consumption

A total of 211 metabolites were analyzed in the following Venn diagram indicating 70 biomarker candidates identified in subject 2 (medium gray), 85 biomarkers candidates identified in subject 3 (white) and 56 biomarkers candidates identified in subject 4 (light gray) (FIG. 8). The overlap (dark gray, dashed line) represents general

TABLE 10
Number of biomarker candidates showing differences post-consumption
per biochemical pathway in subject 4

| | Number |
|---|---|
| Pathway Index | Candidates |
| Urea cycle relating metaboloism | 15 |
| Lipid and amino acid melabolism | 9 |
| BCAA & aromatic amino acids | 5 |
| Central carbon metabolism/Lipid and amino | 4 |
| acid metabolism | |
| Central carbon metabolism/Urea cycle relating | 4 |
| metaboloism | |
| Lipid and amino acid metabolism/BCAA & | 2 |
| aromatic amino acids | |
| Nucleotide metabolism | 2 |
| Phenyl acids | 2 |
| Bacterial Transformation | 1 |
| Butanoate metabolism | 1 |
| Galactose metabolis | 1 |
| Methane metabolism | 1 |
| Neurotransmitters | 1 |
| Organic Acids | 1 |
| Organic compounds | 1 |
| Phytochemical compounds | 1 |
| Polyamine, Lysine degradation | 1 |
| Unknown | 1 |
| Urea cycle relating metaboloism/Lipid and | 1 |
| amino acid metabolism | |
| Urea cycle relating metaboloism/Nucleotide | 1 |
| metabolism/Metabolism of coenzymes | |
| Xylene degradation | 1 |
| Total | 56 |

metabolic biomarker candidates indicative of cannabis consumption common to all candidates.

The biomarker candidate 5-Oxoproline exhibits 40-350% increase 10 min post-consumption of cannabis and was not specific to consumption method, gender, age or medical condition described earlier in Table 1. 5-Oxoproline levels known to increase following the metabolic acidosis High Anion Gap Metabolic Acidosis (HAGMA). One possible explanation of the changes is provided by Verma et al. (2012). HAGMA caused by 5-oxoprolinuria resulted from chronic intermittent paracetamol therapy, malnutrition and concomitant moderate renal/hepatic dysfunction that includes a long history of cannabis consumption.

The increase of Lactic acid detected in all subjects directly affects the energy balance via the metabolic pathway of the TCA cycle in which the levels of the biomarker candidates Citric acid and Argenine (Arg) were reduced in all subjects (FIG. 9). Kanakis Jr (1976) showed that intravenous A9-THC equivalent to the amount delivered by one cannabis cigarette significantly increases heart rate and decreases Phosphoenolpyruvate (PEP) without any change in systolic or diastolic arterial pressure. It is possible that the decrease in the levels of PEP disrupts the metabolic homeostasis of energy regulation in cardiac tissue, potentially decreasing the levels of the biomarker candidates Citric acid and (Arg).

### Example 3: Applications of Specific Metabolic Biomarker Candidates Associated with Cannabis Consumption

Table 11 lists biomarker candidates that are unique to different factors such as gender, age, medical conditions, consumption method and cannabis product (e.g. flower, edibles, etc.). These biomarker candidates may include, but are not limited to the following applications:

1) C26H38O3 (steroidal): Not detected pre-consumption and significantly increases 10 min post-consumption and further increases 7-fold over the 10 min levels to 60 min post-consumption in subject 3. It does not exhibit a known function and is designated as “Surrogate Biomarker Candidate”, a measurable biomarker of a specific treatment that may correlate with a real clinical endpoint but does not necessarily have a guaranteed relationship. C26H38O3 can potentially be used as a biomarker for product and/or medical condition.

2) Theobromine (Alkaloid): Not detected pre-consumption and significantly increases 10 min post-consumption and then disappears after 60 min post-consumption in subject 2. Theobromine is a plant-based bitter methylxanthine derivative found in cocoa, tea plant leaves, and kola nut that can potentially indicate the specific product/cannabis strain (flower) and dosage consumed by the subject. Theobromine is an “External Biomarker Candidate”

3) C6H12O6 (Polyol): Not detected pre-consumption, 10 min post consumption and then significantly increases 60 min post-consumption in subject 3. C6H12O6 is an organic compound containing multiple hydroxyl groups that serves as a “Surrogate Biomarker Candidate”. It can potentially used as a biomarker for product and/or medical condition.

4) 2-Hydroxy-4-methylvaleric acid (Organic acid): Not detected pre-consumption, 10 min post consumption and then significantly increases 60 min post-consumption in subject 3. 2-Hydroxy-4-methylvaleric acid serves as a “Surrogate Biomarker Candidate”. It can potentially be used as a biomarker for product and/or medical condition.

5) 6-Aminooctahydroindolizin-1-yl acetate (indolizidine alkaloidal mycotoxin): Increases 1.3-fold 10 min post-consumption and 4.2-fold 60 min post-consumption in subject 3. It generally causes salivation and functions as a Slobber Factor. The increased levels of 6-Aminooctahydroindolizin-1-yl acetate is in accordance with the proline-peptides and the dry mouth phenomena caused by cannabis consumption. 6-Aminooctahydroindolizin-1-yl acetate can serve as a “Cannabis Consumption-dependent Biomarker”. The levels of 6-Aminooctahydroindolizin-1-yl acetate can potentially indicate time from last cannabis consumption, an important piece of information in drug use/abuse screening tests.

6) Ethanolamine phosphate (Glycerophospholipid): Decreases 0.4-fold 10 min post consumption and significantly increases 3.2-fold 60 min post-consumption in subject 3. Ethanolamine phosphate is a known biomarker in plasma for depressive state. Kawamura et al. (2018) demonstrated that phosphoethanolamine (PEA) was significantly lower in the Major Depressive Disorder (MDD) group than in the healthy control group. The increasing levels of PEA 60 min post consumption of cannabis in subject 3 (who consumes cannabis for anxiety treatment) is in accordance with the finding reported by Kawamura et al. (2018) indicating reduced anxiety levels upon increasing PEA levels. Similarly, taurine (organic compound) that increases 1.4-fold 10 min post consumption and 2.0-fold 60 min post-consumption in subject 3 is described by Kawamura et al. (2018) as an MMD biomarker. The levels of PEA and taurine can serve as “Anxiety biomarkers” and be of further use as “Efficacy Biomarker” to determine the best-fit product/strain and dosage for individualized anxiety treatment.

7) Citronellol glucoside (Terpene): Decreases 0.9-fold 10 min post consumption and increases 2.6-fold 60 min post-consumption in subject 3. Citronellol and other terpenoids such as a-terpineol found in cannabis are deeply sedating upon inhalation, even in low concentrations (Turner 1980). Citronellol can be conjugated to glucosides to form citronellol glucoside via phase II metabolism prior to excretion from cells in phase III. Citronellol glucoside is an “External Biomarker Candidate” that can potentially indicate the specific product/cannabis strain (flower) and dosage consumed by the subject.

8) Stachydrine (Phytochemical compounds): Increases 1.6-fold 10 min post consumption and 1.2-fold 60 min post-consumption in subject 4. Stachydrine is a major constituent of the Chinese Herb Feral cannabis, or Wild Marijuana, a wild-growing cannabis generally descended from industrial hemp plants. Stachydrine is an “External Biomarker Candidate” that can potentially indicate the specific product/cannabis strain (edible) and dosage consumed by the subject.

TABLE 11
Unique biomarker candidates showing differences post consumption

| | | Fold of change | Fold of change | | | |
|---|---|---|---|---|---|---|
| # | Compound | after 10 min | after 60 min | Pathway Index | Subject | Function |
| 1 | C26H38O3 (steroidal) | >10 | >70 | Glucose | Subject 3 | UnKnown |
| | | | | Metabolism | | |
| 2 | Theobromine | >10 | N.T | Alkaloid | Subject 2 | Bitter alkaloid found in plants |
| 3 | C6H12O6 | 0 | >10 | Polyols | Subject 3 | Organic compound containing |
| | | | | | | multiple hydroxyl groups |
| 4 | 2-Hydroxy-4-methylvaleric | 0 | >10 | Organic Acids | Subject 3 | UnKnown |
| | acid | | | | | |
| 5 | 6- | 1.3 | 4.2 | exocrine function, | Subject 3 | Slobber factor |
| | Aminooctahydroindolizin- | | | especially | | |
| | 1-yl acetate | | | salivation | | |
| 6 | Ethanolamine phosphate | 0.4 | 3.2 | Sphingolipids | Subject 3 | Biomarker in plasma for |
| | | | | metabolism | | depressive state |
| 7 | Taurine | 1.4 | 2.0 | Lipid and amino | Subject 3 | Biomarker in plasma for |
| | | | | acid metabolism | | depressive state |
| 8 | Citronellol glucoside | 0.9 | 2.6 | Terpenes | Subject 3 | Citronellol - metabolized |
| 9 | Stachydrine | 1.6 | 1.2 | Phytochemical | Subject 4 | Major Constituent of the Chinese |
| | | | | compounds | | Herb |

### Example 4

This example illustrates a processing overview of the cultivar recommendation engine; see FIG. 3. The cultivar recommendation example consists of the following steps:
(1) Biomarker detection and quantification: The biomarker (metabolite) data is identified from a saliva sample of known biomarkers (metabolites) relevant to the medical condition.
(2) The biomarkers are translated into a heat map based on quantity present.
(3) The Cannformatics platform algorithm identifies a match against the heat maps of successfully-treated consumers with similar pharmacokinetics/demographic characteristics (e.g., age, gender, race, BMI).
(4) The Cannformatics platform suggests strains based on similar heat map for consumers with similar pharmacokinetics/demographic characteristics (e.g., age, gender, race, BMI).

### Example 5

This example illustrates the processing overview of a dosage recommendation engine. See FIG. 4. The dosage recommendation example consists of the following steps:
(1) Biomarker detection and quantification: Data of known biomarkers (metabolites) relevant to the medical condition are acquired from saliva samples of a consumer and translated to numeric values (arbitrary units).
(2) Biomarker efficacy analysis: The biomarker is compared with the molecule reference value generated by the Cannformatics database based on consumers with a similar medical conditions and pharmacokinetics characteristics (e.g., age, gender, race) consuming a similar cultivar.
(3) Identification of cannabis molecules and concentrations for increasing efficacy: using artificial intelligence and machine learning technologies, the Cannformatics platform identifies the relevant molecules that will contribute to the delta-efficacy and translate the arbitrary units of each molecule to concentration (mg).
(4) Identification of a molecules vaporizing temperature and time: the Cannformatics platform determines the vaporizing temperature based on Table 13 and translates the concentration (mg) of each molecule contributing to the delta-efficacy to the Vaporizing time.
(5) Vaporizer program: The Cannformatics device adjusts the vaporizer to the time and temperature to match ideal, predicted efficacy.

TABLE 13
Cannabis constituents extraction. A process of thermal conversion of solid
organic matter to gas in a presence or absence of oxygen (combustion
or pyrolysis respectively) in which toxins gases commonly found in
cannabis smoke are formed.

| Method | Cannabinoids | Flavonoids | Terpenoids | BP° C. |
|---|---|---|---|---|
| Vaporizing | Δ9THCA | | | 104 |
| | CBDA | | | 120 |
| | | β-sitosterol | | 134 |
| | CBCA | | | 145 |
| | | α,β-pinene | | 155 |
| | | | α-terpinol | 156 |
| | Δ9THC | | | 157 |
| | | | β-myrcene | 167 |
| | | | Δ-3-carene | 168 |
| | | | 1,8-cineole | 176 |
| | | | D-limonene | 177 |
| | | | P-cymene | 177 |
| | | Apigenin | | 178 |
| | CBD | | | 180 |
| | | Cannflavin A | | 182 |
| | | | Linalool | 198 |
| | | | β- | 199 |
| | | | caryophyllene | |
| | | | Terpinol-4-ol | 209 |
| | Borneol | | | 210 |
| | | | α-terpineol | 217 |
| | CBC | | | 220 |
| | | | Pulegone | 224 |

| | | |
|---|---|---|
| Smoking | Combustion*/pyrolysis* | 232 |

### Example 6

This example illustrates a heat map of biomarkers of efficacy values obtained from cannabis-treated patients for pain for pre (Pr) and Post (Ps) consumption, plus values for healthy individuals with similar consumer factors (e.g., age, gender, ethnicity, Body Mass Index). See FIG. 5.

Table 12 lists the “known-unknown” peaks from CE-TOFMS measurement without annotation based on the chemical standards are shown in the label of “XA˜˜˜˜/XC˜˜˜˜”.

TABLE 12
Analytical Characteristics of “Known-Unknown” Peaks

| | | Candidate compounds | | | | | | |
|---|---|---|---|---|---|---|---|---|
| | | | | KEGG | | | | |
| ID | Mode¹ | Mass² | Formula³ | database | KEGG name | HMP database | HMP name | HMP Description |
| XC0001 | Cation | 71.073 | C4H9N | C12244 | 3-Buten-1-amine | | | |
| XC0016 | Cation | 128.058 | C5H8N2O2 | C00906 | 5,6-Dihydrothymine | HMDB0000079 | Dihydrothymine | Intermediate |
| | | | | | | | | breakdown |
| | | | | | | | | product of |
| | | | | | | | | thymine |
| | | | | C05717 | 2-Amino-4-cyanobutanoic | | | |
| | | | | | acid | | | |
| | | | | C05715 | 4-Amino-4-cyanobutanoic | | | |
| | | | | | acid | | | |
| XC0061 | Cation | 217.130 | C10H19NO4 | C03017 | O-Propanoylcarnitine | HMDB00824 | Propionylcarnitine | Associated |
| | | | | | | | | with propionic |
| | | | | | | | | acidemia |
| XA0013 | Anion | 173.999 | C6H6O4S | C00850 | Aryl sulfate | | | |
| | | | | C02180 | Phenylsulfate | | | |
| | | | | C12849 | p-Phenolsulfonic acid | | | |
| XA0019 | Anion | 192.027 | C6H8O7 | C00451 | D-threo-Isocitric acid | HMDB06511 | 2,3-Diketo-L- | An intermediate |
| | | | | | | | gulonate | in Ascorbate and |
| | | | | | | | | aldarate metabolism |
| | | | | C04617 | D-erythro-Isocitric acid | HMDB00094 | Citric acid | A weak acid that |
| | | | | | | | | is formed in the |
| | | | | | | | | tricarboxylic acid |
| | | | | | | | | cycle or that may be |
| | | | | | | | | introduced with diet |
| | | | | C04575 | 2,3-Diketo-L-gulonate | HMDB05971 | Diketogulonic acid | A metabolite of |
| | | | | | | | | the degradation |
| | | | | | | | | of vitamin C |
| | | | | C02780 | 2,5-Diketogluconic acid | HMDB00193 | Isocitric acid | The citrate oxidation |
| | | | | | | | | to isocitrate is |
| | | | | | | | | catalyzed by the |
| | | | | | | | | enzyme aconitase |
| | | | | C03921 | 2-Dehydro-3-deoxy-D- | HMDB01874 | D-threo-Isocitric | The substrate of |
| | | | | | glucarate | acid | | isocitrate |
| | | | | | | | | dehydrogenase |
| | | | | | | | | (IDH) |
| | | | | C00679 | 5-Dehydro-4-deoxy-D- | | | |
| | | | | | glucarate | | | |
| | | | | C03600 | Carboxymethyloxysuccinate | | | |
| | | | | C00158 | Citric acid | | | |
| | | | | C00311 | Isocitric acid | | | |
| XA0033 | Anion | 243.087 | C9H13N3O5 | C00475 | Cytidine | | | |
| | | | | C02961 | Cytaratine | | | |
| | | | | C05711 | gamma-Glutamyl-beta- | HMDB00089 | Cytidine | A nucleoside that |
| | | | | | cyanoalanine | | | is composed of |
| | | | | | | | | the base cytosine |

¹Molecular ions with positive and negative charge are measured in Cation and Anion Mode, respectively.
²Predicated mass value was calculated as mono-valent ion.
³Predicted Molecular formula was on the basis of measured m/z value.

### EXAMPLE EMBODIMENTS

### Embodiment 1

A method for producing a recommended treatment, the method comprising: receiving, by the one or more computing devices, a disease state database comprising: a disease state Metabolite profile indicating a disease state; a treatment regime for treating the disease state based on metabolite profile; receiving, by the one or more computing devices, a patient database comprising: a patient metabolite profile calculating, by the one or more computing devices, a correlation between the patient metabolite profile and the disease state metabolite profile; and generating, by the one or more computing devices, from the correlation between the patient metabolite profile and the disease state metabolite profile, a recommended treatment regime from the disease state database.

### Embodiment 2

The method for producing a recommended treatment of embodiment 1, wherein the disease state metabolite profile further comprises metabolites available in saliva.

### Embodiment 3

The method for producing a recommended treatment of embodiment 1, wherein the patient metabolite profile further comprises a pre-treatment patient metabolite profile.

### Embodiment 4

The method for producing a recommended treatment of embodiment 3, wherein the patient metabolite profile further comprises a post treatment patient metabolite profile.

### Embodiment 5

The method for producing a recommended treatment of embodiment 4, wherein the patient metabolite profile further comprises a plurality of post treatment patient metabolite profiles.

### Embodiment 6

The method for producing a recommended treatment of embodiment 1, further comprises: calculating a positive outcome score for the recommended treatment regime.

### Embodiment 7

The method for producing a recommended treatment of embodiment 1, wherein the calculating step comprises an artificial intelligence method, a machine learning method or a deep learning method.

### Embodiment 8

The method for producing a recommended treatment of embodiment 1, further comprising: obtaining a cultivar database comprising cultivar chemicals and metabolites.

### Embodiment 9

The method for producing a recommended treatment of embodiment 8, wherein the cultivar database further comprises a metabolite response by a population of patients in response to consuming a cultivar.

### Embodiment 10

The method for producing a recommended treatment of embodiment 8, wherein the calculating step further comprises calculating, by the one or more computing devices, a correlation between the patient metabolite profile, the cultivar database, and the disease state metabolite profile.

### Embodiment 11

The method for producing a recommended treatment of embodiment 8, further comprising: predicting an alternative treatment regime.

### Embodiment 12

The method for producing a recommended treatment of embodiment 11, further comprising: calculating a positive outcome score for the recommended treatment regime.

### Embodiment 13

A kit comprising a saliva sample collection device to measure at least one marker in a patient sample, wherein the at least one marker corresponds to at least one biomarker with a relationship to a component of a marijuana plant.

### Embodiment 14

The kit of embodiment 13, wherein the component of a marijuana plant may be selected from a group comprising: Alpha-2-pinene, Beta-2-pinene, myrcene, alpha-phellandrene, delta-3-carene, beta-phellandrene/R-limonene, cineol, cis-ocimene, gama-terpinene, terpinolene, (−)linalool, beta-fenchol, cis-sabinene hydrate, camphor, borneol, alpha-terpineol, cis-bergamotene, alpha-guaiene, aromadendrene, alpha-humulene, trans-beta-farnesene, gamma-selinene, delta-guaiene, gamma-cadinene, eudesma-3,7(11)-diene, gamma-elemene, nerolidol, trans-beta-caryophyllene, beta-caryophyllene oxide, guaiol, gamma-eudesmol, beta-eudesmol, alpha-bisabolol, THCV, CBD, CBC, CBGM, THC, and/or CBG.

### Embodiment 15

The kit of embodiment 13, wherein the marker in the patient sample may be selected from a group comprising: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg).

### Embodiment 16

A method for identifying a patient for treatment with a marijuana cultivar comprising: a) measuring at least one biomarker in a patient sample comprising saliva; b) identifying whether the at least one biomarker measured in step a) is informative for outcome upon treatment with a marijuana cultivar; and c) identifying the patient for treatment with the marijuana cultivar if step b) indicates that the saliva comprise at least one biomarker that indicates a favorable outcome to marijuana cultivar therapy, wherein the at least one biomarker is selected from a group comprising: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg).

### Embodiment 17

A method for treating a pain patient with a therapeutic regimen comprising: a) using a measurement of at least one biomarker of a patient, wherein at least one biomarker gene is selected from a group comprising of 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg), to select for treatment a patient who is expected to have a favorable outcome with the therapeutic regimen, and b) treating the pain patient with at least one component from a marijuana cultivar, wherein the at least one component from a marijuana cultivar is selected from a group comprising Alpha-2-pinene, Beta-2-pinene, myrcene, alpha-phellandrene, delta-3-carene, beta-phellandrene/R-limonene, cineol, cis-ocimene, gama-terpinene, terpinolene, (−)linalool, beta-fenchol, cis-sabinene hydrate, camphor, borneol, alpha-terpineol, cis-bergamotene, alpha-guaiene, aromadendrene, alpha-humulene, trans-beta-farnesene, gamma-selinene, delta-guaiene, gamma-cadinene, eudesma-3,7(11)-diene, gamma-elemene, nerolidol, trans-beta-caryophyllene, beta-caryophyllene oxide, guaiol, gamma-eudesmol, beta-eudesmol, alpha-bisabolol, THCV, CBD, CBC, CBGM, THC, and/or CBG.

### Embodiment 18

A method of treating pain in a subject, comprising administering a therapeutically effective amount of components from a marijuana cultivar to the subject, wherein the components from a marijuana cultivar are selected from a group comprising Alpha-2-pinene, Beta-2-pinene, myrcene, alpha-phellandrene, delta-3-carene, beta-phellandrene/R-limonene, cineol, cis-ocimene, gama-terpinene, terpinolene, (−)linalool, beta-fenchol, cis-sabinene hydrate, camphor, borneol, alpha-terpineol, cis-bergamotene, alpha-guaiene, aromadendrene, alpha-humulene, trans-beta-farnesene, gamma-selinene, delta-guaiene, gamma-cadinene, eudesma-3,7(11)-diene, gamma-elemene, nerolidol, trans-beta-caryophyllene, beta-caryophyllene oxide, guaiol, gamma-eudesmol, beta-eudesmol, alpha-bisabolol, THCV, CBD, CBC, CBGM, THC, and/or CBG.

### Embodiment 19

The method of embodiment 18, wherein the therapeutically effective amount of THC is 2% to 8% concentration.

### Embodiment 20

The method of embodiment 18, wherein the therapeutically effective amount of THC is 2.5% to 8% concentration.

### Embodiment 21

The method of embodiment 18, wherein the therapeutically effective amount of THC is 3% to 8% concentration.

### Embodiment 22

The method of embodiment 18, wherein the therapeutically effective amount of THC is 3.5% to 8% concentration.

### Embodiment 23

The method of embodiment 18, wherein the therapeutically effective amount of THC is 4% to 8% concentration.

### Embodiment 24

The method of embodiment 18, wherein the therapeutically effective amount of THC is 4.5% to 8% concentration.

### Embodiment 25

The method of embodiment 18, wherein the therapeutically effective amount of THC is 5% to 8% concentration.

### Embodiment 26

The method of embodiment 18, wherein the therapeutically effective amount of THC is 5.5% to 8% concentration.

### Embodiment 27

The method of embodiment 18, wherein the therapeutically effective amount of THC is 6% to 8% concentration.

### Embodiment 28

The method of embodiment 18, wherein the therapeutically effective amount of THC is 6.5% to 8% concentration.

### Embodiment 29

The method of embodiment 18, wherein the therapeutically effective amount of THC is 7% to 8% concentration.

### Embodiment 30

The method of embodiment 18, wherein the therapeutically effective amount of THC is 7.5% to 8% concentration.

### Embodiment 31

The method of embodiment 18, wherein the therapeutically effective amount of THC is 2% to 7.5% concentration.

### Embodiment 32

The method of embodiment 18, wherein the therapeutically effective amount of THC is 2.5% to 7.5% concentration.

### Embodiment 33

The method of embodiment 18, wherein the therapeutically effective amount of THC is 3% to 7.5% concentration.

### Embodiment 34

The method of embodiment 18, wherein the therapeutically effective amount of THC is 3.5% to 7.5% concentration.

### Embodiment 35

The method of embodiment 18, wherein the therapeutically effective amount of THC is 4% to 7.5% concentration.

### Embodiment 36

The method of embodiment 18, wherein the therapeutically effective amount of THC is 4.5% to 7.5% concentration.

### Embodiment 37

The method of embodiment 18, wherein the therapeutically effective amount of THC is 5% to 7.5% concentration.

### Embodiment 38

The method of embodiment 18, wherein the therapeutically effective amount of THC is 5.5% to 7.5% concentration.

### Embodiment 39

The method of embodiment 18, wherein the therapeutically effective amount of THC is 6% to 7.5% concentration.

### Embodiment 40

The method of embodiment 18, wherein the therapeutically effective amount of THC is 6.5% to 7.5% concentration.

### Embodiment 41

The method of embodiment 18, wherein the therapeutically effective amount of THC is 6.5% to 7.5% concentration.

### Embodiment 42

The method of embodiment 18, wherein the therapeutically effective amount of THC is 7% to 7.5% concentration.

### Embodiment 43

The method of embodiment 18, wherein the therapeutically effective amount of THC is 2% to 6.5% concentration.

### Embodiment 44

The method of embodiment 18, wherein the therapeutically effective amount of THC is 2.5% to 6.5% concentration.

### Embodiment 45

The method of embodiment 18, wherein the therapeutically effective amount of THC is 3% to 6.5% concentration.

### Embodiment 46

The method of embodiment 18, wherein the therapeutically effective amount of THC is 3.5% to 6.5% concentration.

### Embodiment 47

The method of embodiment 18, wherein the therapeutically effective amount of THC is 4% to 6.5% concentration.

### Embodiment 48

The method of embodiment 18, wherein the therapeutically effective amount of THC is 4.5% to 6.5% concentration.

### Embodiment 49

The method of embodiment 18, wherein the therapeutically effective amount of THC is 5% to 6.5% concentration.

### Embodiment 50

The method of embodiment 18, wherein the therapeutically effective amount of THC is 5.5% to 6.5% concentration.

### Embodiment 51

The method of embodiment 18, wherein the therapeutically effective amount of THC is 6% to 6.5% concentration.

### Embodiment 52

The method of embodiment 18, wherein the therapeutically effective amount of THC is 2% to 6% concentration.

### Embodiment 53

The method of embodiment 18, wherein the therapeutically effective amount of THC is 2.5% to 6% concentration.

### Embodiment 54

The method of embodiment 18, wherein the therapeutically effective amount of THC is 3% to 6% concentration.

### Embodiment 55

The method of embodiment 18, wherein the therapeutically effective amount of THC is 3.5% to 6% concentration.

### Embodiment 56

The method of embodiment 18, wherein the therapeutically effective amount of THC is 4% to 6% concentration.

### Embodiment 57

The method of embodiment 18, wherein the therapeutically effective amount of THC is 4.5% to 6% concentration.

### Embodiment 58

The method of embodiment 18, wherein the therapeutically effective amount of THC is 5% to 6% concentration.

### Embodiment 59

The method of embodiment 18, wherein the therapeutically effective amount of THC is 5.5% to 6% concentration.

### Embodiment 60

The method of embodiment 18, wherein the therapeutically effective amount of THC is 2% to 5.5% concentration.

### Embodiment 61

The method of embodiment 18, wherein the therapeutically effective amount of THC is 2.5% to 5.5% concentration.

### Embodiment 62

The method of embodiment 18, wherein the therapeutically effective amount of THC is 3% to 5.5% concentration.

### Embodiment 63

The method of embodiment 18, wherein the therapeutically effective amount of THC is 3.5% to 5.5% concentration.

### Embodiment 64

The method of embodiment 18, wherein the therapeutically effective amount of THC is 4% to 5.5% concentration.

### Embodiment 65

The method of embodiment 18, wherein the therapeutically effective amount of THC is 4.5% to 5.5% concentration.

### Embodiment 66

The method of embodiment 18, wherein the therapeutically effective amount of THC is 5% to 5.5% concentration.

### Embodiment 67

The method of embodiment 18, wherein the therapeutically effective amount of THC is 2% to 5% concentration.

### Embodiment 68

The method of embodiment 18, wherein the therapeutically effective amount of THC is 2.5% to 5% concentration.

### Embodiment 69

The method of embodiment 18, wherein the therapeutically effective amount of THC is 3% to 5% concentration.

### Embodiment 70

The method of embodiment 18, wherein the therapeutically effective amount of THC is 3.5% to 5% concentration.

### Embodiment 71

The method of embodiment 18, wherein the therapeutically effective amount of THC is 4% to 5% concentration.

### Embodiment 72

The method of embodiment 18, wherein the therapeutically effective amount of THC is 4.5% to 5% concentration.

### Embodiment 73

The method of embodiment 18, wherein the therapeutically effective amount of THC is 2% to 4.5% concentration.

### Embodiment 74

The method of embodiment 18, wherein the therapeutically effective amount of THC is 2.5% to 4.5% concentration.

### Embodiment 75

The method of embodiment 18, wherein the therapeutically effective amount of THC is 3% to 4.5% concentration.

### Embodiment 76

The method of embodiment 18, wherein the therapeutically effective amount of THC is 3.5% to 4.5% concentration.

### Embodiment 77

The method of embodiment 18, wherein the therapeutically effective amount of THC is 4% to 4.5% concentration.

### Embodiment 78

The method of embodiment 18, wherein the therapeutically effective amount of THC is 2% to 4% concentration.

### Embodiment 79

The method of embodiment 18, wherein the therapeutically effective amount of THC is 2.5% to 4% concentration.

### Embodiment 80

The method of embodiment 18, wherein the therapeutically effective amount of THC is 3% to 4% concentration.

### Embodiment 81

The method of embodiment 18, wherein the therapeutically effective amount of THC is 3.5% to 4% concentration.

### Embodiment 82

The method of embodiment 18, wherein the therapeutically effective amount of THC is 2% to 3.5% concentration.

### Embodiment 83

The method of embodiment 18, wherein the therapeutically effective amount of THC is 2.5% to 3.5% concentration.

### Embodiment 84

The method of embodiment 18, wherein the therapeutically effective amount of THC is 3% to 3.5% concentration.

### Embodiment 85

The method of embodiment 18, wherein the therapeutically effective amount of THC is 2% to 3% concentration.

### Embodiment 86

The method of embodiment 18, wherein the therapeutically effective amount of THC is 2.5% to 3% concentration.

### Embodiment 87

The method of embodiment 18, wherein the therapeutically effective amount of THC is 2% to 2.5% concentration.

### Embodiment 88

A method for identifying an agent suitable for the treatment and/or prophylaxis of pain, wherein said method comprises: (i) taking a first saliva sample from a patient and recording the biomarker levels in the first saliva sample; (ii) recording a patients pre-consumption subjective level of pain; (iii) the patient consuming a marijuana cultivar; (iv) taking a second saliva sample from the patient and recording a post consumption biomarker levels in the second saliva sample; (v) recording patients post consumption subjective level of pain; (vi) calculating a correlation between the biomarker levels in the first saliva sample, the post consumption biomarker levels in the second saliva sample, and a change between patients pre-consumption subjective level of pain and patients post consumption level of pain; and (vii) identifying said marijuana cultivar as an agent suitable for the treatment and/or prophylaxis of pain by identifying a positive correlation between consuming the marijuana cultivar, a reduction in patients subjective level of pain, and a change in biomarker levels recorded in the saliva sample.

### Embodiment 89

A method for predicting a best marijuana cultivar for therapeutic treatment of a patient, comprising: (A) measuring a first activation level of one or more biomarkers in a sample from the patient's saliva, wherein the one or more biomarkers are selected from the group consisting of: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg); and (B) comparing the activation level of (A) to positive and/or negative reference standards to determine if the biomarker is activated; wherein the activation level of (A) is determined by measuring the first level of the biomarker in the patient's saliva before consuming a marijuana cultivar and comparing the first level to a second level comprising measurements of the biomarker in the patient's saliva after consuming a marijuana cultivar; and wherein the activation of the one or more biomarkers indicates a therapeutic treatment for patient's disease.

### Embodiment 90

A method for predicting a best marijuana cultivar for therapeutic treatment of a pain patient, comprising: (A) measuring a first activation level of one or more biomarkers in a sample from the patient's saliva, wherein the one or more biomarkers are selected from the group consisting of: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg); and (B) comparing the activation level of (A) to positive and/or negative reference standards to determine if the biomarker is activated; wherein the activation level of (A) is determined by measuring the first level of the biomarker in the patient's saliva before consuming a marijuana cultivar and comparing the first level to a second level comprising measurements of the biomarker in the patient's saliva after consuming a marijuana cultivar; and wherein the activation of the one or more biomarkers indicates a therapeutic treatment for patient's pain.

### Embodiment 91

A method for predicting a best marijuana cultivar for therapeutic treatment of a patient with anxiety, comprising: (A) measuring a first activation level of one or more biomarkers in a sample from the patient's saliva, wherein the one or more biomarkers are selected from the group consisting of: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg); and (B) comparing the activation level of (A) to positive and/or negative reference standards to determine if the biomarker is activated; wherein the activation level of (A) is determined by measuring the first level of the biomarker in the patient's saliva before consuming a marijuana cultivar and comparing the first level to a second level comprising measurements of the biomarker in the patient's saliva after consuming a marijuana cultivar; and wherein the activation of the one or more biomarkers indicates a therapeutic treatment for patient's anxiety.

### Embodiment 92

A method for predicting a best marijuana cultivar for therapeutic treatment of a patient with depression, comprising: (A) measuring a first activation level of one or more biomarkers in a sample from the patient's saliva, wherein the one or more biomarkers are selected from the group consisting of: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg); and (B) comparing the activation level of (A) to positive and/or negative reference standards to determine if the biomarker is activated; wherein the activation level of (A) is determined by measuring the first level of the biomarker in the patient's saliva before consuming a marijuana cultivar and comparing the first level to a second level comprising measurements of the biomarker in the patient's saliva after consuming a marijuana cultivar; and wherein the activation of the one or more biomarkers indicates a therapeutic treatment for patient's depression.

### Embodiment 93

A method for predicting a best marijuana cultivar for therapeutic treatment of a patient with cancer, comprising: (A) measuring a first activation level of one or more biomarkers in a sample from the patient's saliva, wherein the one or more biomarkers are selected from the group consisting of: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg); and (B) comparing the activation level of (A) to positive and/or negative reference standards to determine if the biomarker is activated; wherein the activation level of (A) is determined by measuring the first level of the biomarker in the patient's saliva before consuming a marijuana cultivar and comparing the first level to a second level comprising measurements of the biomarker in the patient's saliva after consuming a marijuana cultivar; and wherein the activation of the one or more biomarkers indicates a therapeutic treatment for patient's cancer.

### Embodiment 94

A method for predicting a best marijuana cultivar for therapeutic treatment of a patient with glaucoma, comprising: (A) measuring a first activation level of one or more biomarkers in a sample from the patient's saliva, wherein the one or more biomarkers are selected from the group consisting of: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg); and (B) comparing the activation level of (A) to positive and/or negative reference standards to determine if the biomarker is activated; wherein the activation level of (A) is determined by measuring the first level of the biomarker in the patient's saliva before consuming a marijuana cultivar and comparing the first level to a second level comprising measurements of the biomarker in the patient's saliva after consuming a marijuana cultivar; and wherein the activation of the one or more biomarkers indicates a therapeutic treatment for patient's glaucoma.

### Embodiment 95

A method for modifying a patients biomarker levels, comprising: taking a first sample of patients saliva; making a first measurement of patients biomarker levels; obtaining a database of marijuana cultivars comprising effects of marijuana cultivars on saliva biomarkers; obtaining a desired biomarker level of one or more biomarkers; and calculating, based on the database of marijuana cultivars and the first measurement of patients biomarker levels, which marijuana cultivar will result in the desired biomarker levels.

### Embodiment 96

The method of embodiment 94, further comprising: identifying the marijuana cultivar with highest probability of resulting in the desired biomarker levels.

### Embodiment 97

The method of embodiment 95, further comprising: recommending to the patient the marijuana cultivar.

### Embodiment 98

The method of embodiment 96, further comprising: taking a second sample of patients saliva after the patient has consumed the recommended marijuana cultivar; making a second measurement of patients biomarker levels; comparing the difference between the first measurement of patients biomarker levels and the second measurement of patients biomarker levels to the desired biomarker level of one or more biomarkers; and calculating which marijuana cultivar from the database of marijuana cultivars will result in the desired biomarker levels.

### Embodiment 99

The method of embodiment 97, further comprising: identifying the marijuana cultivar with highest probability of resulting in the desired biomarker levels.

### Embodiment 100

The method of embodiment 98, further comprising: recommending to the patient the marijuana cultivar.

### Embodiment 101

A method for predicting a best marijuana cultivar for therapeutic treatment of a patient with alzheimer disease, comprising: (A) measuring a first activation level of one or more biomarkers in a sample from the patient's saliva, wherein the one or more biomarkers are selected from the group consisting of: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acidb, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg); and (B) comparing the activation level of (A) to positive and/or negative reference standards to determine if the biomarker is activated; wherein the activation level of (A) is determined by measuring the first level of the biomarker in the patient's saliva before consuming a marijuana cultivar and comparing the first level to a second level comprising measurements of the biomarker in the patient's saliva after consuming a marijuana cultivar; and wherein the activation of the one or more biomarkers indicates a therapeutic treatment for patient's alzheimer disease.

### Embodiment 102

A method for identifying biomarkers indicative of consumption or presence of microorganisms, the method comprising: receiving, by the one or more computing devices, a disease state database comprising: a disease state Metabolite profile indicating a disease state; receiving, by the one or more computing devices, a patient database comprising: a patient metabolite profile; calculating, by the one or more computing devices, a correlation between the patient metabolite profile and the disease state metabolite profile; and generating, by the one or more computing devices, from the correlation between the patient metabolite profile and the disease state metabolite profile, and identification of a disease state.

### Embodiment 103

The method of embodiment 102, wherein the disease state metabolite profile further comprises metabolites and chemicals available in saliva.

### Embodiment 104

The method of embodiment 102, wherein the patient metabolite profile further comprises a pre-consumption patient metabolite profile.

### Embodiment 105

The method of embodiment 104, wherein the patient metabolite profile further comprises a post-consumption patient metabolite profile.

### Embodiment 106

The method of embodiment 105, wherein the patient metabolite profile further comprises a plurality of post-consumption patient metabolite profiles.

### Embodiment 107

The method of embodiment 102, wherein the calculating step comprises an artificial intelligence method, a machine learning method, a quantum computing method or a deep learning method.

### Embodiment 108

The method of embodiment 102, further comprising: obtaining a cultivar database comprising cultivar chemicals and metabolites.

### Embodiment 109

The method embodiment 108, wherein the cultivar database further comprises a metabolite response by a population of patients in response to consuming a cultivar.

### Embodiment 110

The method of embodiment 108, wherein the cultivar database comprises cultivars treated with pesticides.

### Embodiment 111

The method of embodiment 108, wherein the cultivar database comprises cultivars contaminated with microorganisms from a group consisting essentially of Shiga toxin-producing Escherichia coli, Salmonella spp, Aspergillus fumigatus, Aspergillus flavus, Aspergillus niger, Aspergillus terreus, Botrytis (mold) and/or Powdery Mildew.

### Embodiment 112

The method of embodiment 108, wherein the cultivar database comprises cultivars treated with pesticides selected a group consisting essentially of Abamectin, Acephate, Acequinocyl, Acetamiprid, Aldicarb, Azoxystrobin, Bifenazate, Bifenthrin, Boscalid, Captan, Carbaryl, Carbofuran, Chlorantraniliprole, Chlordane, Chlorfenapyr, Chlorpyrifos, Clofentezine, Coumaphos, Cyfluthrin, Cypermethrin, Daminozide, DDVP (Dichlorvos), Diazinon, Dimethoate, Dimethomorph, Ethoprop(hos), Etofenprox, Etoxazole, Fenhexamid, Fenoxycarb, Fenpyroximate, Fipronil, Flonicamid, Fludioxonil, Hexythiazox, Imazalil, Imidacloprid, Kresoxim-methyl, Malathion, Metalaxyl, Methiocarb, Methomyl, Methyl parathion, Mevinphos, Myclobutanil, Naled, Oxamyl, Paclobutrazol, Pentachloronitrobenzene, Permethrin, Phosmet, Piperonyl butoxide, Prallethrin, Propiconazole, Propoxur, Pyrethrins, Pyridaben, Spinetoram, Spinosad, Spiromesifen, Spirotetramat, Spiroxamine, Tebuconazole, Thiacloprid, Thiamethoxam and/or Trifloxystrobi.

### Embodiment 113

The method of embodiment 108, wherein the calculating step further comprises calculating, by the one or more computing devices, a correlation between the patient metabolite profile, the cultivar database, and the disease state metabolite profile.

### REFERENCES

Barnes V M, Kennedy A D, Panagakos F, Devizio W, Trivedi H M, Jonsson T, Guo L, Cervi S, Scannapieco F A. Global metabolomic analysis of human saliva and plasma from healthy and diabetic subjects, with and without periodontal disease. PLoS One. 2014 Aug. 18; 9(8):e105181. doi: 10.1371/journal.pone.0105181. eCollection 2014. Erratum in: PLoS One. 2014; 9(11):e114091. PubMed PMID: 25133529; PubMed Central PMCID: PMC4136819.
Commisso M, Strazzer P, Toffali K, Stocchero M, Guzzo F. Untargeted metabolomics: an emerging approach to determine the composition of herbal products. Comput Struct Biotechnol J 2013; 4 ( ): e201301007. PMID: 24688688 DOI: 10.5936/csbj.201301007
Cuevas-Córdoba B, Santiago-García J. Saliva: a fluid of study for OMICS. OMICS. 2014 February; 18(2):87-97. doi: 10.1089/omi.2013.0064. Epub 2014 Jan. 3. Review. PubMed PMID: 24404837.
Fiehn O. Metabolomics—the link between genotypes and phenotypes. Plant Mol Biol 2002; 48 (1-2): 155-71. PMID: 11860207
Jacobs R, Tshehla E, Malherbe S, Kriel M, Loxton A G, Stanley K, van der Spuy G, Walzl G, Chegou N N. Host biomarkers detected in saliva show promise as markers for the diagnosis of pulmonary tuberculosis disease and monitoring of the response to tuberculosis treatment. Cytokine. 2016 May; 81:50-6. doi: 10.1016/j.cyto.2016.02.004. Epub 2016 Feb. 13. PubMed PMID: 26878648.
Gutiérrez A M, Nöbauer K, Soler L, Razzazi-Fazeli E, Gemeiner M, Cerón J J, Miller I. Detection of potential markers for systemic disease in saliva of pigs by proteomics: a pilot study. Vet Immunol Immunopathol. 2013 Jan. 15; 151(1-2):73-82. doi: 10.1016/j.vetimm.2012.10.007. Epub 2012 Nov. 2. PubMed PMID: 23177629.
Karlik M, Valkovič P, Hančinová V, Križová L, Tóthová L′, Celec P. Markers of oxidative stress in plasma and saliva in patients with multiple sclerosis. Clin Biochem. 2015 January; 48(1-2):24-8. doi: 10.1016/j.clinbiochem.2014.09.023. Epub 2014 Oct. 7. PubMed PMID: 25304914.
Liu J, Duan Y. Saliva: a potential media for disease diagnostics and monitoring. Oral Oncol. 2012 July; 48(7):569-77. doi: 10.1016/j.oraloncology.2012.01.021. Epub 2012 Feb. 19. Review. PubMed PMID: 22349278.
Malottki K, Biswas M, Deeks J J, Riley R D, Craddock C, Johnson P, Billingham L. Stratified medicine in European Medicines Agency licensing: a systematic review of predictive biomarkers. BMJ Open. 2014 Jan. 27; 4(1):e004188. doi: 10.1136/bmjopen-2013-004188. Review. PubMed PMID: 24468721; PubMed Central PMCID: PMC3913033.
Zhang A, Sun H, Wang X. Saliva metabolomics opens door to biomarker discovery, disease diagnosis, and treatment. Appl Biochem Biotechnol. 2012 November; 168(6):1718-27. doi: 10.1007/s12010-012-9891-5. Epub 2012 Sep. 13. Review. PubMed PMID: 22971835.
Dallas, David C., et al. “Current peptidomics: applications, purification, identification, quantification, and functional analysis.” Proteomics 15.5-6 (2015): 1026-1038.
Ekström, Jorgen, et al. “Saliva and the control of its secretion.” (2017): 1-37.
Kopach, Olga, et al. “Cannabinoid receptors in submandibular acinar cells: functional coupling between saliva fluid and electrolytes secretion and Ca2+ signalling.” J Cell Sci (2012): jcs-088930.
Verma, Rajanshu, et al. “5-Oxoprolinuria as a cause of high anion gap metabolic acidosis.” British journal of clinical pharmacology 73.3 (2012): 489-491.
Kanakis Jr, Charles, Jean Maurice Pouget, and Kenneth M. Rosen. “The effects of delta-9-tetrahydrocannabinol (cannabis) on cardiac performance with and without beta blockade.” Circulation 53.4 (1976): 703-707.
Kawamura, Noriyuki, et al. “Plasma metabolome analysis of patients with major depressive disorder.” Psychiatry and clinical neurosciences 72.5 (2018): 349-361.
Turner, Carlton E., Mahmoud A. Elsohly, and Edward G. Boeren. “Constituents of Cannabis sativa L. XVII. A review of the natural constituents.” Journal of Natural Products 43.2 (1980): 169-234.

What is claimed is:

1. A method for producing a recommended treatment, the method comprising:
receiving, by the one or more computing devices, a disease state database comprising:
a disease state Metabolite profile indicating a disease state;
a treatment regime for treating the disease state based on metabolite profile;
receiving, by the one or more computing devices, a patient database comprising:
a patient metabolite profile;
calculating, by the one or more computing devices, a correlation between the patient metabolite profile and the disease state metabolite profile; and
generating, by the one or more computing devices, from the correlation between the patient metabolite profile and the disease state metabolite profile, a recommended treatment regime from the disease state database.

2. The method for producing a recommended treatment of claim 1, wherein the disease state metabolite profile further comprises metabolites available in saliva.

3. The method for producing a recommended treatment of claim 1, wherein the patient metabolite profile further comprises a pre-treatment patient metabolite profile.

4. The method for producing a recommended treatment of claim 3, wherein the patient metabolite profile further comprises a post treatment patient metabolite profile.

5. The method for producing a recommended treatment of claim 4, wherein the patient metabolite profile further comprises a plurality of post treatment patient metabolite profiles.

6. The method for producing a recommended treatment of claim 1, further comprises:
calculating a positive outcome score for the recommended treatment regime.

7. The method for producing a recommended treatment of claim 1, wherein the calculating step comprises an artificial intelligence method, a machine learning method or a deep learning method.

8. The method for producing a recommended treatment of claim 1, further comprising:
obtaining a cultivar database comprising cultivar chemicals and metabolites.

9. The method for producing a recommended treatment of claim 8, wherein the cultivar database further comprises a metabolite response by a population of patients in response to consuming a cultivar.

10. The method for producing a recommended treatment of claim 8, wherein the calculating step further comprises calculating, by the one or more computing devices, a correlation between the patient metabolite profile, the cultivar database, and the disease state metabolite profile.

11. The method for producing a recommended treatment of claim 8, further comprising:
predicting an alternative treatment regime.

12. The method for producing a recommended treatment of claim 11, further comprising:
calculating a positive outcome score for the recommended treatment regime.

13. A kit comprising a saliva sample collection device to measure at least one marker in a patient sample, wherein the at least one marker corresponds to at least one biomarker with a relationship to a component of a marijuana plant.

14. The kit of claim 13, wherein the component of a marijuana plant may be selected from a group comprising: Alpha-2-pinene, Beta-2-pinene, myrcene, alpha-phellandrene, delta-3-carene, beta-phellandrene/R-limonene, cineol, cis-ocimene, gama-terpinene, terpinolene, (−)linalool, beta-fenchol, cis-sabinene hydrate, camphor, borneol, alpha-terpineol, cis-bergamotene, alpha-guaiene, aromadendrene, alpha-humulene, trans-beta-farnesene, gamma-selinene, delta-guaiene, gamma-cadinene, eudesma-3,7(11)-diene, gamma-elemene, nerolidol, trans-beta-caryophyllene, beta-caryophyllene oxide, guaiol, gamma-eudesmol, beta-eudesmol, alpha-bisabolol, THCV, CBD, CBC, CBGM, THC, and/or CBG.

15. The kit of claim 13, wherein the marker in the patient sample may be selected from a group comprising: 2-Methylsuccinic acid, 3-Methylhistidine, 4-Hydroxyphenyllactate, 5-Aminopentanoic acid, Acetic acid, Acetoacetic acid, Acetone, Acetylcholine, Acetylglycine, Acetylornithine, Alpha-Hydroxyisobutyric acid, Alpha-Hydroxyisovaleric acid, Betaine, Butyric acid, Caffeine, Carnosine, Choline, Citric acid, Creatine, Creatinine, Cresol sulfate, D-Galactose, D-Glucose, Dimethyl sulfone, Dimethylamine, Dimethylarginine, Dimethylglycine, Ethanol, Ethanolamine, Formic acid, Fumaric acid, Galactitol, Gluconic acid, Glyceric acid, Glycerol, Glycerophosphocholine, Glycine, Glycolic acid, Histamine, Hydrocinnamic acid, Hydroxyisocaproic acid, Hydroxyproline, Hypoxanthine, Indole-3-acetic acid, Isocaproic acid, Isopropyl alcohol, Isovaleric acid, L-Alanine, L-Arginine, L-Aspartic acid, L-Citrulline, L-Fucose, L-Glutamic acid, L-Glutamine, L-Histidine, L-Isoleucine, L-Lactic acid, L-Leucine, L-Lysine, L-Malic acid, L-Methionine, L-Ornithine, L-Phenylalanine, L-Proline, L-Serine, L-Threonine, L-Tryptophan, L-Tyrosine, L-Valine, Malic acid, Methanol, Methionine sulfoxide, Methylamine, Methylguanidine, Methylsuccinic acid, Myo-inositol, Nicotinic acid, Palmitic acid, Phenylacetic acid, Phenylacetylglycine, Phenyllactic acid, Phosphoric acid, Phosphorylcholine, P-Hydroxybenzoic acid, P-Hydroxyphenylacetic acid, Propionic acid, Propylene glycol, Putrescine, Pyroglutamic acid, Pyruvic acid, Sarcosine, Sorbitol, Spermidine, Spermine, Stearic acid, Succinic acid, Taurine, Trimethylamine, Uracil, Urea, Valeric acid, Acylcarnitines (L-Carnitine, Tetradecanoyl-L-carnitine, Hexadecanoyl-L-carnitine, Hexadecadienyl-L-carnitine, Acetyl-L-carnitine, Propionyl-L-carnitine, Propenoyl-L-carnitine, Butyryl-L-carnitine, Valeryl-L-carnitine, Hydroxyhexanoyl-L-carnitine, Methylglutaryl-L-carnitine), Sphingomyelins (SM (OH) C16:1, SM (OH) C22:1, SM (OH) C22:2, SM (OH) C24:1, SM C16:0, SM C16:1, SM C18:0, SM C18:1, SM C24:0, SM C24:1, SM C26:1, H1/Glucose), Lysophosphatidylcholines (lysoPC a C14:0, lysoPC a C18:0, lysoPC a C20:4), Phosphatidylcholines (PC aa C32:0, PC aa C34:1, PC aa C34:2, PC aa C34:3, PC aa C36:1, PC aa C36:2, PC aa C36:3, PC aa C36:4, PC aa C36:5, PC aa C38:3, PC aa C38:4, PC aa C38:5, PC aa C38:6, PC aa C42:1, PC aa C42:2, PC aa C42:4, PC ae C32:1, PC ae C34:0, PC ae C34:1, PC ae C34:2, PC ae C36:1, PC ae C36:2, PC ae C36:3, PC ae C36:4, PC ae C38:0, PC ae C38:3, PC ae C38:4, PC ae C38:5, PC ae C40:2, PC ae C40:5, PC ae C42:2, PC ae C42:3, PC ae C44:3, PC ae C44:4, PC ae C44:5), Metal ions (Aluminium (Al), Arsenic (As), Barium (Ba), Boron (B), Calcium (Ca), Cerium (Ce), Cesium (Cs), Chromium (Cr), Cobalt (Co), Copper (Cu), Gallium (Ga), Germanium (Ge), Hafnium (Hf), Iron (Fe), Lanthanum (La), Lead (Pb), Lithium (Li), Magnesium (Mg), Manganese (Mn), Molybdenum (Mo), Neodymium (Nd), Nickel (Ni), Niobium (Nb), Palladium (Pd), Phosphorus (P), Platinum (Pt), Potassium (K), Rubidium (Rb), Selenium (Se), Sodium (Na), Strontium (Sr), Tellurium (Te), Thallium (TI), Tin (Sn), Titanium (Ti), Tungsten (W), Vanadium (V), Yttrium (Y), Zinc (Zn), Zirconium (Zr)), Vitamins (Vitamin A, Vitamin K3, Vitamin B9, Vitamin B6-Phosphate, Vitamin B5, Vitamin C, Vitamin B3, Vitamin B2), Nucleotides (Adenosine, Adenosine monophosphate, Adenosine triphosphate, Dihydrouracil, Inosine, Thymine, Uridine 50-monophosphate, Uridine triphosphate, Xanthine), and Thiols (Cysteine, Cysteinylglycine, Glutathione, Homocysteine, Glutamyl-Cysteineg).

16. A method of treating pain in a subject, comprising administering a therapeutically effective amount of components from a marijuana cultivar to the subject, wherein the components from a marijuana cultivar are selected from a group comprising Alpha-2-pinene, Beta-2-pinene, myrcene, alpha-phellandrene, delta-3-carene, beta-phellandrene/R-limonene, cineol, cis-ocimene, gama-terpinene, terpinolene, (−)linalool, beta-fenchol, cis-sabinene hydrate, camphor, borneol, alpha-terpineol, cis-bergamotene, alpha-guaiene, aromadendrene, alpha-humulene, trans-beta-farnesene, gamma-selinene, delta-guaiene, gamma-cadinene, eudesma-3,7(11)-diene, gamma-elemene, nerolidol, trans-beta-caryophyllene, beta-caryophyllene oxide, guaiol, gamma-eudesmol, beta-eudesmol, alpha-bisabolol, THCV, CBD, CBC, CBGM, THC, and/or CBG.

17. The method of claim 16, wherein a therapeutically effective amount of THC is 2% to 8% concentration.

18. The method of claim 16, wherein a therapeutically effective amount of THC is 2.5% to 8% concentration.

19. The method of claim 16, wherein a therapeutically effective amount of THC is 3% to 8% concentration.

20. The method of claim 16, wherein a therapeutically effective amount of THC is 3.5% to 8% concentration.
